(19) United States
(12) Patent Application Publication
(10) Pub. No.: US 2016/0223559 A1
(43) Pub. Date: Aug. 4, 2016

(54) TETRAZINE-CONTAINING COMPOUNDS AND SYNTHETIC METHODS THEREOF

(71) Applicant: The Regents of the University of California, Oakland, CA (US)

(72) Inventors: Neal K. Devaraj, Oakland, CA (US); Haoxing Wu, Oakland, CA (US); Jun Yang, Oakland, CA (US)

(21) Appl. No.: 15/013,921

(22) Filed: Feb. 2, 2016

Related U.S. Application Data
(60) Provisional application No. 62/110,760, filed on Feb. 2, 2015.

(51) Int. Cl. G01N 33/58; C07D 409/04; C07D 409/14; C07D 491/06; B01J 31/22; C07H 19/06; C07D 405/10; C07F 5/02; B01J 31/24; C07D 257/08; C07D 403/06

(52) U.S. Cl. CPC G01N 33/582; C07D 257/08; C07D 409/04; C07D 409/14; C07D 491/06; C07D 403/06; C07H 19/06; C07D 405/10; C07F 5/022; B01J 31/2409; B01J 31/22; B01J 31/24; B01J 2531/824; B01J 2231/40

(57) ABSTRACT

Described herein are tetrazine derivatives and efficient synthetic methods of synthesis thereof using elimination-Heck cascade reaction. Provided herein is the synthesis of conjugated tetrazines from the tetrazine derivatives. Also provided herein are methods of use of the conjugated tetrazines as fluorogenic probes for live-cell imaging.

### CROSS-REFERENCES TO RELATED APPLICATIONS

This application claims the benefit of U.S. Provisional Application No. 62/110,760, filed Feb. 2, 2015, which is incorporated herein by reference in its entirety and for all purposes.

### STATEMENT AS TO RIGHTS TO INVENTIONS MADE UNDER FEDERALLY SPONSORED RESEARCH AND DEVELOPMENT

This invention was made with government support under grant number K01EB010078 awarded by the National Institutes of Health. The Government has certain rights in the invention.

### BACKGROUND OF THE INVENTION

The chemistry of tetrazine-containing compounds such as 1,2,4,5-tetrazines has gained growing interest in the last decade, owing to their unique physicochemical characteristics.[1] Tetrazines have seen expanding use in chemical biology, material science, natural product synthesis, coordination chemistry, electrochemistry, photovoltaics, and explosives research. [1a,1b,2] Of particular interest has been the use of tetrazine containing compounds for bioorthogonal live-cell imaging applications.[1b,1c,3] In spite of the application potential of tetrazines, a major limitation has been the lack of practical synthetic methods. This has hampered the development of new fluorescent tetrazine probes, particularly those with fluorogenic properties.

Recently a metal-catalyzed one-pot procedure to prepare symmetric and unsymmetric tetrazines from aliphatic nitriles and anhydrous hydrazine was performed.[7] The technique has limitations. Synthesis requires excess anhydrous hydrazine and heating, conditions that are not compatible with several functional groups such as carbonyls and alkyl halides, that are susceptible to either nucleophilic addition or reduction.[8] It is therefore difficult to directly introduce 1,2,4,5-tetrazine onto relatively complex molecules such as fluorophores using this method. Conjugated alkenyl substituted 1,2,4,5-tetrazines were not obtainable from the corresponding alkenyl-nitriles. Additionally, there is limited commercial availability of anhydrous hydrazine in Europe and China due to safety concerns, further encumbering methods that require anhydrous hydrazine every time a new tetrazine derivative is synthesized.

Therefore, there is a need for practical chemical synthesis routes for tetrazine containing compounds. Provided herein are solutions to these and other problems in the art.

### BRIEF SUMMARY OF THE INVENTION

In a first aspect, there is provided a method of synthesizing a compound of Formula I:

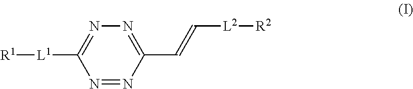

the method including reacting a compound of Formula II:

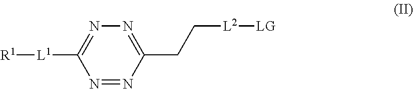

with a compound of Formula III:

R²—X (III),

wherein the compounds of Formula II and III are reacted in the presence of a Pd catalyst and a ligand under basic conditions. In the compounds of Formulas II and III, L¹ and L² are independently a bond or a covalent linker. R¹ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a biomolecule, a dye or fluorophore. R² is substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl or a biomolecule, a dye or fluorophore. In the compound of Formula II, LG is a leaving group. In the compound of Formula III, X is halogen.

In another aspect, there is provided a compound of Formula I:

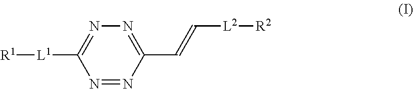

In the compound of Formula I, L¹ and L² are independently a bond or linker. R¹ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a biomolecule, a dye or fluorophore. R² is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl or a biomolecule, a dye or fluorophore.

In another aspect, there is provided a compound of Formula IV:

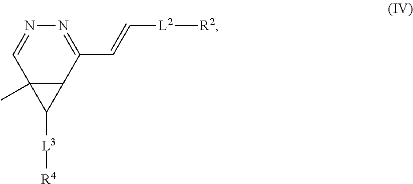

In the compound of Formula IV, L² and L³ are independently a bond or linker. R² is substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl or a biomolecule, a dye or fluorophore. R⁴ is a biomolecule, a dye or fluorophore.

In another aspect, there is provided a method of detecting a biomolecule of interest. The method includes:

(i) contacting the compound of Formula I as disclosed herein, wherein R² is a fluorophore, with a compound of Formula V:

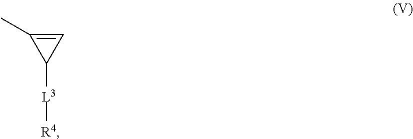

and
(ii) detecting the level of fluorescence, wherein an increase in fluorescence compared to a control is indicative of the presence of the biomolecule. In the compound of Formula V, L³ is a covalent bond or linker. R⁴ is the biomolecule.

### BRIEF DESCRIPTION OF THE DRAWINGS

FIGS 1A-1D. Depicted in FIG. 1A are fluorescence emission spectra for compound 2v (lowest line) and compound 5 (middle line) and 6 (upper line) in PBS; excitation at 480 nm. FIG. 1B: Figure depicts equimolar solutions of compound 6 and 2v under excitation by a handheld UV lamp. FIGS. 1C-1D: Figures depict Live-cell imaging of LS174T cells. FIG. 1C: Cells were incubated (t=1 h) with 200 nM TCO-conjugated A33 antibodies, washed, and then imaged 30 minutes after the addition of 5 μM 2v. FIG. 1D: Cells lacking TCO were treated with 5 μM 2v for 30 minutes. Scale bar=15 m for both FIGS. 1C and 1D.

FIGS. 2A-2C. FIG. 2A: Tetrazines 2a, 2b, and 2c (as indicated by the corresponding curves) at 1 mM were incubated at 22° C. Tetrazine decomposition in 1:1 MDF:PBS buffer, pH 7.4 was observed for 48-hrs. Tetrazine absorption peak intensities at around 520-530 nm were measured over time and plotted after baseline adjustments as a percent tetrazine remaining. FIG. 2B: Tetrazine stability observations in phosphate-buffer saline (PBS) after 24-h. Tetrazines 2c, 2a, and 2b at 1 mM were incubated in 50% DMF, 50% PBS 1×pH 7.4 at 22° C., and the fraction remaining was measured as a function of the disappearance of the characteristic 520 nm absorption peak intensity over time. FIG. 2C: Percent tetrazine remaining after 3 h in the presence of 1 mM cysteine.

FIGS. 3A-3B. (E)-3-substituted-6-styryl-s-tetrazine reaction kinetics. FIG. 3A: Reaction scheme of (E)-cyclooct-4-enol and the series of tetrazines for kinetic characterization. Only one isomer is depicted for 15. FIG. 3B: Tetrazine kinetics were determined by reacting 1 mM tetrazine with an excess (10 mM) of (E)-cyclooct-4-enol in 50% DMF, 50% PBS 1×pH 7.4 buffer at 22° C. Second-order rate constants derived from the data are listed, and the corresponding curves for 2b, 2p, and 11 are shown in FIG. 4A. Tetrazine 2p was reacted in 80% DMF, 20% PBS due to its insolubility at lower DMF concentrations.

FIGS. 4A-4B. Reaction kinetics of 1 mM tetrazines 2b, 2p, and 11 with 10 mM (E)-cyclooct-4-enol in 1:1 DMF:PBS pH 7.4 buffer at 22° C. are depicted in FIG. 4A. Data were collected under pseudo first-order conditions (data points plotted) and fitted to a single exponential decay (curves indicated). Tetrazine compounds are labelled next to the corresponding data curves. FIG. 4B: Figure shows extended time points for the reaction of tetrazine 2b with (E)-cyclooct-4-enol.

FIGS. 5A-5E. Fluorescence emission of 2v, 5, 6 are depicted in FIG. 1A. All three compounds were dissolved in 2 μM phosphate-buffered saline (PBS) at pH 7.4. FIG. 5A: Fluorescence emission of 2v in 2 μM phosphate-buffered saline (PBS) at pH 7.4. FIG. 5B: Fluorescence emission of 2w, 7, 8. All three compounds were dissolved in 2 μM EtOH solution. Compound 2w and 8 were tested after HPLC purification and 7 was tested directly after reaction. FIG. 5C: Fluorescence emission of 2w in 2 μM EtOH solution. FIG. 5D: Fluorescence emission of 2x, 9, 10. All three compounds were dissolved in 2 μM EtOH solution. Compounds 2x and 10 were tested after HPLC purification and compound 9 was tested directly after reaction. FIG. 5E: Fluorescence emission of 2x in 2 μM EtOH solution.

### DETAILED DESCRIPTION OF THE INVENTION

### I. Definitions

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain, or combination thereof, which may be fully saturated, mono- or polyunsaturated and can include di- and multivalent radicals, having the number of carbon atoms designated (i.e., C₁-C₁₀ means one to ten carbons). Alkyl is not cyclized. Examples of saturated hydrocarbon radicals include, but are not limited to, groups such as methyl, ethyl, n-propyl, isopropyl, n-butyl, t-butyl, isobutyl, sec-butyl, (cyclohexyl)methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—).

The term “alkylene,” by itself or as part of another substituent, means, unless otherwise stated, a divalent radical derived from an alkyl, as exemplified, but not limited by, —CH₂CH₂CH₂CH₂—. Typically, an alkyl (or alkylene) group will have from 1 to 24 carbon atoms. A “lower alkyl” or “lower alkylene” is a shorter chain alkyl or alkylene group, generally having eight or fewer carbon atoms.

The term “heteroalkyl,” by itself or in combination with another term, means, unless otherwise stated, a stable straight or branched chain, or combinations thereof, consisting of at least one carbon atom and at least one heteroatom selected from the group consisting of O, N, P, S, Se and Si, and wherein the nitrogen, selenium, and sulfur atoms may optionally be oxidized, and the nitrogen heteroatom may optionally be quaternized. Heteroalkyl is not cyclized. The heteroatom(s) 0, N, P, S, Se, and Si may be placed at any interior position of the heteroalkyl group or at the position at which the alkyl group is attached to the remainder of the molecule. Examples include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, —O—CH₃, —O—CH₂—CH₃, and —CN. Up to two heteroatoms may be consecutive, such as, for example, —CH₂—NH—OCH₃.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SeR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl,” by themselves or in combination with other terms, mean, unless otherwise stated, cyclic versions of“alkyl” and “heteroalkyl,” respectively. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (e.g. 1 to 3 rings) that are fused together (i.e., a fused ring aryl) or linked covalently. A fused ring aryl refers to multiple rings fused together wherein at least one of the fused rings is an aryl ring. The term “heteroaryl” refers to aryl groups (or rings) that contain from one to four heteroatoms (e.g. N, O, or S), wherein sulfur heteroatoms are optionally oxidized, and the nitrogen heteroatoms are optionally quaternized. Thus, the term “heteroaryl” includes fused ring heteroaryl groups (i.e., multiple rings fused together wherein at least one of the fused rings is a heteroaromatic ring). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively.

A fused ring heterocyloalkyl-aryl is an aryl fused to a heterocycloalkyl. A fused ring heterocycloalkyl-heteroaryl is a heteroaryl fused to a heterocycloalkyl. A fused ring heterocycloalkyl-cycloalkyl is a heterocycloalkyl fused to a cycloalkyl. A fused ring heterocycloalkyl-heterocycloalkyl is a heterocycloalkyl fused to another heterocycloalkyl. Fused ring heterocycloalkyl-aryl, fused ring heterocycloalkyl-heteroaryl, fused ring heterocycloalkyl-cycloalkyl, or fused ring heterocycloalkyl-heterocycloalkyl may each independently be unsubstituted or substituted with one or more of the substituents described herein. Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g. substituents for cycloalkyl or heterocycloalkyl rings). Spirocylic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g. all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The term “oxo,” as used herein, means an oxygen that is double bonded to a carbon atom.

The term “alkylsulfonyl,” as used herein, means a moiety having the formula —S(O₂)—R′, where R′ is an alkyl group as defined above. R′ may have a specified number of carbons (e.g., “C₁-C₄ alkylsulfonyl”).

The term “oxy” as used herein, alone or in combination, refers to —O—.

The term “aryloxy” as used herein, alone or in combination, refers to a substituted or unsubstituted aryl group attached to the parent molecular moiety through an oxy i.e. an ether group. An example of an unsubstituted aryl ether group is phenoxy (i.e. C₆H₅O—).

The term “heteroaryloxy” as used herein, alone or in combination, refers to a substituted or unsubstituted heteroaryl group attached to the parent molecular moiety through an oxy i.e. a heteroaryl ether group. An example of an unsubstituted heteroaryl ether group is thiophenyl (i.e. C₄H₃SO—).

Each of the above terms (e.g., “alkyl,” “heteroalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, ═N—OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, and —NO₂ in a number ranging from zero to (2m′+1), where m′ is the total number of carbon atoms in such radical. R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy, or thioalkoxy groups, or arylalkyl groups. When a compound of the invention includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-,6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, -halogen, —SiR′R″R′″, —OC(O)R′, —C(O)R′, —CO₂R′, —CONR′R″, —OC(O)NR′R″, —NR″C(O)R′, —NR′—C(O)NR″R′″, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN, —NO₂, —R′, —N₃, —CH(Ph)₂, fluoro(C₁-C₄)alkoxy, and fluoro(C₁-C₄)alkyl, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound of the invention includes more than one R group, for example, each of the R groups is independently selected as are each R′, R″, R′″, and R″″ groups when more than one of these groups is present.

Substituents for rings (e.g. cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g. a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

Two of the substituents on adjacent atoms of the aryl or heteroaryl ring may optionally form a ring of the formula -T-C(O)—(CRR′)_{q}—U—, wherein T and U are independently —NR—, —O—, —CRR′—, or a single bond, and q is an integer of from 0 to 3. Alternatively, two of the substituents on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′—, or a single bond, and r is an integer of from 1 to 4. One of the single bonds of the new ring so formed may optionally be replaced with a double bond. Alternatively, two of the substituents on adjacent atoms of the aryl or heteroaryl ring may optionally be replaced with a substituent of the formula —(CRR′)ₛ—X′— (C″R′″)_{d}—, where s and d are independently integers of from 0 to 3, and X′ is —O—, —NR′—, —S—, —S(O)—, —S(O)₂—, or —S(O)₂NR′—. The substituents R, R′, R″, and R′″ are preferably independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) —OH, —NH₂, —SH, —CN, —CF₃, —NO₂, oxo, halogen, unsubstituted alkyl, unsubstituted heteroalkyl, unsubstituted cycloalkyl, unsubstituted heterocycloalkyl, unsubstituted aryl, unsubstituted heteroaryl, and
(B) alkyl, heteroalkyl, cycloalkyl, heterocycloalkyl, aryl, and heteroaryl, substituted with at least one substituent selected from:
   (i) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
   (ii) alkyl, heteroalkyl, cycloalkyl, heterocycloalkyl, aryl, and heteroaryl, substituted with at least one substituent selected from:
      (a) oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, unsubstituted heteroaryl, and
      (b) alkyl, heteroalkyl, cycloalkyl, heterocycloalkyl, aryl, or heteroaryl, substituted with at least one substituent selected from: oxo, —OH, —NH₂, —SH, —CN, —CF₃, —NO₂, halogen, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₅ cycloalkyl, and each substituted or unsubstituted heterocycloalkyl is a substituted or unsubstituted 3 to 8 membered heterocycloalkyl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, and each substituted or unsubstituted heterocycloalkyl is a substituted or unsubstituted 3 to 7 membered heterocycloalkyl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, and/or each substituted or unsubstituted heterocycloalkyl is a substituted or unsubstituted 3 to 8 membered heterocycloalkyl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₅ cycloalkylene, and/or each substituted or unsubstituted heterocycloalkylene is a substituted or unsubstituted 3 to 8 membered heterocycloalkylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, and/or each substituted or unsubstituted heterocycloalkyl is a substituted or unsubstituted 3 to 7 membered heterocycloalkyl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, and/or each substituted or unsubstituted heterocycloalkylene is a substituted or unsubstituted 3 to 7 membered heterocycloalkylene.

Certain compounds of the present invention possess asymmetric carbon atoms (optical or chiral centers) or double bonds; the enantiomers, racemates, diastereomers, tautomers, geometric isomers, stereoisometric forms that may be defined, in terms of absolute stereochemistry, as (R)- or (S)- or, as (D)- or (L)- for amino acids, and individual isomers are encompassed within the scope of the present invention. The compounds of the present invention do not include those which are known in art to be too unstable to synthesize and/or isolate. The present invention is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

The term “about” in the context of a numerical value means, unless indicated otherwise, the nominal numerical value+10% thereof.

It will be apparent to one skilled in the art that certain compounds of this invention may exist in tautomeric forms, all such tautomeric forms of the compounds being within the scope of the invention.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the invention.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this invention.

The compounds of the present invention may also contain unnatural proportions of atomic isotopes at one or more of the atoms that constitute such compounds. For example, the compounds may be radiolabeled with radioactive isotopes, such as for example tritium (³H), iodine-125 (¹²⁵I), or carbon-14 (¹⁴C). All isotopic variations of the compounds of the present invention, whether radioactive or not, are encompassed within the scope of the present invention.

The symbol “ [symbol] ” denotes the point of attachment of a chemical moiety to the remainder of a molecule or chemical formula.

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹³ substituents are present, each R¹³ substituent may be distinguished for example as R^{13A}, R^{13B}, R^{13C}, R^{13D}, etc., wherein each of R^{13A}, R^{13B}, R^{13C}, R^{13D}, etc. is defined within the scope of the definition of R¹³ and optionally differently.

Description of compounds of the present invention is limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

As used herein, the term “salt” refers to acid or base salts of the compounds used in the methods of the present invention. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts.

The term “tetrazine” refers in the customary sense to a six-membered ring containing four nitrogen atoms. Absent express indication otherwise, the term tetrazine as used herein refers to the isomer of tetrazine with formula 1,2,4,5-tetrazine. The term “symmetric” in the context of substitution of a chemical moiety, e.g., substitution of tetrazine, refers in the customary sense to disubstitution with the same substituent, e.g., 3,6-dimethyl-1,2,4,5-tetrazine. Conversely, the term “asymmetric” in this context refers to disubstitution with different substituents.

A “nitrile” refers to a organic compound having a —CN group.

A “protected secondary amine” refers to the covalent attachment of a monovalent chemical moiety to an amine nitrogen atom that functions to prevent the amine moiety from reacting with reagents used in the chemical synthetic methods described herein (commonly referred to as “protecting” the amine group) and may be removed under conditions that do not substantially degrade the molecule of which the amine moiety forms a part (commonly referred to as “deprotecting” the amine group) thereby yielding a free amine. An amine protecting group can be acid labile, base labile, or labile in the presence of other reagents. Amine protecting groups include but are not limited to: -carbamates (such as -carbobenzyloxy (Cbz), -t-butoxycarbonyl (t-Boc), -fluorenylmethyloxycarbonyl (Fmoc), and -allyl carbamates), -benzyl, -4-methoxyphenyl, or -2,4-dimethoxyphenyl.

“Contacting” is used in accordance with its plain ordinary meaning and refers to the process of allowing at least two distinct species (e.g. chemical compounds including biomolecules or cells) to become sufficiently proximal to react, interact or physically touch. It should be appreciated; however, the resulting reaction product can be produced directly from a reaction between the added reagents or from an intermediate from one or more of the added reagents which can be produced in the reaction mixture.

The terms “contacting” and “reacting” are used synonymously and may include allowing two species to react, interact, or physically touch, wherein the two species may be a compound as described herein and a protein or enzyme. In some embodiments contacting includes allowing a compound described herein to interact with a protein or enzyme that is involved in a signaling pathway.

The term “cascade reaction” as used herein refers to a chemical process that comprises at least two consecutive chemical reactions such that each subsequent reaction occurs only by virtue of the chemical functionality formed in the previous step. It should be appreciated that isolation of intermediates is not required, the reaction conditions do not change among the consecutive steps of a cascade and no new reagents are added after the initial step.

The term “one pot procedure” refers to a chemical procedure that allows at least two reactions to be carried out consecutively without isolation of intermediates. The addition of new reagents or the change of conditions after the first reaction, however, is not precluded.

The term “basic conditions” refers to conditions in which at least one base will be present. The bases contemplated herein include organic and/or inorganic bases. Basic conditions as used herein are intended to include organic solvents and inorganic solvents, including but not limited to aqueous solvent systems or any combination thereof.

The term “microwave-assisted” as used herein refers to reactions that are performed using radiation in the radio wave and/or microwave region of the electromagnetic spectrum, i.e., in the range of wavelengths of from about 1 mm to about 100 km. In embodiments, the radiation is in the microwave region.

“Microwave-assisted” reactions may be performed in an apparatus that generates microwaves where the reaction components are irradiated with microwaves. The microwave-assisted reactions as described herein may include microwave irradiation at a single wavelength or multiple wavelengths. The microwave-assisted reactions and procedures as described herein may be performed at a range of temperatures, for example at a temperature of from about 20° C. to about 200° C. In certain embodiments, the microwave-assisted reactions are performed at a temperature of from about 40° C. to 80° C.

As used herein, “biomolecule” is used in its customary sense and refers to a molecule that is present in living organisms and synthetic derivatives thereof, including macromolecules such as proteins, carbohydrates, lipids, and nucleic acids, as well as small molecules such as primary metabolites, secondary metabolites, and natural products. A biomolecule includes but is not limited to nucleic acids (e.g. DNA and RNA), peptide nucleic acids, sugars, peptides, proteins, antibodies, lipids, small molecule affinity ligands e.g. inhibitors, biotin and haptens.

The terms “phosphino” and “phosphine” as used herein refer to a compound having the formula PR′R″R′″ or (PR′R″R′″R″″)⁺•X⁻. The R groups in PR′R″R′″ and (PR′R″R′″R″″)⁺ are as described herein and may include, for example, hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl or substituted or unsubstituted heteroaryl. X⁻ in (PR′R″R′″R″″)⁺•X⁻ may be any suitable counter ion known in the art, for example, a borate anion. “Phosphine” and “phosphino” are used interchangeably herein.

“Phosphinoferrocene” as used herein refers to a phosphine-ferrocene or phosphinoferrocene complex typically with a phosphine moiety covalently bound to one or both of the cyclopentadienyl rings of the ferrocene compound. It should be appreciated that ferrocene refers to an organometallic compound with the formula Fe(C₅H₅)₂ comprising two cyclopentadienyl rings bound on opposite sides of a central iron atom.

“Ligand” as used herein refers to an ion or molecule that becomes attached to palladium by coordinate bonding. The term “ligand” may be alternatively referred to as “palladium ligand.” In certain embodiments, ligands are phosphine or phosphino groups and/or phosphinoferrocene complexes.

“Leaving group” or “LG” as used herein refers to an atom (or a group of atoms) that is displaced as a stable species taking with it the bonding electrons. The leaving group leaves as an anion (e.g. Cl⁻) or a neutral molecule (e.g. H₂O) for example, however, under certain reaction conditions, the leaving group leaves as a cation.

“Electron withdrawing group” as used herein refers to an atom or chemical group that draws electron density from neighboring atoms towards itself, usually by resonance (through electron delocalization where electrons are not fixed on specific atoms or bonds, but are spread out over several atoms or bonds) or inductive effects (electronic effect due to the polarization of a bonds within a molecule or ion). Examples of functional groups that are electron withdrawing groups include but are not limited to a nitrile group, a nitro group, ketones, aldehydes, esters, halides, perfluoroalkyl groups, aryloxy groups, heteroaryloxy groups, oxonium groups or quaternary amino groups.

The term “fluorophore” as used herein refers to a fluorescent chemical compound or moiety that can re-emit light upon light excitation. Fluorophores typically contain several combined aromatic groups, or planar or cyclic molecules with several it bonds.

The term “co-catalyst” as used herein refers to either of a pair of cooperative catalysts that improve each other's catalytic activity. In some embodiments, the co-catalysts are both Pd-metal catalysts. In other embodiments, the co-catalysts are not Pd-metal catalysts and may include, for example, Cu catalysts.

The term “Pd catalyst” as used herein refers to homogeneous or heterogeneous catalysts that contain Pd and are suitable for cross-coupling reactions. As used herein, “Pd catalyst” includes Pd salts alone and/or with the addition of suitable ligands.

The term “dienophile” as used herein refers to an alkene or alkyne that reacts with a conjugated diene in a cycloaddition reaction. As used herein, dienophiles may contain heteroatoms e.g. nitrogen. In some embodiments, the dienophiles are strained ring systems.

“Dye” is used in accordance with its plain ordinary meaning and refers to compounds or moieties that absorb light in the visible spectrum (400-700 nm), have at least one chromophore (color-bearing group), have a conjugated system of alternating double and single bonds and exhibit resonance of electrons e.g. xanthenes.

The term “linker” as described herein is a divalent chemical group that covalently joins one chemical moiety to another. Specific examples of linkers are described herein. Linkers may be polyethylene (PEG) linkers or bioconjugate linkers.

As used herein, the term “bioconjugate” or “bioconjugate linker” refers to the resulting association between atoms or molecules of bioconjugate reactive groups. The association can be direct or indirect. For example, a conjugate between a first bioconjugate reactive group (e.g., —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g. a first linker of second linker), or indirect, e.g., by non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). In embodiments, bioconjugates or bioconjugate linkers are formed using bioconjugate chemistry (i.e. the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D. C., 1982. In embodiments, the first bioconjugate reactive group (e.g., maleimide moiety) is covalently attached to the second bioconjugate reactive group (e.g. a sulfhydryl). In embodiments, the first bioconjugate reactive group (e.g., haloacetyl moiety) is covalently attached to the second bioconjugate reactive group (e.g. a sulfhydryl). In embodiments, the first bioconjugate reactive group (e.g., pyridyl moiety) is covalently attached to the second bioconjugate reactive group (e.g. a sulfhydryl). In embodiments, the first bioconjugate reactive group (e.g., —N-hydroxysuccinimide moiety) is covalently attached to the second bioconjugate reactive group (e.g. an amine). In embodiments, the first bioconjugate reactive group (e.g., maleimide moiety) is covalently attached to the second bioconjugate reactive group (e.g. a sulfhydryl). In embodiments, the first bioconjugate reactive group (e.g., -sulfo-N-hydroxysuccinimide moiety) is covalently attached to the second bioconjugate reactive group (e.g. an amine).

Useful bioconjugate reactive groups used for bioconjugate chemistries herein include, for example:
(a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters;
(b) hydroxyl groups which can be converted to esters, ethers, aldehydes, etc.
(c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom;
(d) dienophile groups which are capable of participating in Diels-Alder reactions such as, for example, maleimido or maleimide groups;
(e) aldehyde or ketone groups such that subsequent derivatization is possible via formation of carbonyl derivatives such as, for example, imines, hydrazones, semicarbazones or oximes, or via such mechanisms as Grignard addition or alkyllithium addition;
(f) sulfonyl halide groups for subsequent reaction with amines, for example, to form sulfonamides;
(g) thiol groups, which can be converted to disulfides, reacted with acyl halides, or bonded to metals such as gold, or react with maleimides;
(h) amine or sulfhydryl groups (e.g., present in cysteine), which can be, for example, acylated, alkylated or oxidized;
(i) alkenes, which can undergo, for example, cycloadditions, acylation, Michael addition, etc;
(j) epoxides, which can react with, for example, amines and hydroxyl compounds;
(k) phosphoramidites and other standard functional groups useful in nucleic acid synthesis;
(l) metal silicon oxide bonding; and
(m) metal bonding to reactive phosphorus groups (e.g. phosphines) to form, for example, phosphate diester bonds.
(n) azides coupled to alkynes using copper catalyzed cycloaddition click chemistry.
(o) biotin conjugate can react with avidin or strepavidin to form a avidin-biotin complex or streptavidin-biotin complex.

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate comprises a molecular entity derived from the reaction of an unsaturated bond, such as a maleimide, and a sulfhydryl group.

A “cell” as used herein, refers to a cell carrying out metabolic or other function sufficient to preserve or replicate its genomic DNA. A cell can be identified by well-known methods in the art including, for example, presence of an intact membrane, staining by a particular dye, ability to produce progeny or, in the case of a gamete, ability to combine with a second gamete to produce a viable offspring. Cells may include prokaryotic and eukaroytic cells. Prokaryotic cells include but are not limited to bacteria. Eukaryotic cells include but are not limited to yeast cells and cells derived from plants and animals, for example mammalian, insect (e.g., spodoptera) and human cells. Cells may be useful when they are naturally nonadherent or have been treated not to adhere to surfaces, for example by trypsinization.

“Nucleic acid” refers to deoxyribonucleotides or ribonucleotides and polymers thereof in either single- or double-stranded form, and complements thereof. The term “polynucleotide” refers to a linear sequence of nucleotides. The term “nucleotide” typically refers to a single unit of a polynucleotide, i.e., a monomer. Nucleotides can be ribonucleotides, deoxyribonucleotides, or modified versions thereof. Examples of polynucleotides contemplated herein include single and double stranded DNA, single and double stranded RNA (including siRNA), and hybrid molecules having mixtures of single and double stranded DNA and RNA. Nucleic acid as used herein also refers nucleic acids that have the same basic chemical structure as a naturally occurring nucleic acids. Such analogues have modified sugars and/or modified ring substituents, but retain the same basic chemical structure as the naturally occurring nucleic acid. A nucleic acid mimetic refers to chemical compounds that have a structure that is different the general chemical structure of a nucleic acid, but that functions in a manner similar to a naturally occurring nucleic acid. Examples of such analogues include, without limitation, phosphorothioates, phosphoramidates, methyl phosphonates, chiral-methyl phosphonates, 2-O-methyl ribonucleotides, and peptide-nucleic acids (PNAs).

A “detectable moiety” as used herein refers to a moiety that can be covalently or noncovalently attached to a compound or biomolecule that can be detected for instance, using techniques known in the art. In embodiments, the detectable moiety is covalently attached. The detection moiety may provide for imaging of the attached compound or biomolecule. The detection moiety may indicate the contacting between two compounds. Exemplary detectable moieties are fluorophores, antibodies, reactive dies, radio-labeled moieties, magnetic contrast agents, and quantum dots. Exemplary fluorophores include fluorescein, BODIPY®, and cyanine dyes. Exemplary radionuclides include Fluorine-18, Gallium-68, and Copper-64. Exemplary magnetic contrast agents include gadolinium, iron oxide and iron platinum, and manganese.

A “water soluble moiety” as used herein refers to any moiety that enhances the water solubility of the compound or molecule to which it is bound. A water soluble moiety may alter the partitioning coefficient of a compound or molecule to which it is bound thereby making the molecule more or less hydrophilic. The more hydrophobic a compound, the higher its partition constant. The more hydrophilic a compound, the lower its partition constant. In some embodiments, the water soluble groups can decrease the partition constant of precursor molecules (which have a higher partition constant before attachment of the water soluble group) at least by about 10%, 20%, 30%, 40%, 50%, 60%, 70%, 80%, or 90%. In some embodiments, the water soluble groups described herein can decrease the partition constant of precursor molecules by 1-fold, 2-fold, 3-fold, 4-fold, or greater. Exemplary water soluble moieties include moieties such as poly(oxyethylated polyols) such as poly(oxyethylated glycerol), poly(oxyethylated sorbitol), and poly(oxyethylated glucose); poly(vinyl alcohol) (“PVA”); dextran; carbohydrate-based polymers and the like (including linear chains or branched chains); polyethylene glycol moieties of formula

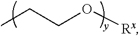

wherein y is an integer from 1 to 50 and R^{x} is —OH or —OMe; polyvinylpyrrolidone moieties; or poly 2-ethyl oxazoline moieties.

In some embodiments, the water soluble group can include a moiety containing a heteroatom (e.g., oxygen or nitrogen). In some embodiments to improve the water solubility of compounds herein a water soluble group is covalently attached at one or more positions. Such moieties include substituted alkyl moiety, substituted heteroalkyl moiety, substituted cycloalkyl moiety, substituted heteroalkyl moiety, or substituted aryl moiety. In embodiments, the moiety contains an alcohol moiety (an organic moiety having an —OH bound to a carbon atom), ester linker moiety (the linker moiety —C(O)O— between two carbon atoms), ether linker moiety (the linker moiety —O— between two carbon atoms), amine (—NH₂) moiety, nitrile (—CN) moiety, ketone moiety (the linker moiety —C(O)— between two carbon atoms), or aldehyde (—C(O)H) moiety.

### I. Methods

In a first aspect, there is provided a method for synthesizing a 3-substituted-6-alkenyl-1,2,4,5-tetrazine having the formula:

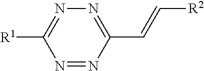

The method includes (i) contacting a first compound with structure of formula:

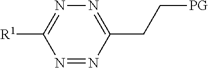

with a second compound of formula R²—X in a solvent system. The solvent system includes 1,2,3,4,5-pentaphenyl-1′-(di-tert-butylphosphino)ferrocene, Pd₂(dba)₃ and a base. The method further includes (ii) irradiating the solvent system with microwave radiation, thereby synthesizing the 3-substituted-6-alkenyl-1,2,4,5-tetrazine. R¹ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. R² is substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. X is a halogen. PG is a protecting group.

In one embodiment, R¹ is hydrogen. In one embodiment, R¹ is substituted or unsubstituted alkyl. In one embodiment, R¹ is unsubstituted alkyl. In one embodiment, R¹ is methyl. In one embodiment, R¹ is tert-butyl. In one embodiment, R¹ is substituted or unsubstituted heteroalkyl. In one embodiment, R¹ is alkylmethanesulfonate. In one embodiment, R¹ is —(CH₂)₂—NHBoc. In one embodiment, R¹ is substituted or unsubstituted aryl. In one embodiment, R¹ is unsubstituted aryl. In one embodiment, R¹ is phenyl. In one embodiment, R¹ is substituted or unsubstituted heteroaryl. In one embodiment, R¹ is unsubstituted heteroaryl. In one embodiment, R¹ is thiophene. In one embodiment, R¹ is

In one embodiment, R² is substituted or unsubstituted aryl. In one embodiment, R² is unsubstituted aryl. In one embodiment, R² is phenyl. In one embodiment, R² is substituted aryl. In one embodiment, R² is phenyl substituted with alkyl or alkyloxy. In one embodiment, R² is substituted or unsubstituted heteroaryl. In one embodiment, R² is substituted indole. In one embodiment, R² is a nucleic acid base, e.g., adenine, thymine, uridine, cytosine. In one embodiment, the nucleic acid base is protected, as known in the art. In one embodiment, R² is thiophenyl. In one embodiment, R² is quinolinyl. In one embodiment, R² is styryl. In one embodiment, R² is substituted or unsubstituted heterocycloalkyl. In one embodiment, R² is coumarinyl.

In one embodiment, the solvent system includes dimethylformamide (DMF).

In one embodiment, X is iodine or bromine.

In one embodiment, protecting group LG is mesylate.

In one embodiment, the solvent system is irradiated with microwave radiation for a suitable time to afford the 3-substituted-6-alkenyl-1,2,4,5-tetrazine, e.g., 5, 10, 15, 20, 25, 30, 35, 40, 45, 50 minutes, or longer. In one embodiment, the microwave irradiation provides a constant reaction temperature. In one embodiment, the constant reaction temperature is 30° C., 35° C., 40° C., 45° C., 50° C., 55° C., 60° C., 65° C., 70° C., 75° C., 80° C., 85° C., 90° C., 95° C., or even greater.

In one embodiment, the base is an amine-containing base. In one embodiment, the base is triethylamine or dicyclohexylmethylamine.

In another aspect, there is provided a method of synthesizing a compound of Formula I:

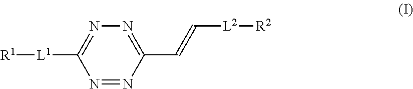

the method including reacting a compound of Formula II:

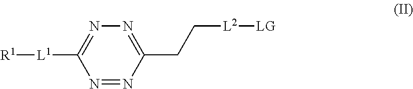

with a compound of Formula III:

R²—X (III),

The compounds of Formula II and III are reacted in the presence of a Pd catalyst and a ligand under basic conditions.

In embodiments, the method includes isolating an compound of Formula IIa as described herein.

In the compounds of Formula I and II, L¹ and L² are independently a bond or a covalent linker. R¹ is hydrogen, halogen, —CN, —N₃, —CF₃, —CCl₃, —CBr₃, —CI₃, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a biomolecule, a dye or fluorophore. In embodiments, R¹ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a biomolecule, a dye or fluorophore. R² is substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl or a biomolecule, a dye or fluorophore. In the compound of Formula II, LG is a leaving group. In the compound of Formula III, X is halogen.

In embodiments, LG is hydrogen, halogen, —CN, —N₃, —CF₃, —CCl₃, —CBr₃, —CI₃, —OH, —NH₂, —COOH, —CONH₂, —NO₂, —SH, —SO₃H, —SO₄H, —SO₂NH₂, —NNH₂, —ONH₂, —NHC(O)NHNH₂, —NHC(O)NH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, R²⁶-substituted or unsubstituted aryloxy, R²⁶-substituted or unsubstituted heteroaryloxy, R²⁶-substituted or unsubstituted alkyl (e.g. C₁-C₈ alkyl, C₁-C₆ alkyl, or C₁-C₄ alkyl), R²⁶-substituted or unsubstituted heteroalkyl (e.g. 2 to 8 membered heteroalkyl, 2 to 6 membered heteroalkyl, or 2 to 4 membered heteroalkyl), R²⁶-substituted or unsubstituted cycloalkyl (e.g. C₃-C₈ cycloalkyl, C₄-C₈ cycloalkyl, or C₅-C₆ cycloalkyl), R²⁶-substituted or unsubstituted heterocycloalkyl (e.g. 3 to 8 membered heterocycloalkyl, 4 to 8 membered heterocycloalkyl, or 5 to 6 membered heterocycloalkyl), R²⁶-substituted or unsubstituted aryl (e.g. C₆-C₁₀ aryl or C₆ aryl), or R²⁶-substituted or unsubstituted heteroaryl (e.g. 5 to 10 membered heteroaryl, 5 to 9 membered heteroaryl, or 5 to 6 membered heteroaryl).

In embodiments, LG is an electron withdrawing group. In embodiments, LG is —C(O)NR^{3A}R^{3B}, —NO₂, —CN, —SO₂R^{3A}, —C(O)R^{3A}, —C(O)Cl, CF₃, N₂⁺, —OR^{3A}₂⁺, —OSO₂R^{3A}F, a perfluoroalkylsulfonate, a tosylate, a mesylate, a halogen, —OH₂⁺, —ONO₂, —OPO(OH)₂, —ONO₂, —S(R^{3B}R^{3C})⁺, —N(R^{3A}R^{3B}R^{3C})⁺ or —OCOR^{3A}.

R^{3A}, R^{3B} and R^{3C} are independently hydrogen, halogen, —CN, —N₃, —CF₃, —CCl₃, —CBr₃, —CI₃, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCCl₃, —OCBr₃, —OCI₃, —OCHF₂, —OCHC1₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3B} and R^{3c} substituents bonded to the same nitrogen atom may optionally be joined to form a substituted or unsubstituted heterocycloalkyl or substituted or unsubstituted heteroaryl.

In embodiments, R^{3A} is hydrogen, halogen, —CN, —N₃, —CF₃, —CCl₃, —CBr₃, —CI₃, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, R^{3D}-substituted or unsubstituted alkyl (e.g. C₁-C₈ alkyl, C₁-C₆ alkyl, or C₁-C₄ alkyl), R^{3D}-substituted or unsubstituted heteroalkyl (e.g. 2 to 8 membered heteroalkyl, 2 to 6 membered heteroalkyl, or 2 to 4 membered heteroalkyl), R^{3D}-substituted or unsubstituted cycloalkyl (e.g. C₃-C₈ cycloalkyl, C₄-C₈ cycloalkyl, or C₅-C₆ cycloalkyl), R^{3D}-substituted or unsubstituted heterocycloalkyl (e.g. 3 to 8 membered heterocycloalkyl, 4 to 8 membered heterocycloalkyl, or 5 to 6 membered heterocycloalkyl), R^{3D}-substituted or unsubstituted aryl (e.g. C₆-C₁₀ aryl or C₆ aryl), or R^{3D}-substituted or unsubstituted heteroaryl (e.g. 5 to 10 membered heteroaryl, 5 to 9 membered heteroaryl, or 5 to 6 membered heteroaryl).

R^{3D} is independently hydrogen, halogen, —CN, —N₃, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, R^{3E}-substituted or unsubstituted alkyl (e.g. C₁-C₈ alkyl, C₁-C₆ alkyl, or C₁-C₄ alkyl), R^{3E}-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R^{3E}-substituted or unsubstituted cycloalkyl (e.g. C₃-C₈ cycloalkyl, C₄-C₈ cycloalkyl, or C₅-C₆ cycloalkyl), R^{3E}-substituted or unsubstituted heterocycloalkyl (e.g. 3 to 8 membered heterocycloalkyl, 4 to 8 membered heterocycloalkyl, or 5 to 6 membered heterocycloalkyl), R^{3E}-substituted or unsubstituted aryl (e.g. C₆-C₁₀ aryl or C₆ aryl), or R^{3E}-substituted or unsubstituted heteroaryl (e.g. 5 to 10 membered heteroaryl, 5 to 9 membered heteroaryl, or 5 to 6 membered heteroaryl).

In embodiments, R^{3B} is hydrogen, halogen, —CN, —N₃, —CF₃, —CCl₃, —CBr₃, —CI₃, —OH, —NH₂, —COOH, —CONH₂, —NO₂, —SH, —SO₃H, —SO₄H, —SO₂NH₂, —NNH₂, —ONH₂, —NHC(O)NHNH₂, —NHC(O)NH₂, —NHSO₂H, —NHC(O)H, —NHC(O)—OH, —NHOH, —OCF₃, —OCCl₃, —OCBr₃, —OCI₃, —OCHF₂, —OCHCl₂, —OCHBr₂, —OCHI₂, R^{3F}-substituted or unsubstituted alkyl (e.g. C₁-C₈ alkyl, C₁-C₆ alkyl, or C₁-C₄ alkyl), R^{3F}-substituted or unsubstituted heteroalkyl (e.g. 2 to 8 membered heteroalkyl, 2 to 6 membered heteroalkyl, or 2 to 4 membered heteroalkyl), R^{3F}-substituted or unsubstituted cycloalkyl (e.g. C₃-C₈ cycloalkyl, C₄-C₈ cycloalkyl, or C₅-C₆ cycloalkyl), R^{3F}-substituted or unsubstituted heterocycloalkyl (e.g. 3 to 8 membered heterocycloalkyl, 4 to 8 membered heterocycloalkyl, or 5 to 6 membered heterocycloalkyl), R^{3F}-substituted or unsubstituted aryl (e.g. C₆-C₁₀ aryl or C₆ aryl), or R^{3F}-substituted or unsubstituted heteroaryl (e.g. 5 to 10 membered heteroaryl, 5 to 9 membered heteroaryl, or 5 to 6 membered heteroaryl). In embodiments, R^{3B} substituents bonded to the same nitrogen atom may optionally be joined to form a R^{3F}-substituted or unsubstituted heterocycloalkyl or R^{3F}-substituted or unsubstituted heteroaryl.

R^{3F} is independently hydrogen, halogen, —CF₃, —CN, —N₃, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, R^{3G}-substituted or unsubstituted alkyl (e.g. C₁-C₈ alkyl, C₁-C₆ alkyl, or C₁-C₄ alkyl), R^{3G}-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R^{3G}-substituted or unsubstituted cycloalkyl (e.g. C₃-C₈ cycloalkyl, C₄-C₈ cycloalkyl, or C₅-C₆ cycloalkyl), R^{3G}-substituted or unsubstituted heterocycloalkyl (e.g. 3 to 8 membered heterocycloalkyl, 4 to 8 membered heterocycloalkyl, or 5 to 6 membered heterocycloalkyl), R^{3G}-substituted or unsubstituted aryl (e.g. C₆-C₁₀ aryl or C₆ aryl), or R^{3G}-substituted or unsubstituted heteroaryl (e.g. 5 to 10 membered heteroaryl, 5 to 9 membered heteroaryl, or 5 to 6 membered heteroaryl).

In embodiments, R^{3C} is hydrogen, halogen, —CN, —N₃, —CF₃, —CCl₃, —CBr₃, —CI₃, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCCl₃, —OCBr₃, —OCI₃, —OCHF₂, —OCHC1₂, —OCHBr₂, —OCHI₂, R^{3H}-substituted or unsubstituted alkyl (e.g. C₁-C₈ alkyl, C₁-C₆ alkyl, or C₁-C₄ alkyl), R^{3H}-substituted or unsubstituted heteroalkyl (e.g. 2 to 8 membered heteroalkyl, 2 to 6 membered heteroalkyl, or 2 to 4 membered heteroalkyl), R^{3H}-substituted or unsubstituted cycloalkyl (e.g. C₃-C₈ cycloalkyl, C₄-C₈ cycloalkyl, or C₅-C₆ cycloalkyl), R^{3H}-substituted or unsubstituted heterocycloalkyl (e.g. 3 to 8 membered heterocycloalkyl, 4 to 8 membered heterocycloalkyl, or 5 to 6 membered heterocycloalkyl), R^{3H}-substituted or unsubstituted aryl (e.g. C₆-C₁₀ aryl or C₆ aryl), or R^{3H}-substituted or unsubstituted heteroaryl (e.g. 5 to 10 membered heteroaryl, 5 to 9 membered heteroaryl, or 5 to 6 membered heteroaryl). In embodiments, R^{3C} substituents bonded to the same nitrogen atom may optionally be joined to form a R^{3H}-substituted or unsubstituted heterocycloalkyl or R^{3H}-substituted or unsubstituted heteroaryl.

R^{3H} is independently hydrogen, halogen, —N₃, —CF₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O)NH₂, —NHSO₂H, —NHC═(O)H, —NHC(O)OH, —NHOH, —OCF₃, —OCHF₂, R³-substituted or unsubstituted alkyl (e.g. C₁-C₈ alkyl, C₁-C₆ alkyl, or C₁-C₄ alkyl), R³-substituted or unsubstituted heteroalkyl (e.g. 2 to 10 membered heteroalkyl, 2 to 8 membered heteroalkyl, 4 to 8 membered heteroalkyl, 2 to 6 membered heteroalkyl, or 2 to 4 membered heteroalkyl), R^{3I}-substituted or unsubstituted cycloalkyl (e.g. C₃-C₈ cycloalkyl, C₄-C₈ cycloalkyl, or C₅-C₆ cycloalkyl), R³-substituted or unsubstituted heterocycloalkyl (e.g. 3 to 8 membered heterocycloalkyl, 4 to 8 membered heterocycloalkyl, or 5 to 6 membered heterocycloalkyl), R³-substituted or unsubstituted aryl (e.g. C₆-C₁₀ aryl or C₆ aryl), or R^{3I}-substituted or unsubstituted heteroaryl (e.g. 5 to 10 membered heteroaryl, 5 to 9 membered heteroaryl, or 5 to 6 membered heteroaryl).

R^{3E}, R^{3G} and R^{3I} are independently oxo, halogen, —F, —Cl, —Br, —I, —CF₃, —CCl₃, —N₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl (e.g. C₁-C₈ alkyl, C₁-C₆ alkyl, or C₁-C₄ alkyl), unsubstituted heteroalkyl (e.g. 2 to 8 membered heteroalkyl, 2 to 6 membered heteroalkyl, or 2 to 4 membered heteroalkyl), unsubstituted cycloalkyl (e.g. C₃-C₈ cycloalkyl, C₄-C₈ cycloalkyl, or C₅-C₆ cycloalkyl), unsubstituted heterocycloalkyl (e.g. 3 to 8 membered heterocycloalkyl, 4 to 8 membered heterocycloalkyl, or 5 to 6 membered heterocycloalkyl), unsubstituted aryl (e.g. C₆-C₁₀ aryl or C₆ aryl), or unsubstituted heteroaryl (e.g. 5 to 10 membered heteroaryl, 5 to 9 membered heteroaryl, or 5 to 6 membered heteroaryl).

In embodiments, there is provided a compound having the formula:

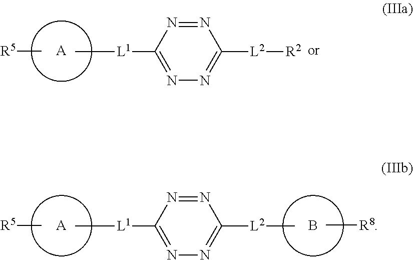

L¹, L², R² and R⁵ are as described herein, including embodiments. Ring A is R⁵-substituted or unsubstituted cycloalkyl (e.g. C₃-C₈ cycloalkyl, C₄-C₈ cycloalkyl, or C₅-C₆ cycloalkyl), R⁵-substituted or unsubstituted heterocycloalkyl (e.g. 3 to 8 membered heterocycloalkyl, 4 to 8 membered heterocycloalkyl, or 5 to 6 membered heterocycloalkyl), R⁵-substituted or unsubstituted aryl (e.g. C₆-C₁₀ aryl or C₆ aryl), or R⁵-substituted or unsubstituted heteroaryl (e.g. 5 to 10 membered heteroaryl, 5 to 9 membered heteroaryl, or 5 to 6 membered heteroaryl). In embodiments, Ring A is R⁵-substituted or unsubstituted aryl (e.g. C₆-C₁₀ aryl or C₆ aryl), or R⁵-substituted or unsubstituted heteroaryl (e.g. 5 to 10 membered heteroaryl, 5 to 9 membered heteroaryl, or 5 to 6 membered heteroaryl). In embodiments, Ring A is unsubstituted aryl (e.g. C₆-C₁₀ aryl or C₆ aryl), or unsubstituted heteroaryl (e.g. 5 to 10 membered heteroaryl, 5 to 9 membered heteroaryl, or 5 to 6 membered heteroaryl).

In embodiments, Ring B is R-substituted or unsubstituted cycloalkyl (e.g. C₃-C₈ cycloalkyl, C₄-C₈ cycloalkyl, or C₅-C₆ cycloalkyl), R⁸-substituted or unsubstituted heterocycloalkyl (e.g. 3 to 8 membered heterocycloalkyl, 4 to 8 membered heterocycloalkyl, or 5 to 6 membered heterocycloalkyl), R⁸-substituted or unsubstituted aryl (e.g. C₆-C₁₀ aryl or C₆ aryl), or R⁸-substituted or unsubstituted heteroaryl (e.g. 5 to 10 membered heteroaryl, 5 to 9 membered heteroaryl, or 5 to 6 membered heteroaryl). In embodiments, Ring A is R⁸-substituted or unsubstituted aryl (e.g. C₆-C₁₀ aryl or C₆ aryl), or R⁸-substituted or unsubstituted heteroaryl (e.g. 5 to 10 membered heteroaryl, 5 to 9 membered heteroaryl, or 5 to 6 membered heteroaryl). In embodiments, Ring B is unsubstituted aryl (e.g. C₆-C₁₀ aryl or C₆ aryl), or unsubstituted heteroaryl (e.g. 5 to 10 membered heteroaryl, 5 to 9 membered heteroaryl, or 5 to 6 membered heteroaryl).

In embodiments, there is provided a compound having the formula

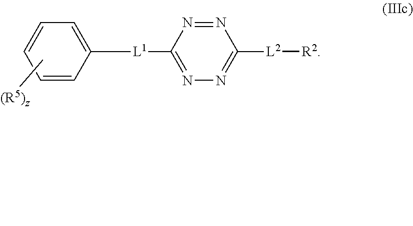

L¹, L², R² and R⁵ are as described herein, including embodiments. The symbol z is an integer from 0 to 5. In embodiments, z is 0. In embodiments, z is 1. In embodiments, z is 2. In embodiments, z is 3. In embodiments, z is 4. In embodiments, z is 5.

In embodiments, R¹ is hydrogen, R⁵-substituted or unsubstituted alkyl (e.g. C₁-C₈alkyl, C₁-C₆ alkyl, or C₁-C₄ alkyl), R⁵-substituted or unsubstituted heteroalkyl (e.g. 2 to 8 membered heteroalkyl, 2 to 6 membered heteroalkyl, or 2 to 4 membered heteroalkyl), R⁵-substituted or unsubstituted cycloalkyl (e.g. C₃-C₈ cycloalkyl, C₄-C₈ cycloalkyl, or C₅-C₆ cycloalkyl), R⁵-substituted or unsubstituted heterocycloalkyl (e.g. 3 to 8 membered heterocycloalkyl, 4 to 8 membered heterocycloalkyl, or 5 to 6 membered heterocycloalkyl), R⁵-substituted or unsubstituted aryl (e.g. C₆-C₁₀ aryl or C₆ aryl), or R⁵-substituted or unsubstituted heteroaryl (e.g. 5 to 10 membered heteroaryl, 5 to 9 membered heteroaryl, or 5 to 6 membered heteroaryl). In embodiments, R¹ is a biomolecule. In embodiments, R¹ is a fluorophore.

R⁵ is independently oxo, halogen, —CF₃, —N₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R⁶-substituted or unsubstituted alkyl (e.g. C₁-C₈ alkyl, C₁-C₆ alkyl, or C₁-C₄ alkyl), R⁶-substituted or unsubstituted heteroalkyl (e.g. 2 to 8 membered heteroalkyl, 2 to 6 membered heteroalkyl, or 2 to 4 membered heteroalkyl), R⁶-substituted or unsubstituted cycloalkyl (e.g. C₃-C₈ cycloalkyl, C₄-C₈ cycloalkyl, or C₅-C₆ cycloalkyl), R⁶-substituted or unsubstituted heterocycloalkyl (e.g. 3 to 8 membered heterocycloalkyl, 4 to 8 membered heterocycloalkyl, or 5 to 6 membered heterocycloalkyl), R⁶-substituted or unsubstituted aryl (e.g. C₆-C₁₀ aryl or C₆ aryl), or R⁶-substituted or unsubstituted heteroaryl (e.g. 5 to 10 membered heteroaryl, 5 to 9 membered heteroaryl, or 5 to 6 membered heteroaryl). In embodiments, R⁵ is 2,7-difluoro-6-hydroxy-9-yl-3H-xanthen-3-one or 3-(dimethyl-14-azanylidene)-N,N,methyl-9-yl-3H-xanthen-6-amine.

R⁶ is independently oxo, halogen, —CF₃, —N₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R⁷-substituted or unsubstituted alkyl (e.g. C₁-C₈ alkyl, C₁-C₆ alkyl, or C₁-C₄ alkyl), R⁷-substituted or unsubstituted heteroalkyl (e.g. 2 to 8 membered heteroalkyl, 2 to 6 membered heteroalkyl, or 2 to 4 membered heteroalkyl), R⁷-substituted or unsubstituted cycloalkyl (e.g. C₃-C₈ cycloalkyl, C₄-C₈ cycloalkyl, or C₅-C₆ cycloalkyl), R⁷-substituted or unsubstituted heterocycloalkyl (e.g. 3 to 8 membered heterocycloalkyl, 4 to 8 membered heterocycloalkyl, or 5 to 6 membered heterocycloalkyl), R⁷-substituted or unsubstituted aryl (e.g. C₆-C₁₀ aryl or C₆ aryl), or R⁷-substituted or unsubstituted heteroaryl (e.g. 5 to 10 membered heteroaryl, 5 to 9 membered heteroaryl, or 5 to 6 membered heteroaryl).

In embodiments, R² is hydrogen, R⁸-substituted or unsubstituted alkyl (e.g. C₁-C₈ alkyl, C₁-C₆ alkyl, or C₁-C₄ alkyl), R⁸-substituted or unsubstituted heteroalkyl (e.g. 2 to 8 membered heteroalkyl, 2 to 6 membered heteroalkyl, or 2 to 4 membered heteroalkyl), R⁸-substituted or unsubstituted cycloalkyl (e.g. C₃-C₈ cycloalkyl, C₄-C₈ cycloalkyl, or C₅-C₆ cycloalkyl), R⁸-substituted or unsubstituted heterocycloalkyl (e.g. 3 to 8 membered heterocycloalkyl, 4 to 8 membered heterocycloalkyl, or 5 to 6 membered heterocycloalkyl), R⁸-substituted or unsubstituted aryl (e.g. C₆-C₁₀ aryl or C₆ aryl), or R⁸-substituted or unsubstituted heteroaryl (e.g. 5 to 10 membered heteroaryl, 5 to 9 membered heteroaryl, or 5 to 6 membered heteroaryl). In embodiments, R² is a biomolecule. In embodiments, R² is a fluorophore.

R⁸ is independently oxo, halogen, —CF₃, —N₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R⁹-substituted or unsubstituted alkyl (e.g. C₁-C₈ alkyl, C₁-C₆ alkyl, or C₁-C₄ alkyl), R⁹-substituted or unsubstituted heteroalkyl (e.g. 2 to 8 membered heteroalkyl, 2 to 6 membered heteroalkyl, or 2 to 4 membered heteroalkyl), R⁹-substituted or unsubstituted cycloalkyl (e.g. C₃-C₈ cycloalkyl, C₄-C₈ cycloalkyl, or C₅-C₆ cycloalkyl), R⁹-substituted or unsubstituted heterocycloalkyl (e.g. 3 to 8 membered heterocycloalkyl, 4 to 8 membered heterocycloalkyl, or 5 to 6 membered heterocycloalkyl), R⁹-substituted or unsubstituted aryl (e.g. C₆-C₁₀ aryl or C₆ aryl), or R⁹-substituted or unsubstituted heteroaryl (e.g. 5 to 10 membered heteroaryl, 5 to 9 membered heteroaryl, or 5 to 6 membered heteroaryl).

R⁹ is independently oxo, halogen, —CF₃, —N₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, R¹⁰-substituted or unsubstituted alkyl (e.g. C₁-C₈ alkyl, C₁-C₆ alkyl, or C₁-C₄ alkyl), R¹⁰-substituted or unsubstituted heteroalkyl (e.g. 2 to 8 membered heteroalkyl, 2 to 6 membered heteroalkyl, or 2 to 4 membered heteroalkyl), R¹⁰-substituted or unsubstituted cycloalkyl (e.g. C₃-C₈ cycloalkyl, C₄-C₅cycloalkyl, or C₅-C₆ cycloalkyl), R¹⁰-substituted or unsubstituted heterocycloalkyl (e.g. 3 to 8 membered heterocycloalkyl, 4 to 8 membered heterocycloalkyl, or 5 to 6 membered heterocycloalkyl), R¹⁰-substituted or unsubstituted aryl (e.g. C₆-C₁₀ aryl or C₆ aryl), or R¹⁰-substituted or unsubstituted heteroaryl (e.g. 5 to 10 membered heteroaryl, 5 to 9 membered heteroaryl, or 5 to 6 membered heteroaryl).

R⁷, R¹⁰, R¹¹, R¹², R¹⁴, R¹⁵, R¹⁶, R¹⁷, R¹⁸, R¹⁹, R²⁰, R²¹, R¹⁶, R²², R²³, R²⁴, R²⁵ and R²⁶ are independently oxo, halogen, —F,

—Cl, —Br, —I, —CF₃, —CCl₃, —N₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl (e.g. C₁-C₈ alkyl, C₁-C₆ alkyl, or C₁-C₄ alkyl), unsubstituted heteroalkyl (e.g. 2 to 8 membered heteroalkyl, 2 to 6 membered heteroalkyl, or 2 to 4 membered heteroalkyl), unsubstituted cycloalkyl (e.g. C₃-C₈ cycloalkyl, C₄-C₈ cycloalkyl, or C₅-C₆ cycloalkyl), unsubstituted heterocycloalkyl (e.g. 3 to 8 membered heterocycloalkyl, 4 to 8 membered heterocycloalkyl, or 5 to 6 membered heterocycloalkyl), unsubstituted aryl (e.g. C₆-C₁₀ aryl or C₆ aryl), or unsubstituted heteroaryl (e.g. 5 to 10 membered heteroaryl, 5 to 9 membered heteroaryl, or 5 to 6 membered heteroaryl).

In embodiments, L¹ is independently a bond, —NR^{1C}—, —O—, —S—, —C(O)—, —S(O)—, —S(O)₂—, —C(O)—, —C(O)N(R^{1C})—, —N(R^{1C})S(O)—, —N(R^{1C})S(O)₂—, —C(O)OR^{1C}, —CH(R^{1A})N(C(O)OR^{1C})—, —CH(R^{1A})N(C(O)R^{1C})—, —CH(R^{1A})N(SO₂R^{1C})—, —CH(R^{1A})N(R^{1C})—, —CH(R^{1A})C(O)N(R^{1C})—, —CH(R^{1A})N(R^{1C})C(O)—, —CH(R^{1A})N(R^{1C})S(O)—, —CH(R^{1A})N(R^{1C})S(O)₂—, —N(R^{1C})C(O)N(R^{1D})—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

In embodiments, L¹ is substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. In embodiments, L¹ is R¹¹-substituted or unsubstituted alkylene (e.g. C₁-C₈ alkylene, C₁-C₆ alkylene, or C₁-C₄ alkylene), R¹¹-substituted or unsubstituted heteroalkylene (e.g. 2 to 8 membered heteroalkylene, 2 to 6 membered heteroalkylene, or 2 to 4 membered heteroalkylene), R¹¹-substituted or unsubstituted cycloalkylene (e.g. C₃-C₈ cycloalkylene, C₄-C₈ cycloalkylene, or C₅-C₆ cycloalkylene), R¹¹-substituted or unsubstituted heterocycloalkylene (e.g. 3 to 8 membered heterocycloalkylene, 4 to 8 membered heterocycloalkylene, or 5 to 6 membered heterocycloalkylene), R″¹-substituted or unsubstituted arylene (e.g. C₆-C₁₀ arylene or C₆ arylene), or R¹¹-substituted or unsubstituted heteroarylene (e.g. 5 to 10 membered heteroarylene, 5 to 9 membered heteroarylene, or 5 to 6 membered heteroarylene). In embodiments, L¹ is a bond. In some embodiments, L¹ is —NR^{1C}—. In some embodiments, L¹ is —O—. In some embodiments, L¹ is —S—. In some embodiments, L¹ is —C(O)—. In some embodiments, L¹ is —S(O)—. In some embodiments, L¹ is —S(O)₂—. In some embodiments, L¹ is independently —CR^{1A}R^{1B}—. In some embodiments, L¹ is independently

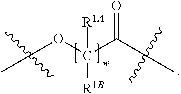

In some embodiments, L¹ is independently

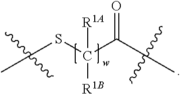

In some embodiments, L¹ is independently

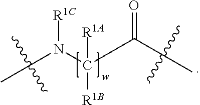

In embodiments, L² is independently a bond, —NR^{2C}—, —O—, —S—, —C(O)—, —S(O)—, —S(O)₂—, —C(O)—, —C(O)N(R^{2C})—, —N(R^{2C})S(O)—, —N(R^{2C})S(O)₂—, —C(O)OR^{2C}, —CH(R^{2A})N(C(O)OR^{2C})—, —CH(R^{2A})N(C(O)R^{2C})—, —CH(R^{2A})N(SO₂R^{2C})—, —CH(R^{2A})N(R^{2C})—, —CH(R^{2A})C(O)N(R^{2C})—, —CH(R^{2A})N(R^{2C})C(O)—, —CH(R^{2A})N(R^{2C})S(O)—, —CH(R^{2A})N(R^{2C})S(O)₂—, —N(R^{2C})C(O)N(R^{2D})—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene.

In embodiments, L² is substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. In embodiments, L² is R¹²-substituted or unsubstituted alkylene (e.g. C₁-C₈ alkylene, C₁-C₆ alkylene, or C₁-C₄ alkylene), R¹²-substituted or unsubstituted heteroalkylene (e.g. 2 to 8 membered heteroalkylene, 2 to 6 membered heteroalkylene, or 2 to 4 membered heteroalkylene), R¹²-substituted or unsubstituted cycloalkylene (e.g. C₃-C₈ cycloalkylene, C₄-C₈ cycloalkylene, or C₅-C₆ cycloalkylene), R¹²-substituted or unsubstituted heterocycloalkylene (e.g. 3 to 8 membered heterocycloalkylene, 4 to 8 membered heterocycloalkylene, or 5 to 6 membered heterocycloalkylene), R¹²-substituted or unsubstituted arylene (e.g. C₆-C₁₀ arylene or C₆ arylene), or R¹²-substituted or unsubstituted heteroarylene (e.g. 5 to 10 membered heteroarylene, 5 to 9 membered heteroarylene, or 5 to 6 membered heteroarylene). In some embodiments, L² is —NR²C—. In some embodiments, L² is —O—. In some embodiments, L² is —S—. In some embodiments, L² is —C(O)—. In some embodiments, L² is —S(O)—. In some embodiments, L² is —S(O)₂—. In some embodiments, L² is independently —CR^{2A}R^{2B}—. In some embodiments, L² is independently

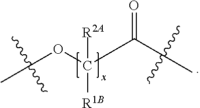

In some embodiments, L² is independently

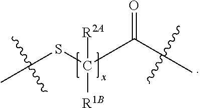

In some embodiments, L² is independently

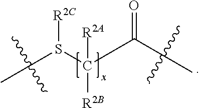

R^{1A}, R^{1B}, R^{2A}, R^{2B}, R^{4A} and R^{4B} are independently hydrogen, oxo, halogen, —CX^{a}₃, —N₃, —CN, —SOₙ₁R^{10a}, —SOᵥ₁NR^{7a}R^{8a}, —NHNH₂, —ONR^{7a}R^{8a}, —NHC═(O)NHNH₂, —NHC═(O)NR^{7a}R^{8a}, —N(O)ₘ₁, —NR^{7a}R^{8a}, —C(O)R^{9a}, —C(O)—OR^{9a}, —C(O)NR^{7a}R^{8a}, —OR^{10a}—NR^{7a}SO₂R^{10a}, —NR^{7a}C═(O)R^{9a}, —NR^{7a}C(O)—OR^{9a}, —NR^{7a}OR^{9a}, —OCX^{a}₃, —OCHX^{a}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl. R^{1A}, R^{1B}, R^{2A}, R^{2B}, R^{4A} and R^{4B} substituents bonded to the same atom may optionally be joined to form a substituted or unsubstituted cycloalkyl or substituted or unsubstituted heterocycloalkyl.

R^{7a}, R^{8a}, R^{9a} and R^{10a} are independently hydrogen, halogen, —CF₃, —N₃, —CN, —OH, —NH₂,

—COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl. R^{7a} and R^{8a} substituents bonded to the same nitrogen atom may optionally be joined to form an unsubstituted heterocycloalkyl or unsubstituted heteroaryl.

In embodiments, R^{1A} is R¹⁴-substituted or unsubstituted alkyl (e.g. C₁-C₈ alkyl, C₁-C₆ alkyl, or C₁-C₄ alkyl), R¹⁴-substituted or unsubstituted heteroalkyl (e.g. 2 to 8 membered heteroalkyl, 2 to 6 membered heteroalkyl, or 2 to 4 membered heteroalkyl), R¹⁴-substituted or unsubstituted cycloalkyl (e.g. C₃-C₈ cycloalkyl, C₄-C₈ cycloalkyl, or C₅-C₆ cycloalkyl), R¹⁴-substituted or unsubstituted heterocycloalkyl (e.g. 3 to 8 membered heterocycloalkyl, 4 to 8 membered heterocycloalkyl, or 5 to 6 membered heterocycloalkyl), R¹⁴-substituted or unsubstituted aryl (e.g. C₆-C₁₀ aryl or C₆ aryl), or R¹⁴-substituted or unsubstituted heteroaryl (e.g. 5 to 10 membered heteroaryl, 5 to 9 membered heteroaryl, or 5 to 6 membered heteroaryl).

In embodiments, R^{1B} is R¹⁵-substituted or unsubstituted alkyl (e.g. C₁-C₈ alkyl, C₁-C₆ alkyl, or C₁-C₄ alkyl), R¹⁵-substituted or unsubstituted heteroalkyl (e.g. 2 to 8 membered heteroalkyl, 2 to 6 membered heteroalkyl, or 2 to 4 membered heteroalkyl), R¹⁵-substituted or unsubstituted cycloalkyl (e.g. C₃-C₈ cycloalkyl, C₄-C₈ cycloalkyl, or C₅-C₆ cycloalkyl), R¹⁵-substituted or unsubstituted heterocycloalkyl (e.g. 3 to 8 membered heterocycloalkyl, 4 to 8 membered heterocycloalkyl, or 5 to 6 membered heterocycloalkyl), R¹⁵-substituted or unsubstituted aryl (e.g. C₆-C₁₀ aryl or C₆ aryl), or R¹⁵-substituted or unsubstituted heteroaryl (e.g. 5 to 10 membered heteroaryl, 5 to 9 membered heteroaryl, or 5 to 6 membered heteroaryl).

In embodiments, R^{2A} is R¹⁶-substituted or unsubstituted alkyl (e.g. C₁-C₈ alkyl, C₁-C₆ alkyl, or C₁-C₄ alkyl), R¹⁶-substituted or unsubstituted heteroalkyl (e.g. 2 to 8 membered heteroalkyl, 2 to 6 membered heteroalkyl, or 2 to 4 membered heteroalkyl), R¹⁶-substituted or unsubstituted cycloalkyl (e.g. C₃-C₈ cycloalkyl, C₄-C₈ cycloalkyl, or C₅-C₆ cycloalkyl), R¹⁶-substituted or unsubstituted heterocycloalkyl (e.g. 3 to 8 membered heterocycloalkyl, 4 to 8 membered heterocycloalkyl, or 5 to 6 membered heterocycloalkyl), R¹⁶-substituted or unsubstituted aryl (e.g. C₆-C₁₀ aryl or C₆ aryl), or R¹⁶-substituted or unsubstituted heteroaryl (e.g. 5 to 10 membered heteroaryl, 5 to 9 membered heteroaryl, or 5 to 6 membered heteroaryl).

In embodiments, R^{2B} is R¹⁷-substituted or unsubstituted alkyl (e.g. C₁-C₈ alkyl, C₁-C₆ alkyl, or C₁-C₄ alkyl), R¹⁷-substituted or unsubstituted heteroalkyl (e.g. 2 to 8 membered heteroalkyl, 2 to 6 membered heteroalkyl, or 2 to 4 membered heteroalkyl), R¹⁷-substituted or unsubstituted cycloalkyl (e.g. C₃-C₈ cycloalkyl, C₄-C₈ cycloalkyl, or C₅-C₆ cycloalkyl), R¹⁷-substituted or unsubstituted heterocycloalkyl (e.g. 3 to 8 membered heterocycloalkyl, 4 to 8 membered heterocycloalkyl, or 5 to 6 membered heterocycloalkyl), R¹⁷-substituted or unsubstituted aryl (e.g. C₆-C₁₀ aryl or C₆ aryl), or R¹⁷-substituted or unsubstituted heteroaryl (e.g. 5 to 10 membered heteroaryl, 5 to 9 membered heteroaryl, or 5 to 6 membered heteroaryl).

In embodiments, R^{4A} is R¹⁸-substituted or unsubstituted alkyl (e.g. C₁-C₈ alkyl, C₁-C₆ alkyl, or C₁-C₄ alkyl), R¹⁸-substituted or unsubstituted heteroalkyl (e.g. 2 to 8 membered heteroalkyl, 2 to 6 membered heteroalkyl, or 2 to 4 membered heteroalkyl), R¹⁸-substituted or unsubstituted cycloalkyl (e.g. C₃-C₈ cycloalkyl, C₄-C₈ cycloalkyl, or C₅-C₆ cycloalkyl), R¹⁸-substituted or unsubstituted heterocycloalkyl (e.g. 3 to 8 membered heterocycloalkyl, 4 to 8 membered heterocycloalkyl, or 5 to 6 membered heterocycloalkyl), R¹⁸-substituted or unsubstituted aryl (e.g. C₆-C₁₀ aryl or C₆ aryl), or R¹⁸-substituted or unsubstituted heteroaryl (e.g. 5 to 10 membered heteroaryl, 5 to 9 membered heteroaryl, or 5 to 6 membered heteroaryl).

In embodiments, R^{4B} is R¹⁹-substituted or unsubstituted alkyl (e.g. C₁-C₈ alkyl, C₁-C₆ alkyl, or C₁-C₄ alkyl), R¹⁹-substituted or unsubstituted heteroalkyl (e.g. 2 to 8 membered heteroalkyl, 2 to 6 membered heteroalkyl, or 2 to 4 membered heteroalkyl), R¹⁹-substituted or unsubstituted cycloalkyl (e.g. C₃-C₈ cycloalkyl, C₄-C₈ cycloalkyl, or C₅-C₆ cycloalkyl), R¹⁹-substituted or unsubstituted heterocycloalkyl (e.g. 3 to 8 membered heterocycloalkyl, 4 to 8 membered heterocycloalkyl, or 5 to 6 membered heterocycloalkyl), R¹⁹-substituted or unsubstituted aryl (e.g. C₆-C₁₀ aryl or C₆ aryl), or R¹⁹-substituted or unsubstituted heteroaryl (e.g. 5 to 10 membered heteroaryl, 5 to 9 membered heteroaryl, or 5 to 6 membered heteroaryl).

R^{1C}, R^{1D}, R^{2C}, R^{2D}, R^{4C} and R^{4D} are independently hydrogen, oxo, halogen, —CX₃, —N₃, —CN, —SO₂Cl, —SOₙ₃R^{10c}, —SOᵥ₃NR^{7c}R^{8c}, —NHNH₂, —ONR^{7c}R^{8c}, —NHC═(O)NHNH₂, —NHC═(O)NR^{7c}R^{8c}, —N(O)ₘ₃, —NR^{7c}R^{8c}, —C(O)R^{9c}, —C(O)—OR^{9c}, —C(O)NR^{7c}R^{8c}, —OR^{10c}, —NR^{7c}SO₂R^{10c}, —NR^{7c}C═(O)R^{9c}, —NR^{7c}C(O)—OR^{9c}, —NR^{7c}OR^{9c}, —OCX^{c}₃, —OCHX^{c}₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl.

R^{7c}, R^{8c}, R^{9c} and R^{10e} are independently hydrogen, halogen, —CF₃, —N₃, —CN, —OH, —NH₂, —COOH, —CONH₂, —NO₂, —SH, —SO₂Cl, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl, unsubstituted heteroalkyl, unsubstituted cycloalkyl, unsubstituted heterocycloalkyl, unsubstituted aryl, or unsubstituted heteroaryl. R^{7c} and R^{8c} substituents bonded to the same nitrogen atom may optionally be joined to form a substituted or unsubstituted heterocycloalkyl or substituted or unsubstituted heteroaryl.

In embodiments, R^{1C} is R²⁰-substituted or unsubstituted alkyl (e.g. C₁-C₈ alkyl, C₁-C₆ alkyl, or C₁-C₄ alkyl), R²⁰-substituted or unsubstituted heteroalkyl (e.g. 2 to 8 membered heteroalkyl, 2 to 6 membered heteroalkyl, or 2 to 4 membered heteroalkyl), R²⁰-substituted or unsubstituted cycloalkyl (e.g. C₃-C₈ cycloalkyl, C₄-C₈ cycloalkyl, or C₅-C₆ cycloalkyl), R²⁰-substituted or unsubstituted heterocycloalkyl (e.g. 3 to 8 membered heterocycloalkyl, 4 to 8 membered heterocycloalkyl, or 5 to 6 membered heterocycloalkyl), R²⁰-substituted or unsubstituted aryl (e.g. C₆-C₁₀ aryl or C₆ aryl), or R²⁰-substituted or unsubstituted heteroaryl (e.g. 5 to 10 membered heteroaryl, 5 to 9 membered heteroaryl, or 5 to 6 membered heteroaryl).

In some embodiments, R^{1D} is R²¹-substituted or unsubstituted alkyl (e.g. C₁-C₈ alkyl, C₁-C₆ alkyl, or C₁-C₄ alkyl), R²¹-substituted or unsubstituted heteroalkyl (e.g. 2 to 8 membered heteroalkyl, 2 to 6 membered heteroalkyl, or 2 to 4 membered heteroalkyl), R²¹-substituted or unsubstituted cycloalkyl (e.g. C₃-C₈ cycloalkyl, C₄-C₈ cycloalkyl, or C₅-C₆ cycloalkyl), R²¹-substituted or unsubstituted heterocycloalkyl (e.g. 3 to 8 membered heterocycloalkyl, 4 to 8 membered heterocycloalkyl, or 5 to 6 membered heterocycloalkyl), R²¹-substituted or unsubstituted aryl (e.g. C₆-C₁₀ aryl or C₆ aryl), or R²¹-substituted or unsubstituted heteroaryl (e.g. 5 to 10 membered heteroaryl, 5 to 9 membered heteroaryl, or 5 to 6 membered heteroaryl).

In embodiments, R^{2C} is R²²-substituted or unsubstituted alkyl (e.g. C₁-C₈ alkyl, C₁-C₆ alkyl, or C₁-C₄ alkyl), R²²-substituted or unsubstituted heteroalkyl (e.g. 2 to 8 membered heteroalkyl, 2 to 6 membered heteroalkyl, or 2 to 4 membered heteroalkyl), R²²-substituted or unsubstituted cycloalkyl (e.g. C₃-C₈ cycloalkyl, C₄-C₈ cycloalkyl, or C₅-C₆ cycloalkyl), R²²-substituted or unsubstituted heterocycloalkyl (e.g. 3 to 8 membered heterocycloalkyl, 4 to 8 membered heterocycloalkyl, or 5 to 6 membered heterocycloalkyl), R²²-substituted or unsubstituted aryl (e.g. C₆-C₁₀ aryl or C₆ aryl), or R²²-substituted or unsubstituted heteroaryl (e.g. 5 to 10 membered heteroaryl, 5 to 9 membered heteroaryl, or 5 to 6 membered heteroaryl).

In some embodiments, R^{2D} is R²³-substituted or unsubstituted alkyl (e.g. C₁-C₈ alkyl, C₁-C₆ alkyl, or C₁-C₄ alkyl), R²³-substituted or unsubstituted heteroalkyl (e.g. 2 to 8 membered heteroalkyl, 2 to 6 membered heteroalkyl, or 2 to 4 membered heteroalkyl), R²³-substituted or unsubstituted cycloalkyl (e.g. C₃-C₈ cycloalkyl, C₄-C₈ cycloalkyl, or C₅-C₆ cycloalkyl), R²³-substituted or unsubstituted heterocycloalkyl (e.g. 3 to 8 membered heterocycloalkyl, 4 to 8 membered heterocycloalkyl, or 5 to 6 membered heterocycloalkyl), R²³-substituted or unsubstituted aryl (e.g. C₆-C₁₀ aryl or C₆ aryl), or R²³-substituted or unsubstituted heteroaryl (e.g. 5 to 10 membered heteroaryl, 5 to 9 membered heteroaryl, or 5 to 6 membered heteroaryl).

In embodiments, R^{4C} is R²⁴-substituted or unsubstituted alkyl (e.g. C₁-C₈ alkyl, C₁-C₆ alkyl, or C₁-C₄ alkyl), R²⁴-substituted or unsubstituted heteroalkyl (e.g. 2 to 8 membered heteroalkyl, 2 to 6 membered heteroalkyl, or 2 to 4 membered heteroalkyl), R²⁴-substituted or unsubstituted cycloalkyl (e.g. C₃-C₈ cycloalkyl, C₄-C₈ cycloalkyl, or C₅-C₆ cycloalkyl), R²⁴-substituted or unsubstituted heterocycloalkyl (e.g. 3 to 8 membered heterocycloalkyl, 4 to 8 membered heterocycloalkyl, or 5 to 6 membered heterocycloalkyl), R²⁴-substituted or unsubstituted aryl (e.g. C₆-C₁₀ aryl or C₆ aryl), or R²⁴-substituted or unsubstituted heteroaryl (e.g. 5 to 10 membered heteroaryl, 5 to 9 membered heteroaryl, or 5 to 6 membered heteroaryl).

In some embodiments, R^{4D} is R²⁵-substituted or unsubstituted alkyl (e.g. C₁-C₈ alkyl, C₁-C₆ alkyl, or C₁-C₄ alkyl), R²⁵-substituted or unsubstituted heteroalkyl (e.g. 2 to 8 membered heteroalkyl, 2 to 6 membered heteroalkyl, or 2 to 4 membered heteroalkyl), R²⁵-substituted or unsubstituted cycloalkyl (e.g. C₃-C₈ cycloalkyl, C₄-C₈ cycloalkyl, or C₅-C₆ cycloalkyl), R²⁵-substituted or unsubstituted heterocycloalkyl (e.g. 3 to 8 membered heterocycloalkyl, 4 to 8 membered heterocycloalkyl, or 5 to 6 membered heterocycloalkyl), R²⁵-substituted or unsubstituted aryl (e.g. C₆-C₁₀ aryl or C₆ aryl), or R²⁵-substituted or unsubstituted heteroaryl (e.g. 5 to 10 membered heteroaryl, 5 to 9 membered heteroaryl, or 5 to 6 membered heteroaryl).

In embodiments, LG is an electron withdrawing group. The term “electron withdrawing group” refers, in the usual and customary sense, to a group which can reduce electron density in an attached functionality. In embodiments, LG is N₂⁺, —OR^{3A}₂², —OSO₂R^{3A}F, a perfluoroalkylsulfonate, a tosylate, a mesylate, a halogen; —OH₂⁺, —ONO₂, —OPO(OH)₂, —ONO₂, —S(R^{3B}R^{3C})⁺, —N(R^{3A}R^{3B}R^{C})⁺, —OCOR^{3A}, substituted or unsubstituted aryloxy or substituted or unsubstituted heteroaryloxy; and R^{3A}, R^{3B} and R^{3C} are independently hydrogen, halogen, —CF₃, —CCl₃, —CBr₃, —CI₃, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCCl₃, —OCBr₃, —OCI₃, —OCHF₂, —OCHC1₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3B} and R^{3C} substituents bonded to the same nitrogen atom may optionally be joined to form a substituted or unsubstituted heterocycloalkyl or substituted or unsubstituted heteroaryl.

In embodiments of the method, a solvent is present. In embodiments, the solvent is an organic solvent. Examples of organic solvents include, but are not limited to, hexanes, N,N-dimethylformamide, dimethylsulfoxide, toluene, benzene, xylenes, dichloromethane, chloroform, tetrahydrofuran, diethyl ether, dioxane, methyl tert-butyl ether, trimethylamine, pyridine, 1,3-dimethyl-2-imidazolidinone, N-Methylpyrrolidone, diisopropylamine, acetonitrile, acetone, ethyl acetate, iso-propyl acetate, ethanol, tert-butanol, methanol, phenol. In emodiments, the solvent is an inorganic solvent, such as water.

In embodiments of the method, a base is present. In embodiments, the base is an organic base. In embodiments, the base is trimethylamine or dicyclohexylmethylamine. In embodiments, the base is an inorganic base, such as potassium carbonate.

In embodiments, the method is conducted at a temperature from about 25° C. to 200° C.

In embodiments, the method is conducted at a temperature from about 40° C. to 150° C. In embodiments, the method is conducted at a temperature from about 50° C. to 140° C. In embodiments, the method is conducted at a temperature from about 50° C. to 130° C. In embodiments, the method is conducted at a temperature from about 50° C. to 120° C. In embodiments, the method is conducted at a temperature from about 50° C. to 110° C. In embodiments, the method is conducted at a temperature from about 50° C. to 100° C. In embodiments, the method is conducted at a temperature from about 50° C. to 90° C. In embodiments, the method is conducted at a temperature from about 50° C. to 80° C.

In embodiments, the method is conducted using electromagnetic radiation. In embodiments, the method is microwave-assisted. In embodiments, the method is conducted in microwave reactor. In embodiments, the method is conducted at a temperature from about 40° C. to 80° C.

In embodiments, the catalyst is Pd(OAc)₂, PdCl₂, PdCl₂(CH₃CN)₂, Pd₂(dba)₃Pd(PPh₃)₄ or Pd(dba)₂.

In embodiments, the ligand is (acetonitrile)[2-(dicyclohexylphosphino)3,6-dimethoxy-2′,4′,6′-triisopropyl-1,1′-biphenyl]gold(I) hexafluoroantimonate allyldiphenylphosphine, allyldiphenylphosphine oxide, (2-ammonioethyl)di-tert-butylphosphonium bis(tetrafluoroborate), (2-ammonioethyl)diisopropylphosphonium bis(tetrafluoroborate), (3-ammoniopropyl)diisopropylphosphonium bis(tetrafluoroborate), benzyldiphenylphosphine, 1-[2-[bis(tert-butyl)phosphino]phenyl]-3,5-diphenyl-1h-pyrazole, bis[2-(diadamantylphosphino)ethyl]amine, bis(5h-dibenzo[a,d]cyclohepten-5-yl)phenylphosphine, bis(5h-dibenzo[a,d]cyclohepten-5-yl)phenylphosphine, 2-[bis(3,5-di-tert-butyl-4-methoxyphenyl)phosphino]benzaldehyde, 2,6-bis(di-tert-butylphosphinomethyl)pyridine, bis(dicyclohexylphosphinophenyl) ether, bis(diethylamino)phenylphosphine, 1,3-bis-(2,6-diisopropylphenyl)-[1,3,2]diazaphospholidine 2-oxide, bis(dimethylamino)chlorophosphine, 2-[bis(3,5-dimethylphenyl)phosphino]benzaldehyde, 2,2′-bis(diphenylphosphino)-1,1′-biphenyl, bis(4-fluorophenyl)phenylphosphine oxide, bis[4-(3,3,4,4,5,5,5-heptafluoro-2,2-bis(trifluoromethyl)pentyl)phenyl]phenylphosphine, 1,1′-bis(phenylphosphinidene)ferrocene, bis(triphenylphosphine)palladium(ii) dichloride, (2-bromophenyl)dicyclohexylphosphine, (2-bromophenyl)diphenylphosphine, tert-butyldicyclohexylphosphine, tert-butyldicyclohexylphosphonium tetrafluoroborate, tert-butyldiisopropylphosphine, tert-butyldiphenylphosphine, di-tert-butyl(2,2-diphenyl-1-methyl-1-cyclopropyl)phosphine, 2-chloro-1,3-bis(2,6-diisopropylphenyl)-1,3,2-diazaphospholidine, 2-dicyclohexylphosphino-2′,6′-bis(n,n-dimethylamino)biphenyl, 1-(dicyclohexylphosphino)-2,2-diphenyl-1-methylcyclopropane, dicyclohexyl(2,2-diphenyl-1-methyl-1-cyclopropyl)phosphine, cyclohexyldiphenylphosphine, 2-(dicyclohexylphosphino)-1,1-diphenyl-1-propene, dicyclohexyl(1-methyl-2,2-diphenylvinyl)phosphine, di(1-adamantyl)-2-dimethylaminophenylphosphine, di-1-adamantylphosphine, di(1-adamantyl)-(2-triisopropylsiloxyphenyl)phosphine, (5h-dibenzo[a,d]cyclohepten-5-yl)diphenylphosphine, (R)-(−)-1-[(S)-2-(di(3,5-bis-trifluoromethylphenylphosphino)ferrocenyl]ethyldicyclohexylphosphine, (R)-(−)-1-[(S)-2-(di(3,5-bis-trifluoromethylphenyl)phosphino)ferrocenyl]ethyldi(3,5-dimethylphenyl)phosphine, p,p-dichloroferrocenylphosphine, (R)-(−)-n,n-dimethyl-1-[(S)-2-(diphenylphosphino)ferrocenyl]ethylamine or 1,2,3,4,5-pentaphenyl-1′-(di-tert-butylphosphino)ferrocene.

In embodiments, the method further includes a co-catalyst or additive. In embodiments, the co-catalyst or additive is Ag₂CO₃, LiCl, LiOAc, NaOAc, KF, Pd—Cu-exchanged montmorillonite K10 clay or AlCl₃.

In embodiments, the method is a cascade reaction or one-pot procedure.

In embodiments, the ligand is a phosphinoferrocene ligand. In embodiments, the ligand is 1,2,3,4,5-pentaphenyl-1′-(di-tert-butylphosphino)ferrocene. In embodiments, the catalyst is Pd₂(dba)₃.

In embodiments, the compound of Formula I is a 3-substituted-6-alkenyl-1,2,4,5-tetrazine.

In embodiments, X is iodine.

In embodiments, LG is —OMs.

In embodiments, L¹ and L² are independently substituted or unsubstituted C₁-C₆ alkylene.

In embodiments, the method further includes synthesizing a compound of Formula IV:

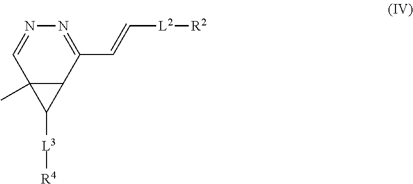

by reacting the compound of Formula I with a compound of Formula V:

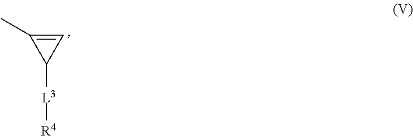

In compounds of Formula V, L³ is a bond or covalent linker; and R⁴ is a biomolecule, a dye or fluorophore.

L³ is independently a bond, —NR^{4C}—, —O—, —S—, —C(O)—, —S(O)—, —S(O)₂—, —C(O)—, —C(O)N(R^{4C})—, —N(R^{4C})S(O)—, —N(R^{4C})S(O)₂—, —C(O)OR^{4C}, —CH(R^{4A})N(C(O)OR^{4C})—, —CH(R^{4A})N(C(O)R^{4C})—, —CH(R^{4A})N(SO₂R^{4C})—, —CH(R^{4A})N(R^{4C})—, —CH(R^{4A})C(O)N(R^{4C})—, —CH(R^{4A})N(R^{4C})C(O)—, —CH(R^{4A})N(R^{4C})S(O)—, —CH(R^{4A})N(R^{4C})S(O)₂—, —N(R^{4C})C(O)N(R^{4D})—, substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. In embodiments, L³ is a bond or linker.

In embodiments, L³ is substituted or unsubstituted alkylene, substituted or unsubstituted heteroalkylene, substituted or unsubstituted cycloalkylene, substituted or unsubstituted heterocycloalkylene, substituted or unsubstituted arylene or substituted or unsubstituted heteroarylene. In embodiments, L³ is R¹³-substituted or unsubstituted alkylene (e.g. C₁-C₈ alkylene, C₁-C₆ alkylene, or C₁-C₄ alkylene), R¹³-substituted or unsubstituted heteroalkylene (e.g. 2 to 8 membered heteroalkylene, 2 to 6 membered heteroalkylene, or 2 to 4 membered heteroalkylene), R¹³-substituted or unsubstituted cycloalkylene (e.g. C₃-C₈ cycloalkylene, C₄-C₈ cycloalkylene, or C₅-C₆ cycloalkylene), R¹³-substituted or unsubstituted heterocycloalkylene (e.g. 3 to 8 membered heterocycloalkylene, 4 to 8 membered heterocycloalkylene, or 5 to 6 membered heterocycloalkylene), R¹³-substituted or unsubstituted arylene (e.g. C₆-C₁₀ arylene or C₆ arylene), or R¹³-substituted or unsubstituted heteroarylene (e.g. 5 to 10 membered heteroarylene, 5 to 9 membered heteroarylene, or 5 to 6 membered heteroarylene). R¹³ is oxo, halogen, —F, —Cl, —Br, —I, —CF₃, —CCl₃, —N₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. In embodiments, R¹³ is substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl. In embodiments, R¹³ is oxo, halogen, —F, —Cl, —Br, —I, —CF₃, —CCl₃, —N₃, —CN, —OH, —NH₂, —COOH, —CONH₂, —NO₂, —SH, —SO₃H, —SO₄H, —SO₂NH₂, —NHNH₂, —ONH₂, —NHC═(O)NHNH₂, —NHC═(O) NH₂, —NHSO₂H, —NHC═(O)H, —NHC(O)—OH, —NHOH, —OCF₃, —OCHF₂, unsubstituted alkyl (e.g. C₁-C₈ alkyl, C₁-C₆ alkyl, or C₁-C₄ alkyl), unsubstituted heteroalkyl (e.g. 2 to 8 membered heteroalkyl, 2 to 6 membered heteroalkyl, or 2 to 4 membered heteroalkyl), unsubstituted cycloalkyl (e.g. C₃-C₈ cycloalkyl, C₄-C₈ cycloalkyl, or C₅-C₆ cycloalkyl), unsubstituted heterocycloalkyl (e.g. 3 to 8 membered heterocycloalkyl, 4 to 8 membered heterocycloalkyl, or 5 to 6 membered heterocycloalkyl), unsubstituted aryl (e.g. C₆-C₁₀ aryl or C₆ aryl), or unsubstituted heteroaryl (e.g. 5 to 10 membered heteroaryl, 5 to 9 membered heteroaryl, or 5 to 6 membered heteroaryl).

In embodiments, L³ is —O—. In some embodiments, L³ is —S—. In some embodiments, L³ is —C(O)—. In some embodiments, L³ is —S(O)—. In some embodiments, L³ is —S(O)₂—. In embodiments, L³ is —NR^{4C}—. In embodiments, L³ is independently —CR^{4A}R^{4B}—. In embodiments, L³ is independently

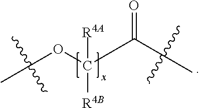

In embodiments, L³ is independently

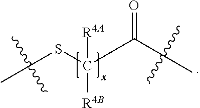

In embodiments, L³ is independently

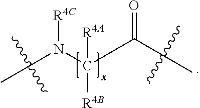

In embodiments, L³ is a bond. R⁴ is a biomolecule, a dye or fluorophore. L² and R² are as described herein, including embodiments.

The symbols m1, m3, v1, and v3 are independently an integer from 1 to 2. The symbols n1 and n3 are independently an integer from 0 to 4. The symbols X^{a} and X^{c} are independently —Cl, —Br, —I, or —F. The symbol v1 is independently 1 or 2. In some embodiments, v1 is 1. In some embodiments, v1 is 2. The symbol m1 is independently an integer from 1 to 2. In some embodiments, m1 is 1. In some embodiments, m1 is 2. The symbol n1 is independently an integer from 0 to 4. In some embodiments, n1 is 0. In some embodiments, n1 is 1. In some embodiments, n1 is 2. In some embodiments, n1 is 3. In some embodiments, n1 is 4. X^{a} is independently —Cl, —Br, —I, or —F. In some embodiments, X^{a} is —Cl. In some embodiments, X^{a} is —Br. In some embodiments, X^{a} is —I. In some embodiments, X^{a} is —F. The symbol v3 is independently 1 or 2. In some embodiments, v3 is 1. In some embodiments, v3 is 2. The symbol m3 is independently an integer from 1 to 2. In some embodiments, m3 is 1. In some embodiments, m3 is 2. The symbol n3 is independently an integer from 0 to 4. In some embodiments, n3 is 0. In some embodiments, n3 is 1. In some embodiments, n3 is 2. In some embodiments, n3 is 3. In some embodiments, n3 is 4. X^{c} is independently —Cl, —Br, —I, or —F. In some embodiments, X^{c} is —Cl. In some embodiments, X^{c} is —Br. In some embodiments, X^{c} is —I. In some embodiments, X^{c} is —F. The symbols w, x, y and z are independently an integer from 0 to 10. In some embodiments, w is 0. In some embodiments, w is 1. In some embodiments, w is 2. In some embodiments, w is 3. In some embodiments, w is 4. In some embodiments, w is 5. In some embodiments, w is 6. In some embodiments, w is 7. In some embodiments, w is 8. In some embodiments, w is 9. In some embodiments, w is 10. In some embodiments, x is 0. In some embodiments, x is 1. In some embodiments, x is 2. In some embodiments, x is 3. In some embodiments, x is 4. In some embodiments, x is 5. In some embodiments, x is 6. In some embodiments, x is 7. In some embodiments, x is 8. In some embodiments, x is 9. In some embodiments, x is 10. In some embodiments, y is 0. In some embodiments, y is 1. In some embodiments, y is 2. In some embodiments, y is 3. In some embodiments, y is 4. In some embodiments, y is 5. In some embodiments, y is 6. In some embodiments, y is 7. In some embodiments, y is 8. In some embodiments, y is 9. In some embodiments, y is 10. In some embodiments, z is 0. In some embodiments, z is 1. In some embodiments, z is 2. In some embodiments, z is 3. In some embodiments, z is 4. In some embodiments, z is 5. In some embodiments, z is 6. In some embodiments, z is 7. In some embodiments, z is 8. In some embodiments, z is 9. In some embodiments, z is 10.

In embodiments, the dye is a xanthene dye or a boron-dipyrromethene dye.

In another aspect, there is provided a method of detecting a biomolecule of interest. The method includes:

(i) contacting the compound of Formula I as disclosed herein, wherein R² is a fluorophore, with a compound of Formula V:

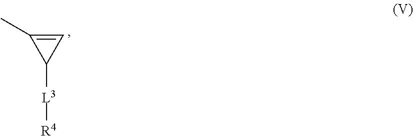

and

(ii) detecting the level of fluorescence, wherein an increase in fluorescence compared to a control is indicative of the presence of the biomolecule. In compound of Formula V, L³ is a covalent bond or linker. R⁴ is the biomolecule. The term “biomolecule of interest” refers to a biomolecule (e.g., lipid, protein, peptide, nucleic acid, carbohydrate and the like) for which a qualitative and/or quantitative level is desired.

In embodiments, the biomolecule is in a cell. In embodiments, the cell is a live cell. In embodiments, the cell is a human cell.

In embodiments, the biomolecule is a lipid, nucleic acid, protein or carbohydrate. In embodiments, the biomolecule is a phospholipid or antibody.

### II. Compounds

In another aspect, there is provided a compound of Formula I:

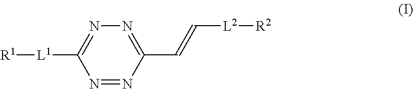

In another aspect, there is provided a compound of Formula IIa:

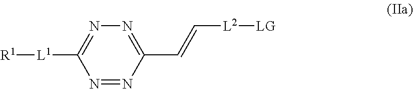

L¹,L²,R¹, R², LG and X are as described herein including embodiments thereof.

In embodiments, the compound is:

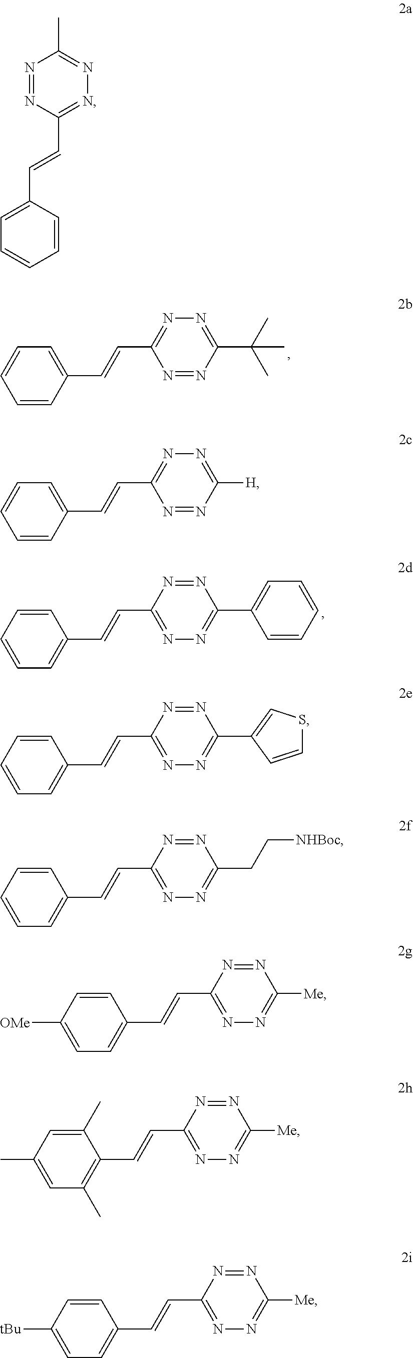

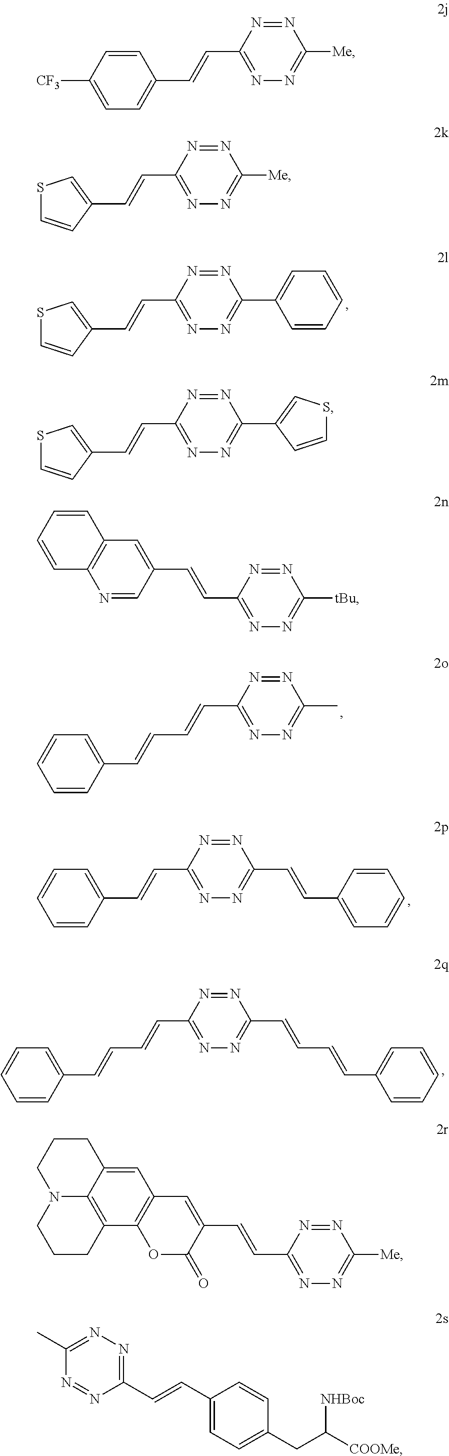

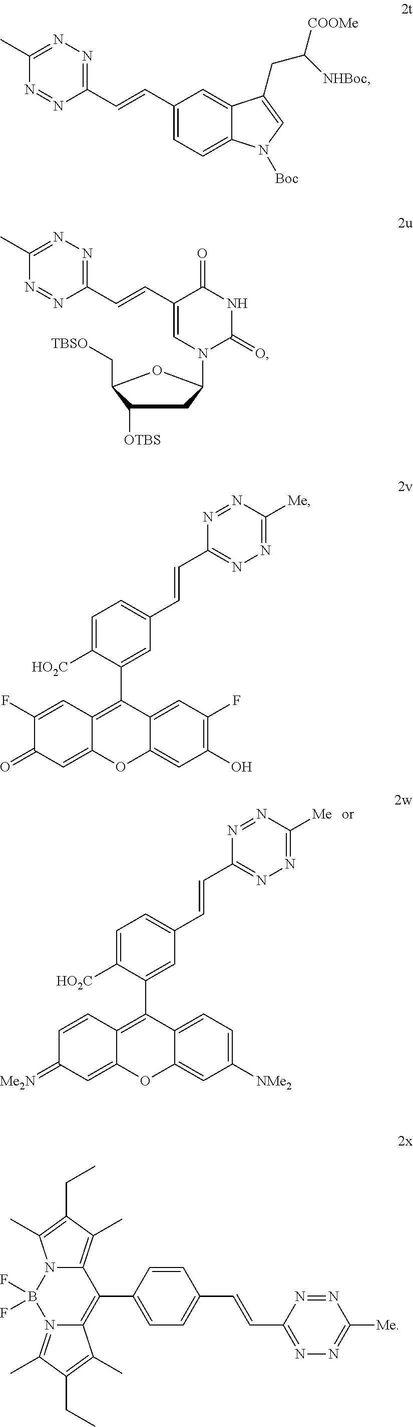

In another aspect, there is provided a compound of Formula IV:

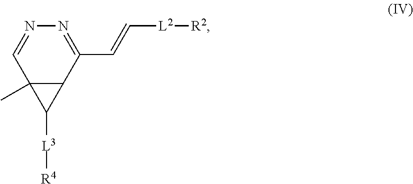

L²,L³,R² and R⁴ are as described herein including embodiments thereof.

### III. Examples

The following examples are for the purpose of illustrating particular embodiments of the invention described herein and are not intended to limit the scope of the invention.

We envisioned, inter alia, developing a simple tetrazine building block that was stable, could be easily synthesized, and readily installed onto complex substrates, including commonly used fluorescent probes, under mild conditions. In previous studies, s-dichlorotetrazine and s-dithiomethyltetrazine were used to prepare numerous functional s-tetrazines via nucleophilic displacement.[1a,21] Related tetrazines undergo S_{N}Ar reactions with carbanions and limited cross-coupling reactions; however, the reactions take place only if the tetrazine is deactivated by one donating substituent (alkylamino, alkoxy, or alkylthio), and the desired products are obtained in moderate yield, greatly restricting the possible tetrazine derivatives and potential applications.[9] For instance, although 1,2,4,5-tetrazines have been used in bioorthogonal reactions, owing to their high reactivity in inverse-electron demand Diels-Alder cycloadditions, mono(bis)-alkylamino (alkoxy, alkylthio) substituted tetrazine derivatives are not expected to be rapidly reacting due to the electronic effects of the electron-donating groups.[10]

Provided herein, inter alia, is an in situ synthesis of (E)-3-substituted-6-alkenyl-1,2,4,5-tetrazine derivatives via an elimination-Heck cascade reaction. This method enables convenient introduction of 3-substituted-6-alkenyl-1,2,4,5-tetrazine moieties onto a diverse array of functional molecules. These include unnatural nucleotides and amino acids that are relevant to bioorthogonal chemistry applications. The technique can also be used to readily prepare unique π-conjugated 1,2,4,5-tetrazine derivatives that are either difficult or not possible to prepare using alternative synthetic strategies, facilitating the future use of t-conjugated tetrazines as electron-deficient components in molecular electronics, photovoltaics, and non-linear optics.[1a,6] Finally, we demonstrate the ability to synthesize a diverse set of tetrazine fluorogenic probes, both from xanthene and BODIPY® precursors. The term “BODIPY®,” “BODIPY® dye” and the like refer, in the usual and customary sense, to a class of fluorescent dyes including a boron-dipyrromethene functionality. Due to conjugation between the alkenyl tetrazine and the fluorescent core, these dyes show excellent fluorogenic properties after reaction with dienophiles, with turn-on ratios up to 400-fold. We demonstrate their suitability for live-cell imaging applications by detecting dienophile modified cell surface markers.

We developed, inter alia, a series of novel tetrazine building blocks, which can smoothly react with aryl halides via a mild and high-yielding in situ elimination-Heck cascade reaction leading to the formation of (E)-3-substituted-6-alkenyl-1,2,4,5-tetrazines. This method enables convenient preparation of highly conjugated 1,2,4,5-tetrazines, including previously unreported buta-1,3-diene substituted 1,2,4,5-tetrazines. Moreover, this methodology provides a new strategy to prepare highly quenched fluorogenic tetrazines, including derivatives of popular dyes (e.g., xanthene and BODIPY® dyes) suitable for live-cell imaging applications. This methodology can greatly facilitate the study of 1,2,4,5-tetrazines, advancing their further application in chemical biology, material science, electrochemistry, photovoltaics, nonlinear optics, and particularly live-cell imaging.

We hypothesized that 3-substituted-6-vinyl tetrazines could be versatile tetrazine building blocks readily appended onto a diverse array of molecules using the Heck coupling reaction. However, there have been very few reports of alkenyl-modified tetrazines, and unsymmetric vinyl tetrazines are unknown, likely due to the previous difficulty in synthesizing unsymmetric tetrazines as well as the potential volatility of simple vinyl-tetrazines. [11] As mentioned, we recently disclosed a straightforward route to unsymmetric tetrazines, and using this technique we synthesized 3-hydroxyethyl-6-methyl-tetrazine in one-pot fashion from commercially available starting materials.[7] Mesylation of 3-hydroxyethyl-6-methyl-tetrazine, to form 1a, followed by elimination led to 3-methyl-6-vinyl tetrazine. As expected, the resulting vinyl tetrazine was very volatile and not convenient to isolate. In contrast, tetrazine 1a is a stable and easily handled pink powder which can be stored at −20° C. for several months without noticeable decomposition. We therefore turned to precursor 1a as a substitute of 3-methyl-6-vinyl tetrazine and explored an in situ elimination-Heck cascade reaction.[12]

TABLE 1
Optimization of the reaction conditions.^{[a]}

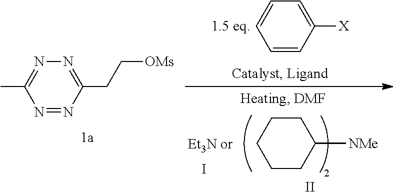

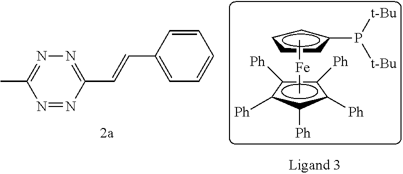

| | | | | Yield |
|---|---|---|---|---|
| Entry | Cat., Ligand | Heat, Time | Base | (%)^{[d]} |
| 1^{[b]} | 10% Pd(PPh₃)₄ | 80° C., 90 min | I | 0 |
| 2^{[b]} | 10% Pd(PPh₃)₄ | 50° C., MW 30 min | II | 55 |
| 3^{[b]} | 10% Pd₂(dba)₃ | 50° C., MW 30 min | II | 80 |
| | 40% P(o-Tol)₃ | | | |
| 4^{[c]} | 3% Pd₂(dba)₃, | 60° C., MW 40 min | II | 58 |
| | 12% (t-Bu)₃P⁺BF₄⁻ | | | |
| 5^{[b], [c]} | 3% Pd₂(dba)₃ | 50° C., MW 30 min | II | 99 |
| | 12% ligand 3 | | | |

^{[a]}All reactions were carried out on a 0.02 mmol scale in 1.5 mL DMF. Ms—Mesyl group. MW—microwave.
^{[b]}Iodobenzene as starting material.
^{[c]}Bromobenzene as starting material.
^{[d]}Isolated yield based on 1a, no (Z)-3-methyl-6-styryl-s-tetrazine was observed.

We initially screened the reaction conditions for the Heck cascade reaction of 1a with iodobenzene using common catalysts, ligands, bases, and solvents. However, using 1.5 equivalents (eq) iodobenzene, 10% Pd(PPh₃)₄/Et₃N/DMF and heating to 80° C. for 90 min, led to no observable product (Table 1, Entry 1). This result is in agreement with past difficulties in using standard Heck coupling conditions with tetrazines.[91] In recent years, microwave irradiation has been used in cross-coupling reactions and we therefore decided to explore the use of microwave activation for the Heck cascade reaction. [13] After a screening of catalysts, ligands[14] and bases (Table 1, Entry 2-5), we found use of Ligand 3 at 3% loading enabled the isolation of 2a in nearly quantitative yield from both iodobenzene and bromobenzene (Table 1, Entry 5).

TABLE 2
Substrate Scope [a], [b]

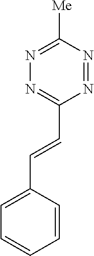 [SMILES: Cc1nnc(/C=C/c2ccccc2)nn1]

2a, 1.5 eq
30 min, 99%

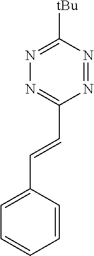 [SMILES: CC(C)(C)c1nnc(/C=C/c2ccccc2)nn1]

2b, 1.5 eq
30 min, 97%

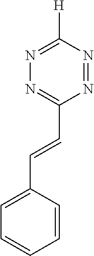 [SMILES: C(=C\c1nncnn1)/c1ccccc1]

2c, 1.5 eq
30 min, 83%^{c}

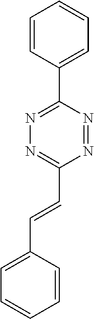 [SMILES: C(=C\c1nnc(-c2ccccc2)nn1)/c1ccccc1]

2d, 1.5 eq
35 min, 99%

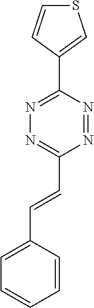 [SMILES: C(=C\c1nnc(-c2ccsc2)nn1)/c1ccccc1]

2e, 1.5 eq
35 min, 82%

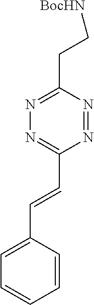 [SMILES: CC(C)(C)OC(=O)NCCc1nnc(/C=C/c2ccccc2)nn1]

2f, 1.5 eq
45 min, 91%

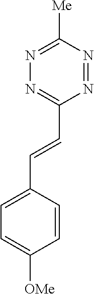 [SMILES: COc1ccc(/C=C/c2nnc(C)nn2)cc1]

2g, 1.2 eq
35 min, 93%

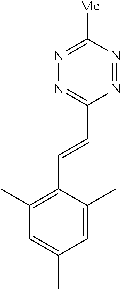

2h, 1.2 eq
40 min, 97%

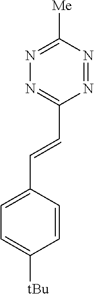 [SMILES: Cc1nnc(/C=C/c2ccc(C(C)(C)C)cc2)nn1]

2i, 1.5 eq
30 min, 96%

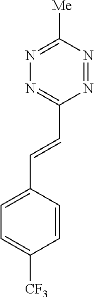 [SMILES: Cc1nnc(/C=C/c2ccc(C(F)(F)F)cc2)nn1]

2j, 1.2 eq
40 min, 86%

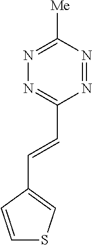 [SMILES: Cc1nnc(/C=C/c2ccsc2)nn1]

2k, 1.4 eq
40 min, 86%

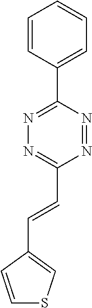 [SMILES: C(=C\c1nnc(-c2ccccc2)nn1)/c1ccsc1]

2l, 1.4 eq
40 min, 82%

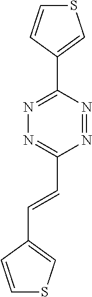 [SMILES: C(=C\c1nnc(-c2ccsc2)nn1)/c1ccsc1]

2m, 1.4 eq
40 min, 58%

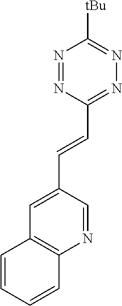 [SMILES: CC(C)(C)c1nnc(/C=C/c2cnc3ccccc3c2)nn1]

2n, 1.1 eq
40 min, 79%

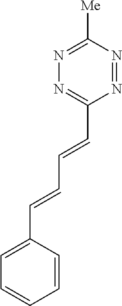 [SMILES: Cc1nnc(/C=C/C=C/c2ccccc2)nn1]

2o, 1.1 eq
35 min, 48%^{c, g}

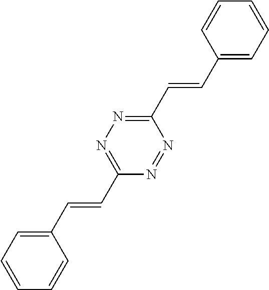 [SMILES: C(=C\c1nnc(/C=C/c2ccccc2)nn1)/c1ccccc1]

2p, 3.0 eq
30 min, 89%^{d, i}

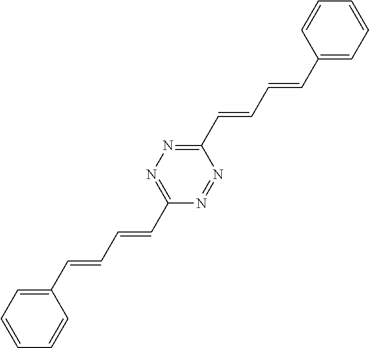 [SMILES: C(/C=C/c1nnc(/C=C/C=C/c2ccccc2)nn1)=C\c1ccccc1]

2q, 2.2 eq
35 min, 58%^{e, g, i}

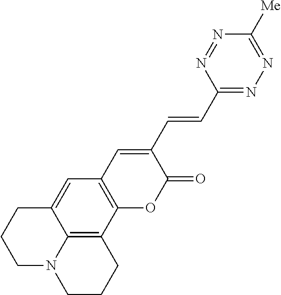 [SMILES: Cc1nnc(/C=C/c2cc3cc4c5c(c3oc2=O)CCCN5CCC4)nn1]

2r, 1.0 eq
40 min, 83%^{c, f}

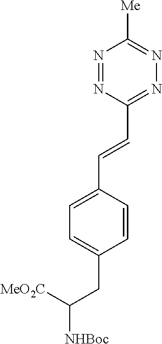 [SMILES: COC(=O)C(Cc1ccc(/C=C/c2nnc(C)nn2)cc1)NC(=O)OC(C)(C)C]

2s, 1.1 eq
60 min, 76%^{c}

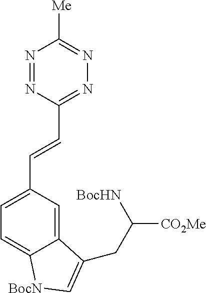 [SMILES: COC(=O)C(Cc1cn(C(=O)OC(C)(C)C)c2ccc(/C=C/c3nnc(C)nn3)cc12)NC(=O)OC(C)(C)C]

2t, 1.1 eq
30 min, 55%^{c, h}

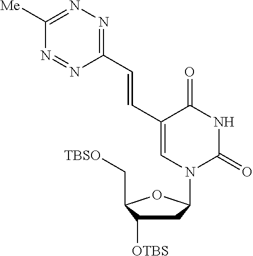

2u, 1.1 eq
60 min, 47%^{c, f}

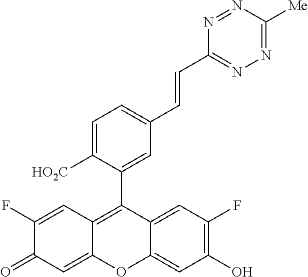 [SMILES: Cc1nnc(/C=C/c2ccc(C(=O)O)c(-c3c4cc(F)c(=O)cc-4oc4cc(O)c(F)cc34)c2)nn1]

2v, 1.1 eq
40 min, 58%^{e, j}

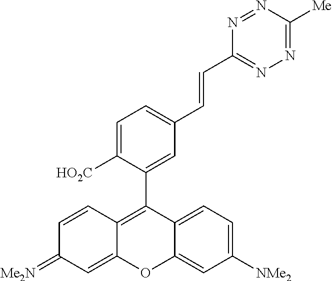

2w, 1.1 eq
40 min, 53%^{e, j}

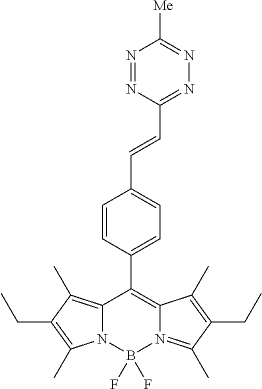

2x, 1.0 eq
35 min, 83%^{c, f}
[a] Conducted on 0.02 mmol scale. Reaction time and equivalents of bromide compounds shown under each product, 3 mol % Pd₂(dba)₃, 12 mol % ligand 3, 3 eq. Cy₂NMe, microwave 50° C.
[b] Isolated yield.
^{[c]}5 mol % Pd₂(dba)₃, 20 mol % ligand 3.
^{[d]}6 mol % Pd₂(dba)₃, 24 mol % ligand 3.
^{[e]}10 mol % Pd₂(dba)₃, 40 mol % ligand 3.
^{[f]}Iodide as starting material.
^{[g]}microwave 55° C.
^{[h]}microwave 60° C.
^{[i]}6 eq. Cy₂NMe.
^{[j]}4 eq. Cy₂NMe.

With these optimized conditions in hand, we surveyed the substrate scope of the in situ elimination-Heck reaction (Table 2). Substitutions at the 3-position of the alkenyl-tetrazines could be introduced by the synthesis and use of alternative vinyl tetrazine precursors. In this fashion, t-butyl, unsubstituted, phenyl, heterocyclic, and protected-amine alkenyl-tetrazine coupling products could be obtained in high yields (2b-2f). Diversified phenyl bromides possessing sterically bulky, electron-donating, electron-withdrawing and heterocyclic substituents were also tolerated by the reaction conditions and gave the corresponding alkenyl tetrazines in excellent to good yields (2g-2n). Interestingly, reduction of the double bond of alkenyl tetrazines such as 2a through hydrogenation was possible, providing a novel route to unsymmetric alkyl substituted 1,2,4,5-tetrazines.

Despite significant interest in incorporating electron deficient tetrazine heterocycles in conjugated bridges, a roadblock has been the lack of accessible methods to synthesize π-conjugated tetrazines, particularly alkenyl substituted tetrazines, of which there are very few reported[11,15] and longer conjugated buta-1,3-diene substituted tetrazines which, to our knowledge, are unknown in the literature. Remarkably, using the Heck cascade reaction, we were also able to readily synthesize conjugated mono-phenylbutadiene, bistyryl and biphenylbutadiene substituted s-tetrazines (2o -2q) in moderate to good yield.

We next examined the installation of bioorthogonal tetrazine handles on several biologically relevant and functionally complex molecules such as coumarin, deoxyribose, and amino acid derivatives.[1c,3,4,16] Under the modified Heck reaction conditions, these substrates smoothly reacted with 1a and delivered 2r-2u in 47%-83% yield. Deprotection of 2s-2u, gave unnatural tetrazine modified deoxyuridine 12, DL-phenylalanine 13 and DL-tryptophan 14. We evaluated the stability of (E)-3-substituted-6-styryl-s-tetrazines 2a-c in aqueous solutions and in the presence of biologically relevant nucleophiles such as thiols. Additionally, we measured the reaction kinetics between alkenyl tetrazines 2a-c and a highly strained trans-cyclooctene (TCO) dienophile. Stability and reactivity trends were consistent with past observations.[17]

A major application of tetrazine ligations has been the live-cell imaging of dienophile tagged small molecules, including proteins, lipids, sugars, and drug analogs.[2b, 3a, 3b, 16a, 16b, 18] These applications are aided by the existence of fluorogenic tetrazines, which consist of popular fluorophores for cellular imaging such as xanthene and BODIPY® dyes quenched through energy transfer by a tetrazine handle.[19] It is observed that when tetrazines are appended through aliphatic linkers, fluorescence increases after ligation are typically 10-fold and background signal from unreactive fluorophore limits the sensitivity of detection. [4] Fluorogenic tetrazine probes possessing fluorescence intensity increases greater than 100 fold after ligation would enable far more sensitive detection of dienophile targets. Recently, it was demonstrated that tetrazines could be appended directly onto BODIPY® fluorophores resulting in highly fluorogenic probes that were quenched by through bond energy transfer (TBET). [5] Unfortunately, the harsh conditions required for heterocycle synthesis resulted in low yields and the technique was only demonstrated for a green emitting BODIPY® dye. Conventional BODIPY® probes have several drawbacks, including a small Stokes shift[20] and poor aqueous solubility.[21] In contrast, xanthene dyes, such as fluorescein and rhodamine derivatives, are arguably the most popular class of fluorescent probes for cellular imaging and are typically highly soluble in aqueous solutions. However, to date, highly quenched tetrazine xanthene dyes have not been demonstrated.

We applied our method to synthesize tetrazine-conjugated xanthene dyes such as 2′,7′-Difluorofluorescein (Oregon-Green)-tetrazine derivative 2v and Tetramethylrhodamine-tetrazine 2w in 58% and 53% yield respectively. Additionally, BODIPY®-tetrazine dye 2x could be synthesized in 83% yield. All fluorophore-alkenyl-tetrazines were highly quenched, but became strongly emissive upon reaction with dienophiles (Scheme 1). Oregon-Green-tetrazine derivative 2v showed the highest turn-on with 135-fold and 400-fold increases in fluorescence intensity after reaction with a cyclopropene[23] and a TCO respectively (Scheme 1 step a)). The alkenyl-tetrazine fluorophores were stable when stored at −80° C. for 1 month and remained stable in solution over 24 hours. Rhodamine 2w also showed significant turn-on (up to 76-fold), with maximum emission in the red (569-573 nm) after reaction with dienophiles. This indicates that appropriately conjugated tetrazines can significantly quench both green and red emitting dyes, opening up the possibility of two-color imaging using highly fluorogenic tetrazine probes.

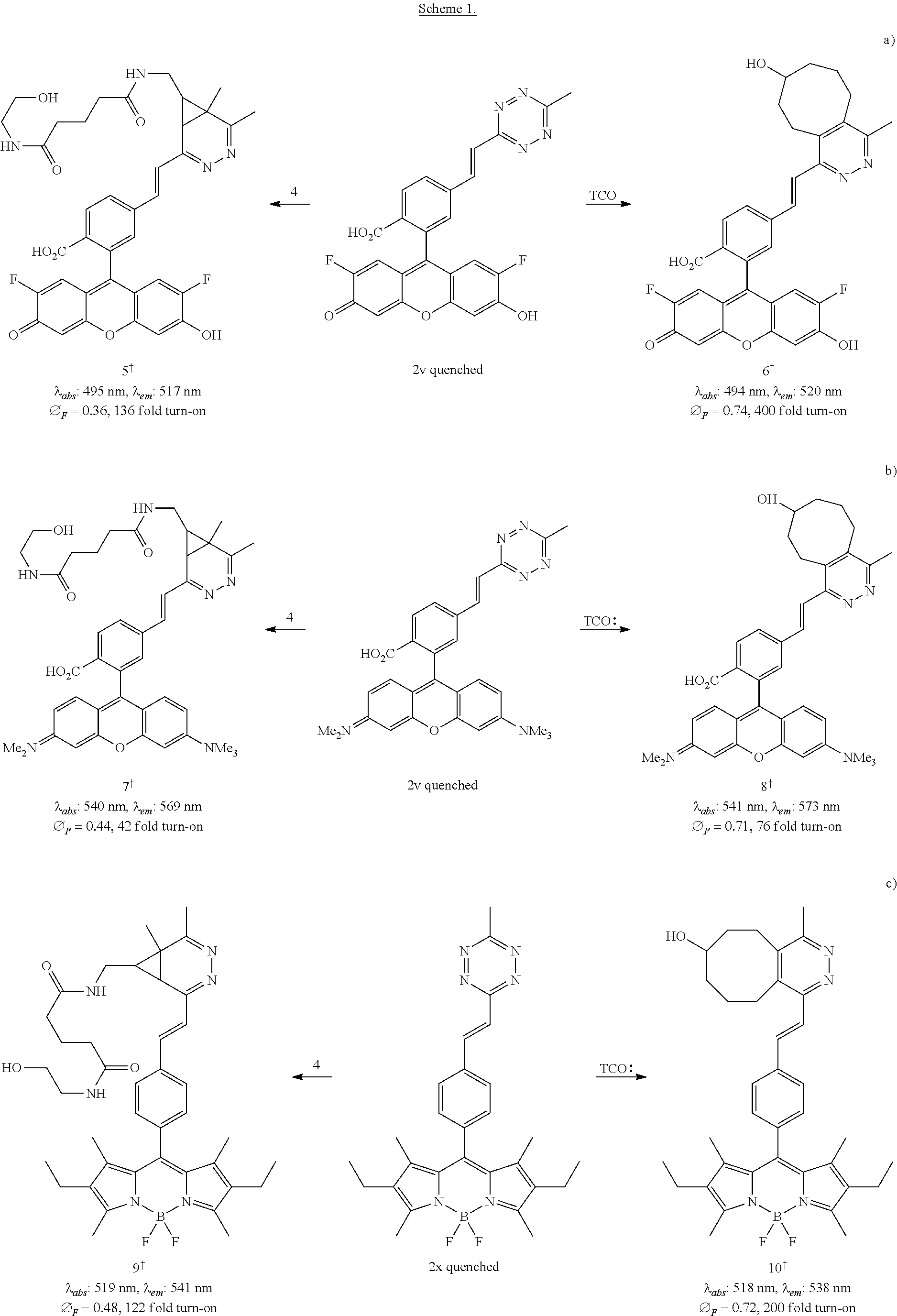

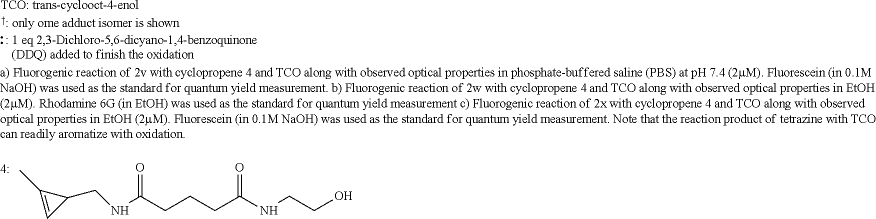

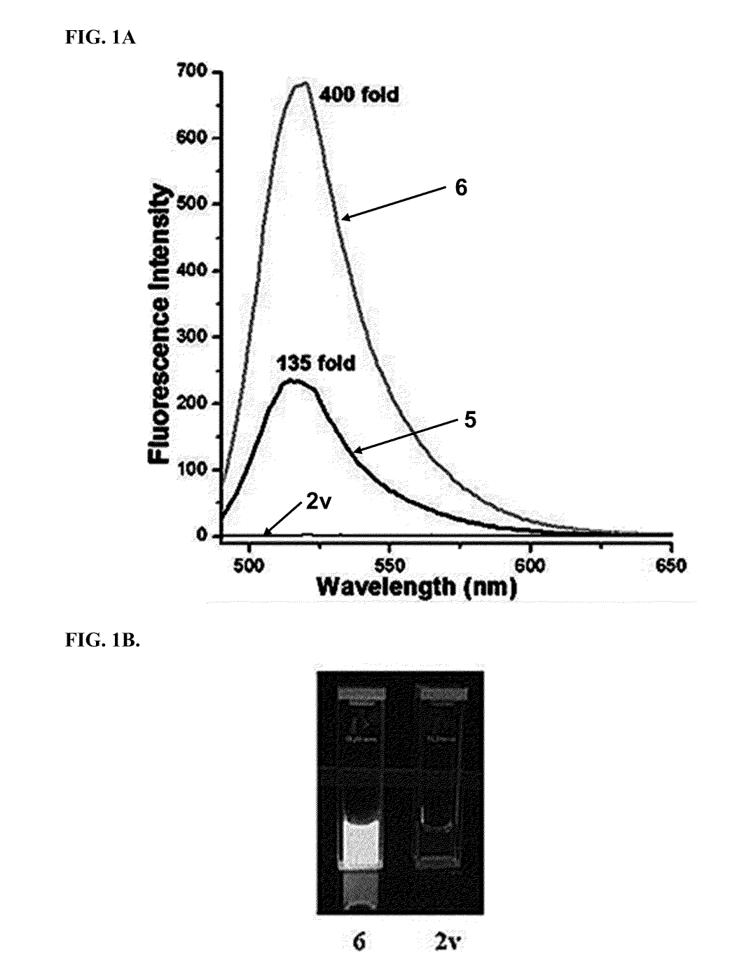

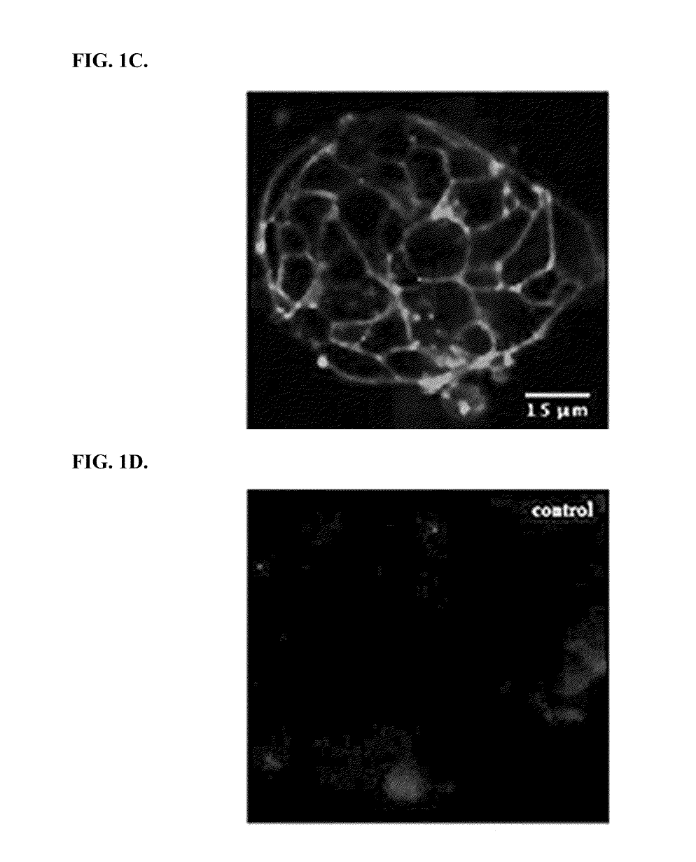

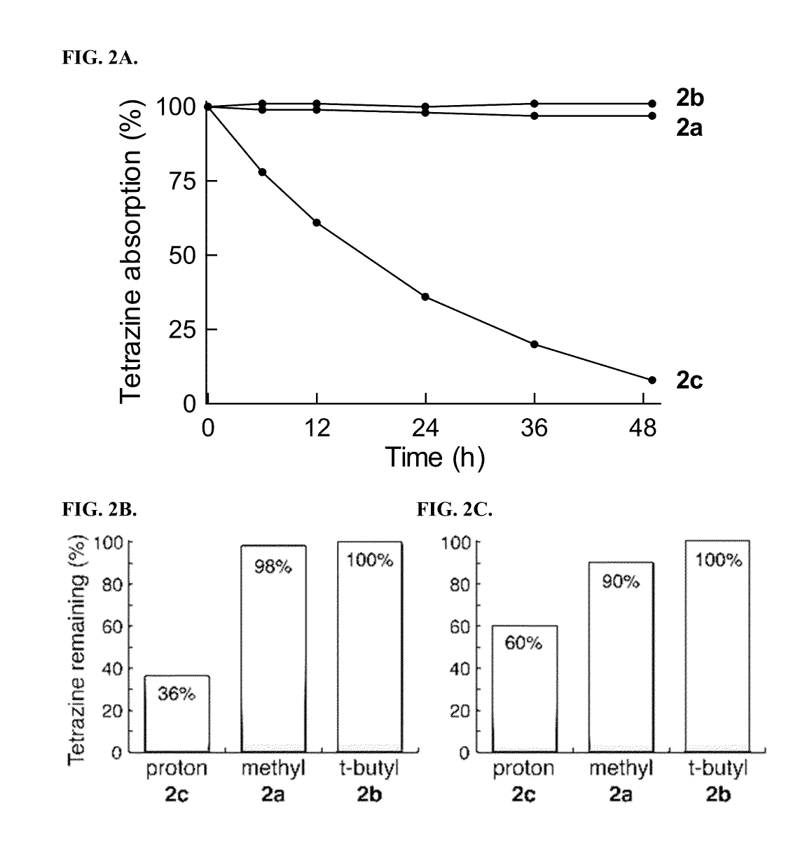

To demonstrate the suitability of quenched alkenyl-tetrazine probes for live-cell imaging, A33 antigens on live LS174T human colon carcinoma cells were pretargeted with TCO monoclonal antibodies. [24] Exposure to 2v followed by confocal imaging readily revealed the location of targeted dienophiles (FIG. 1C), while cells lacking targeted dienophile showed negligible background staining, a benefit of the highly fluorogenic nature of 2v.

### General Methods

All reagents were purchased from Sigma-Aldrich and used without further purification unless otherwise noted. The TLC plates used for purification were purchased from Agela Technologies (silica 200×200 mm, PH=5, MF=254, glass back). Other chromatographic purifications were conducted using 40-63 μm silica gel. The microwave reaction was done using a CEM microwave reactor. All mixtures of solvents are given in v/v ratio. ¹H and ¹³C NMR spectroscopy was performed on a Varian NMR at 400 (¹H) or 100 (¹³C) MHz and a Jeol NMR at 500 (¹H) or 125 (¹³C) MHz. All ¹³C NMR spectra were proton decoupled. Fluorescence measurements were done using a Perkin Elmer LD-45 spectrophotometer equipped with a single cuvette reader. Ultraviolet absorption data was collected on Thermo scientific NANODROP™ 2000c UV-Vis Spectrophotometer.

### Example 1

### Fluorescence Assays

All compounds were purified by HPLC and verified by LC-MS prior to quantitative activation experiments. Stock solutions in methanol were diluted into 1.5 mL of the appropriate solvent in a 1 cm×1 cm quartz cuvette. Measurements of solvent and pre-activation emission spectra for baseline values were made in at least triplicate, prior to addition of trans-cyclooctenol or cyclopropene 4 to initiate the fluorogenic reaction. 3-fold additional excess of trans-cyclooctenol and cyclopropene 4 were added for each fluorogenic reaction. Activation ratios were calculated from the peak emission intensity of the reaction product and the corresponding baseline intensity, and all the intensity data was background subtracted. Integration of the area under the emission intensity curves was used to validate the activation ratios.

For quantum yield determinations, fluorescein in 0.1M NaOH was used as a reference for compounds 5, 6, 9 and 10, with an excitation wavelength of 480 nm (ex slit 2.5 nm); a value of 0.925 was assigned to the quantum yield of fluorescein (Magde et. al, Photochem. Photobiology., 2002, 75(4), 327-334), Rhodamine 6G in EtOH was used as a reference for compounds 7 and 8, with an excitation wavelength of 515 nm (ex slit 2.5 nm); a value of 0.95 (Magde et. al, Photochem. Photobiology., 2002, 75(4), 327-334), and calculations made according to the methods described by Crosby and Demas (Chemical Reviews, 1971, 75(8), 991-1024).

### Example 2

### Live Cell Imaging Studies

### Synthesis of trans-cyclooctene NHS ((E)-cyclooct-4-en-1-yl (2,5-dioxopyrrolidin-1-yl) glutarate)

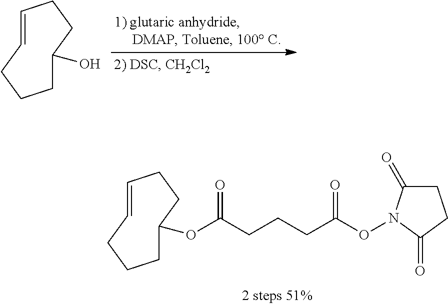 [SMILES: OC1CC/C=C/CCC1 ; O=C(CCCC(=O)ON1C(=O)CCC1=O)OC1CC/C=C/CCC1]

DMAP (6.1 mg, 0.05 mmol) was added to a stirred solution of (E)-cyclooct-4-enol (5.0 mg, 0.040 mmol) in toluene (1.0 mL), followed by glutaric anhydride (6.0 mg, 0.05 mmol). The resulting reaction solution was heated to 100° C. and stirred at this temperature for 18 hours. After TLC indicated that the reaction had finished the solvent was evaporated and the residue was dissolved in CH₂Cl₂, followed by addition of N,N′-disuccinimidyl carbonate (13.0 mg, 0.05 mmol). After stirring at room temperature for 30 minutes, the reaction solution was evaporated and the residue was purified by preparative TLC (Hexanes:Ethyl Acetate (EtOAc)=2:1) to afford 7.0 mg product as a colorless liquid, in 51% yield. ¹H NMR (500 MHz, CDCl₃) δ 1.59-1.71 (2H, m), 1.89-2.05 (6H, m), 2.30-2.40 (6H, m), 2.68 (t, J=10 Hz, 2H), 2.83 (4H, bs), 4.42-4.45 (1H, t, m), 5.46-5.60 (2H, m); ¹³C (100 MHz, CDCl₃) 20.05, 25.80, 30.28, 31.18, 32.72, 33.30, 34.46, 38.81, 41.10, 80.64, 133.27, 135.13, 168.32, 169.27, 171.95; HRMS [M+Na]⁺ m/z calc. for [C₁₇H₂₃NO₆Na]⁺ 360.1418, found 360.1419.

Cell culture and labeling. LS174T cells were grown in DMEM media supplemented with 10% fetal bovine serum, 1% L-glutamine, 1% penicillin/streptomycin. Cells were incubated in 5.0% carbon dioxide, 95% humidity at 37° C. The cells were grown in a T-75 tissue culture flask and seeded on a Lab-Tek II chamber slide two days prior to the experiment. A33 antibody (R&D Systems, MN) was modified by incubation with 50 equivalents of trans-cyclooctene NHS ester for 2 hours at room temperature. The A33 antibody was washed three times with 0.1M sodium bicarbonate buffer pH 8.3 using a 30 kDa spin filter before adding the antibody to the cells at a final concentration of 200 nM for 1 hour.

Image acquisition. All images were acquired on a Yokagawa spinning disk system (Yokagawa, Japan) built around an Axio Observer Zlmotorized inverted microscope (Carl Zeiss Microscopy GmbH, Germany) with a 40×, 1.40 NA oil immersion objective. An Evolve 512×512 EMCCD camera (Photometrics, Canada) was used with ZEN imaging software (Carl Zeiss Microscopy GmbH, Germany). Environmental conditions were maintained at 37° C., 5% CO₂ with a heated enclosure and CO₂ controller (Pecon, Germany). Fluorophores were excited with a 488 nm, 100 MW green OPSL laser. The media was aspirated, and cells were washed twice with PBS before imaging.

### Example 3

### Synthesis of 3-methyl-6-hydroxyethyl-1,2,4,5-tetrazine Tza and 3, 6-hydroxyethyl-1,2, 4,5-tetrazine Tzd

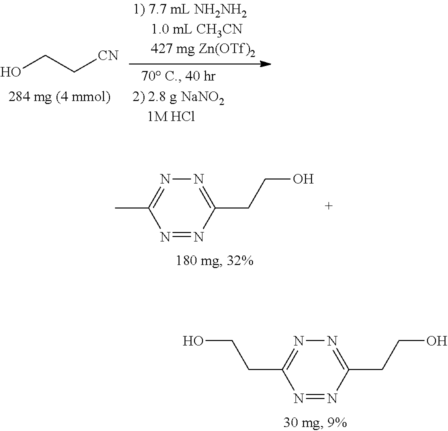

Following a previously developed procedure (Angew. Chem. Int. Ed. 2012, 51, 5222-5225), to a 50 mL flask equipped with a stir bar, Zn(OTf)₂ (427 mg, 1.2 mmol), 3-hydroxy-propionitrile (285 mg, 4 mmol), acetonitrile (1.0 mL, 20 mmol), and anhydrous hydrazine (7.7 mL, 60 mmol) were added. The reaction was protected with a shield. Under the N₂ gas, the mixture was stirred in an oil bath at 70° C. for 40 hours. The reaction solution was cooled with ice water, and sodium nitrite (40 mmol, 2.8 g) dissolved in 20 mL of ice water was slowly added, followed by slow addition of 1M HCl during which time the solution turned bright red, and gas evolved. Addition of 1M HCl continued until gas evolution ceased and the pH value was 3. The mixture was evaporated to remove the water and solvent, and EtOAc was added to the residue, and the solid was filtered. The filtrate was evaporated and the residue was purified by silica column chromatography. 6-methyl-3-hydroxyethyl-s-tetrazine Tza was isolated in 32% yield (Hexane:EtOAc=1:1, 180 mg) and 3,6-dihydroxyethyl-s-tetrazine was isolated in 9% yield (CH₂Cl₂:MeOH=15:1, 30 mg). For applications requiring the absence of trace metals, we suggest washing the organic phase with an aqueous solution of EDTA prior to purification.

### 3,6-dihydroxyethyl-1,2,4,5-tetrazine Tzd

The title product was a red liquid. ¹H NMR (500 MHz, Acetone-d₆) δ 3.46 (t, J=7.5 Hz, 4H), 4.14 (t, J=10 Hz, 4H). ¹³C NMR (125 MHz, Acetone-d₆) 38.4, 60.0, 168.8; HRMS [M+H]⁺ m/z calcd. for [C₆H₁₁N₄O₂]⁺ 171.0877, found 171.0875.

### Example 4

### Synthesis of 3-tert-butyl-6-hydroxyethyl-1, 2,4, 5-tetrazine Tzb

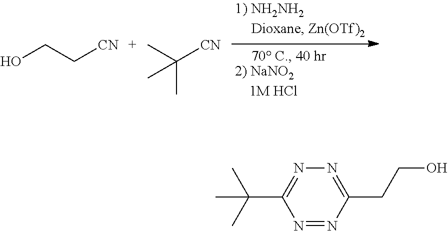

To a 50 mL flask equipped with a stir bar, Zn(OTf)₂ (363 mg, 1.0 mmol), 3-hydroxy-propionitrile (840 mg, 10 mmol), 2,2,2-trimethyl acetonitrile (140 mg, 2 mmol), and anhydrous hydrazine (1.5 mL, 50 mmol) were added. The reaction was protected with a shield. Under N₂ gas, the mixture was stirred in an oil bath at 70° C. for 40 hours. Sodium nitrite (20.0 mmol, 1.4 g) in 20 mL of ice water was slowly added to the solution, followed by slow addition of 1M HCl during which the solution turned bright red in color and gas evolved. Addition of 1M HCl continued until gas evolution ceased and the pH value was 3. The solution was extracted with EtOAc (50 mL×3), the combined organic layer was dried over Na₂SO₄ and evaporated. The residue was purified by silica column (Hexanes:EtOAc=1.5:1) to afford Tzb 150 mg product as pink oil, with a yield of 42%. ¹H NMR (500 MHz, CDCl₃) δ 1.58 (s, 9H), 3.57 (t, J=5 Hz, 2H), 4.27 (t, J=5 Hz, 2H). ¹³C NMR (125 MHz, CDCl₃) δ 29.2, 37.4, 38.0, 60.0, 167.9, 176.2; HRMS [M+H]⁺ m/z calcd. for [C₈H₁₅N₄O]⁺ 183.1240, found 183.1242.

### Example 5

### Synthesis of 3-H-6-hydroxyethyl-1, 2, 4,5-tetrazine Tzc

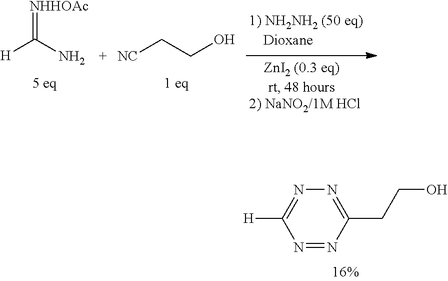

Hydrazine (3.2 g, 100 mmol) was added to a stirring suspension of 3-hydroxy-propionitrile (142 mg, 2 mmol), formamidine acetate (1.01 g, 10 mmol), and ZnI₂ (200 mg, 0.6 mmol) in 1,4-dioxane (3 mL) at room temperature. The reaction was stirred at room temperature for 48 h. Sodium nitrite (20 mmol, 1.4 g) in 20 mL of ice water was slowly added to the solution, followed by slow addition of 1M HCl during which the solution turned bright red in color and gas evolved. Addition of 1M HCl continued until gas evolution ceased and the pH value was 3. The mixture was extracted with EtOAc and the organic phase dried over sodium sulfate. The EtOAc was removed using rotary evaporation and the residue was purified by preparative TLC (CH₂Cl₂:MeOH=30:1) to afford 41 mg compound Tzc as a red oil, with the resulting yield of 16%. ¹H NMR (500 MHz, CDCl₃) δ 3.61-3.64 (m, 2H), 4.29 (bs, 2H), 10.26 (s, 1H); ¹³C (125 MHz, CDCl₃) δ 38.2, 60.1, 158.5, 171.6. HRMS [M+H]⁺ m/z calcd. for [C₄H₇N₄O]⁺ 127.0620, found 127.0618.

### Example 6

### Synthesis of 3-phenyl-6-hydroxyethyl-s-tetrazine Tze

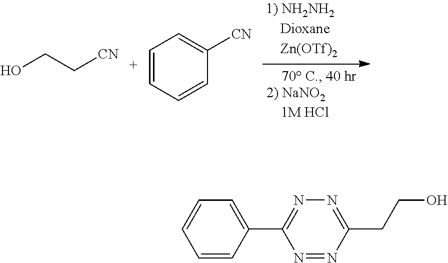

To a 50 mL flask equipped with a stir bar, Zn(OTf)₂ (363 mg, 1.0 mmol), 3-hydroxy-propionitrile (430 mg, 6 mmol), benzonitrile (206 mg, 2 mmol), and anhydrous hydrazine (1.5 mL, 50 mmol), dioxane (1 mL) were added. The reaction was protected with a shield. Under N₂ gas, the mixture was stirred in an oil bath at 70° C. for 40 hours. Sodium nitrite (20 mmol, 1.4 g) in 20 mL of ice water was slowly added to the solution, followed by slow addition of 1M HCl during which the solution turned bright red in color and gas evolved. Addition of 1M HCl continued until gas evolution ceased and the pH value was 3. The solution was extracted with EtOAc (50 mL×3), the combined organic layer was dried over Na₂SO₄ and evaporated. The residue was purified by silica column (Hexanes:EtOAc=1.5:1) to afford 143 mg Tze product as pink solid, with a yield of 36%. ¹H NMR (500 MHz, CDCl₃) δ 8.65-8.50 (m, 2H), 7.70-7.53 (m, 3H), 4.30 (t, J=5.8 Hz, 2H), 3.62 (t, J=5.8 Hz, 2H). ¹³C NMR (126 MHz, CDCl₃) δ 168.66, 164.93, 133.17, 131.92, 129.69, 129.69, 128.37, 128.37, 60.44, 37.85. HRMS [M+H]⁺ m/z calcd. for C₁₀H₁₁N₄O]⁺ 203.0927, found 203.0925.

### Example 7

### Synthesis of 3-(thiophen-3-yl)-6-hydroxyethyl-s-tetrazine Tzf

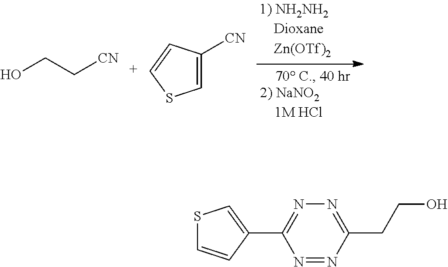

To a 50 mL flask equipped with a stir bar, Zn(OTf)₂ (363 mg, 1.0 mmol), 3-hydroxy-propionitrile (430 mg, 6 mmol), 3-Thiophenecarbonitrile (238 mg, 2 mmol), and anhydrous hydrazine (1.5 mL, 50 mmol), dioxane (1 mL) were added. The reaction was protected with a shield. Under N₂ gas, the mixture was stirred in an oil bath at 70° C. for 40 hours. Sodium nitrite (20.0 mmol, 1.4 g) in 20 mL of ice water was slowly added to the solution, followed by slow addition of 1M HCl during which the solution turned bright red in color and gas evolved. Addition of 1M HCl continued until gas evolution ceased and the pH value was 3. The solution was extracted with EtOAc (50 mL×3), the combined organic layer was dried over Na₂SO₄ and evaporated. The residue was purified by silica column (Hexanes:EtOAc=1.5:1) to afford 147 mg product Tzf as a pink solid, with a yield of 35%. ¹H NMR (500 MHz, CDCl₃) δ 8.56 (dd, J=3.0, 1.2 Hz, 1H), 7.99 (dd, J=5.1, 1.2 Hz, 1H), 7.49 (dd, J=5.1, 3.0 Hz, 1H), 4.26 (t, J=5.9 Hz, 2H), 3.57 (t, J=5.9 Hz, 2H), 3.39-2.70 (br, 1H). ¹³C NMR (126 MHz, CDCl₃) δ 167.54, 161.89, 134.37, 130.12, 127.44, 126.26, 59.90, 37.40. HRMS [M+H]+m/z calcd. for [C₈H₉N₄OS]⁺ 209.02492, found 209.0493.

### Example 8

### Synthesis of 3-(2-tert-Butoxycarbonylaminoethyl)-6-hydroxyethyl-s-tetrazine Tzg

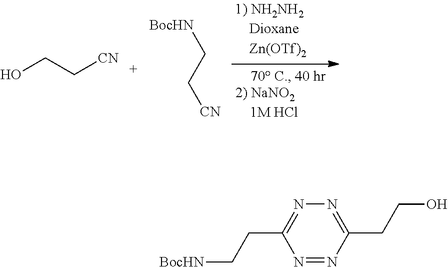

To a 50 mL flask equipped with a stir bar, Zn(OTf)₂ (363 mg, 1.0 mmol), 3-hydroxy-propionitrile (430 mg, 6 mmol), tert-Butyl-2-cyanoethylcarbamate (340 mg, 2 mmol), and anhydrous hydrazine (1.5 mL, 50 mmol), dioxane (lmL) were added. The reaction was protected with a shield. Under N₂ gas, the mixture was stirred in an oil bath at 70° C. for 40 hours. Sodium nitrite (20.0 mmol, 1.4 g) in 20 mL of ice water was slowly added to the solution, followed by slow addition of 1M HCl during which the solution turned bright red in color and gas evolved. Addition of 1M HCl continued until gas evolution ceased and the pH value was 3. The solution was extracted with EtOAc (50 mL×3), the combined organic layer was dried over Na₂SO₄ and evaporated. The residue was purified by silica column (Hexanes:EtOAc=1.5:1) to afford 318 mg product Tzg as a pink solid, with a yield of 59%. ¹H NMR (500 MHz, CDCl₃) δ 5.15 (s, 1H), 4.22 (t, J=5.8 Hz, 2H), 3.69 (d, J=6.0 Hz, 2H), 3.54 (t, J=5.8 Hz, 2H), 3.52-3.44 (m, 2H), 2.93 (br, 1H), 1.34 (s, 9H). ¹³C NMR (126 MHz, CDCl₃) δ 168.66, 168.59, 156.00, 79.77, 60.16, 38.62, 37.69, 35.73, 28.39. HRMS [M+Na]+m/z calcd. for [C₁₁H₁₉N₃O₃Na]⁺ 292.1380, found 292.1382.

### Example 9

### General Procedure for Synthesis of 1a-1g

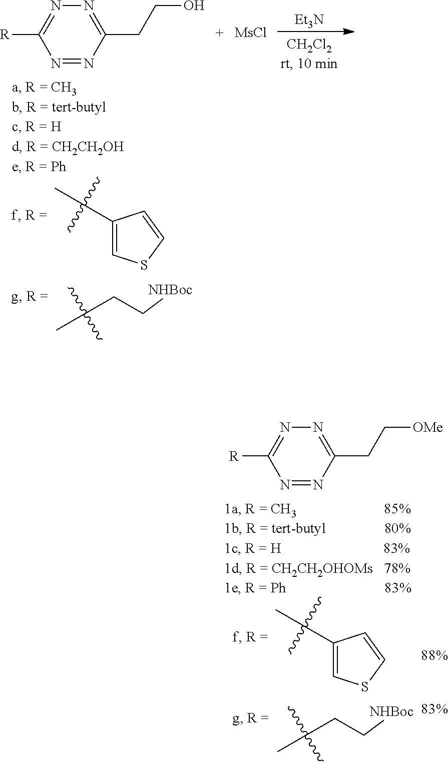

In a 50 mL flask, 1,2,4,5-Tetrazines Tza-Tzg (1.0 eq) were dissolved in CH₂Cl₂, with added Et₃N (1.2 eq), followed by the addition of MsCl (1.2 eq). The reaction solution was stirred at room temperature for 10 min, and checked for completion by TLC. The reaction solution was washed with water. The organic layer was dried over Na₂SO₄ and evaporated. The residues were purified by silica column chromatography to afford products 1a-1g.

Cmpd 1a:

180 mg of starting material Tza yields 238 mg 1a as a red solid after silica column chromatography (Hexane: EtOAc=1:1). Yield: 85%. ¹H NMR (500 MHz, CDCl₃) δ 2.99 (s, 3H), 3.04 (s, 3H), 3.73 (t, J=7.5 Hz, 2H), 4.83 (dt, J=10, 5 Hz, 2H). ¹³C NMR (125 MHz, CDCl₃) δ 21.4, 34.7, 37.7, 66.4, 166.3, 168.4; HRMS [M+H]⁺ m/z calcd. for [C₆H₁₁N₄O₃S]⁺ 219.0546, found 219.0550.

Cmpd 1b:

50 mg of starting material Tzb affords 57 mg of 1b as a red solid after silica column chromatography (Hexane: EtOAc=2:1). Yield: 80%. ¹H NMR (500 MHz, CDCl₃) δ 1.55 (s, 9H), 3.01 (s 3H), 3.73-3.76 (m, 2H), 4.84-4.87 (m, 2H). ¹³C NMR (125 MHz, CDCl₃) δ 29.2, 34.6, 37.6, 38.1, 66.2, 165.6, 176.4; HRMS [M+Na]⁺ m/z calcd. for [C₉H₁₆N₄O₃SNa]+283.0835, found 283.0838.

Cmpd 1c:

30 mg of starting material Tzc affords 40 mg of 1c as a red solid after silica column chromatography (Hexane: EtOAc=1:1). Yield: 83%. ¹H NMR (500 MHz, CDCl₃) δ 3.01 (s, 3H), 3.81 (t, J=10 Hz, 2H), 4.87 (t, J=7.5 Hz, 2H), 10.27 (s, 1H). ¹³C NMR (125 MHz, CDCl₃) δ 35.3, 37.7, 66.0, 158.7, 169.4; HRMS [M+Na]⁺ m/z calcd. for [C₅H₈N₄O₃SNa]⁺ 227.0209, found 227.0210.

Cmpd 1d:

25 mg of starting material Tzd affords 37 mg of 1d as a red solid after silica column chromatography (Hexane: EtOAc=1:1). Yield: 78%. ¹H NMR (500 MHz, Acetone-d₆) δ 3.10 (s, 6H), 3.81 (t, J=7.5 Hz, 4H), 4.89 (t, J=7.5 Hz, 4H); ¹³C NMR (125 MHz, Acetone-d₆) 34.7, 36.5, 67.2, 167.5; HRMS [M+Na]+m/z calcd. For [CsH₁₄N₄O₆S₂Na]⁺ 349.0247, found 349.0251.

Cmpd 1e:

20 mg of starting material Tze affords 24 mg of 1e as a red solid after silica column chromatography (Hexane: EtOAc=1:1). Yield: 83%. ¹H NMR (500 MHz, CDCl₃) δ 8.69-8.55 (m, 2H), 7.62 (ddd, J=13.1, 7.9, 6.3 Hz, 3H), 4.92 (t, J=6.2 Hz, 2H), 3.83 (t, J=6.2 Hz, 2H), 3.04 (s, 3H). ¹³C NMR (126 MHz, CDCl₃) δ 165.95, 164.66, 132.88, 131.27, 129.24, 129.24, 128.04, 128.04, 65.86, 37.47, 34.51. HRMS [M+Na]⁺ m/z calcd. For [C₁₁H₁₂N₄O₃SNa]⁺ 303.0522, found 303.0524.

Cmpd 1f:

21 mg of starting material Tzf affords 25 mg of 1f as a red solid after silica column chromatography (Hexane: EtOAc=1:1). Yield: 88%. ¹H NMR (500 MHz, CDCl₃) δ 8.63 (dd, J=3.0, 1.2 Hz, 1H), 8.05 (dd, J=5.1, 1.2 Hz, 1H), 7.53 (dd, J=5.1, 3.0 Hz, 1H), 4.90 (t, J=6.2 Hz, 2H), 3.79 (t, J=6.2 Hz, 2H), 3.04 (s, 3H). ¹³C NMR (126 MHz, CDCl₃) δ 165.64, 162.45, 134.57, 130.86, 127.84, 126.67, 66.13, 37.73, 34.80. HRMS [M+Na]⁺ m/z calcd. For [C₉H₁₀N₄O₃S₂Na]⁺ 309.0087, found 309.0089.

Cmpd 1g:

27 mg of starting material Tzf affords 29 mg of 1g as a red solid after silica column chromatography (Hexane: EtOAc=1:1). Yield: 83%. ¹H NMR (500 MHz, CDCl₃) δ 5.07 (s, 1H), 4.84 (t, J=5.5 Hz, 2H), 3.74 (t, J=5.5 Hz, 2H), 3.69 (d, J=5.8 Hz, 2H), 3.50 (t, J=5.3 Hz, 2H), 3.00 (s, 3H), 1.33 (s, 9H). ¹³C NMR (126 MHz, CDCl₃) δ 169.00, 166.42, 155.87, 79.61, 66.15, 38.55, 37.53, 35.72, 34.65, 28.36. HRMS [M+Na]m/z calcd. For [C₁₂H₂₁N₅O₅SNa]⁺ 370.1156, found 370.1158.

### Example 10

### General Procedure for Screening the Back Reaction Conditions

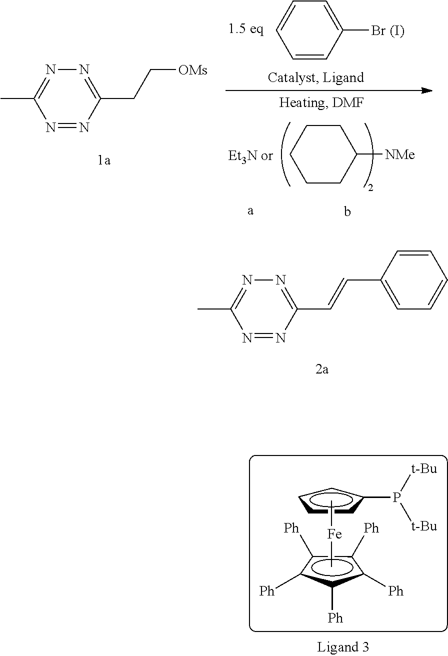

To a 10 mL microwave reaction tube equipped with a stir bar, catalysts (0.03-0.1 eq) and ligands (0.12-0.4 eq), 1a (1 eq, 6.0 mg, 0.0276 mmol), iodobenzene (1.5 eq, 8.4 mg, 0.0414 mmol) or bromobenzene (1.5 eq, 6.5 mg, 0.0414 mmol), and base (3.0 eq, 0.0828 mmol) were dissolved in anhydrous DMF (1.5 mL). The reaction was protected with N₂ gas and then heated 5 in an oil bath (90° C., 90 min) or by microwave irradiation (50-60° C., 30-45 min). The reaction solution was cooled to room temperature and EtOAc (20 mL) was added before washing with water (20 mL×3). The organic layer was dried over Na₂SO₄ and evaporated. The residue was purified by a TLC plate (Agela Technologies, silica 200×200 mm, PH=5, MF=254, glass back; Hexanes:Et₂O=3:1) to afford pure 2a as a pink solid. ¹H NMR (500 MHz, CDCl₃) δ 3.05 (s, 3H), 7.44 (m, 4H), 7.68 (t, J=2.5 Hz, 2H), 8.31 (d, J=20 Hz, 1H). ¹³C NMR (125 MHz, CDCl₃) δ 21.3, 120.6, 128.2, 129.1, 130.3, 135.2, 141.1, 164.8, 166.4; HRMS [M+H]⁺ m/z calcd. for [C₁₁H₁₁N₄]⁺ 199.0978, found 199.0976.

### Example 11

### Synthesis of 4-bromo-N-Boc-DL-phenylalanine methyl ester and 5-bromo-N₂-Boc-DL-tryptophan methyl ester

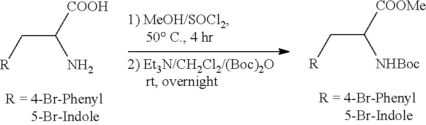

To a 50 mL flask equipped with a stir bar, the amino acid (0.25 mmol) was dissolved in methanol (10 mL) and cooled by ice-water. SOCl₂ (90 mg, 0.75 mmol) was added dropwise to the solution and then warmed to 50° C. The reaction was stirred at this temperature for 4 hours and then evaporated to afford the amino acid methyl ester hydrochloride as a white solid. The amino acid methyl ester hydrochloride (0.25 mmol) was dissolved in CH₂Cl₂ (20 mL), and Et₃N (0.6 mmol) was added, followed by (Boc)₂O (65 mg, 0.3 mmol). The resulting solution was stirred at room temperature overnight and then washed with brine (20 mL). The organic layer was dried over Na₂SO₄ and evaporated. The residue was purified by silica column chromatography to afford 4-bromo-N-boc-DL-phenylalanine methyl ester (Hexanes:EtOAc=2:1, 75 mg, 85% yield) and 5-bromo-N₂-boc-DL-tryptophan methyl ester (Hexanes: EtOAc=1:1, 90 mg, 91% yield).

### 4-bromo-N-Boc-DL-phenylalanine methyl ester

¹H NMR (400 MHz, CDCl₃) δ 1.40 (s, 9H), 2.97 (dd, J=12, 8 Hz, 1H), 3.07 (dd, J=12, 8 Hz, 1H), 3.70 (s, 3H), 4.56 (t, J=6 Hz, 1H), 5.00 (d, J=6 Hz, 1H), 6.99 (d, J=4 Hz, 2H), 7.40 (d, J=4 Hz, 2H). ¹³C NMR (100 MHz, CDCl₃) δ 28.4, 37.9, 52.4, 54.3, 80.2, 121.1, 131.1, 131.7, 135.2, 155.1, 172.2; HRMS [M+Na]⁺ m/z calcd. for [C₁₅H₂₀BrNO₄Na]⁺ 397.0763, found 397.0760.

### 5-bromo-N₂-Boc-DL-tryptophan methyl ester

¹H NMR (400 MHz, CDCl₃) δ 1.44 (s, 9H), 3.24 (bs, 2H), 3.70 (s, 3H), 4.64 (d, J=8 Hz, 1H), 5.11 (d, J=4 Hz, 1H), 6.97 (s, 1H), 7.18-7.24 (m, 2H), 7.65 (s, 1H), 8.33 (s, 1H); ¹³C NMR (100 MHz, CDCl₃) δ 28.2, 28.6, 52.6, 54.4, 80.3, 110.2, 112.9, 113.1, 121.7, 124.3, 125.2, 129.7, 134.9, 155.4, 172.7. HRMS [M+H]⁺ m/z calcd. for [C₁₇H₂₂BrN₂O₄]⁺ 380.0468, found 380.0470.

### Example 12

### Synthesis of 5-bromo-N, N₂—Boc-DL-tryptophan methyl ester

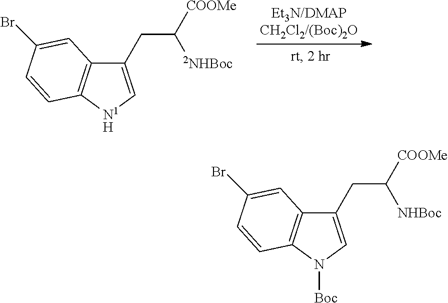

In a 50 mL flask, 5-bromo-N₂-Boc-DL-tryptophan methyl ester (50 mg, 0.126 mmol)

was dissolved in CH₂Cl₂, and Et₃N (13 mg, 0.13 mmol), DMAP (1.5 mg, 0.0126 mmol) was added, and then followed by (Boc)₂O (33 mg, 0.15 mmol). The reaction solution was stirred at room temperature for 2 hours and then washed with brine. The organic layer was dried over Na₂SO₄ and evaporated. The residue was purified with silica column chromatography and afforded 58 mg 5-bromo-N₁,N₂-boc-DL-tryptophan methyl ester as a white solid at 92%. yield. ¹H NMR (500 MHz, CDCl₃) δ 1.44 (s, 9H), 1.65 (s, 9H), 3.13 (dd, J=15, 10 Hz, 1H), 3.23 (dd, J=15, 5 Hz, 1H), 3.72 (s, 3H), 4.62 (t, J=2.5 Hz, 1H), 5.14 (d, J=10 Hz, 1H), 7.37 (s, 1H), 7.58 (d, J=5 Hz, 2H), 7.97 (bs, 1H). ¹³C NMR (100 MHz, CDCl₃) δ 28.0, 28.4, 28.6, 52.7, 53.9, 84.4, 114.7, 116.2, 117.0, 122.0, 125.5, 127.5, 132.5, 134.3, 149.4, 155.2, 172.3; HRMS [M+Na]⁺ m/z calcd. for

### Example 13

### Synthesis of 3-bromo-2′,7′-difluorofluorescein (3-bromo-Oregon Green 488) and 4-bromo-2′,7′-difluorofluorescein (4-bromo-Oregon Green 488)

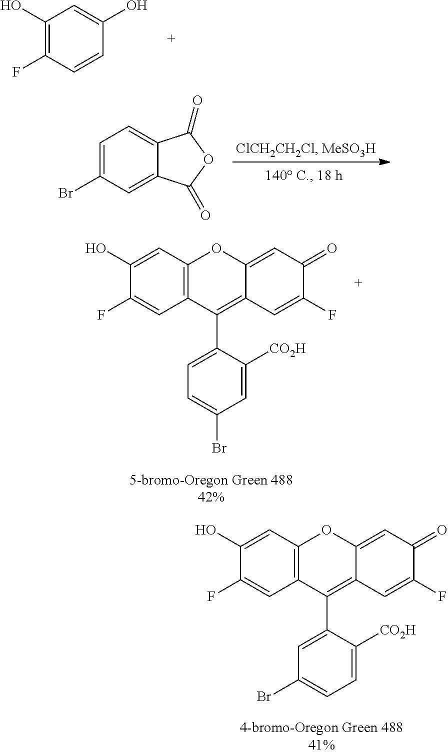

A mixture of 4-Fluoro-1, 3-dihydroxybenzene (32 mg, 0.25 mmol), 4-bromophthalic anhydride (28 mg, 0.123 mmol), 1,2-dichloroethane (1 mL) and methanesulfonic acid (2 mL) was heated at 140° C. in seal tube for 18 h. The resultant dark yellow solution was dissolved in EtOAc (100 mL), and washed with water and saturated sodium chloride over Na₂SO₄. The solution was concentrated to give a crude mixture of 3-bromo-Oregon Green 488 and 4-bromo-Oregon Green 488. The isomers were separated by prep-HPLC. Results: 3-bromo-Oregon Green 488 23 mg as a yellow solid, yield 42%; 4-bromo-Oregon Green 488 21 mg as a yellow solid, yield 41%.

### 3-bromo-Oregon Green 488

¹H NMR (500 MHz, CD₃OD) δ 7.94 (d, J=8.2 Hz, 1H), 7.89 (d, J=8.1 Hz, 1H), 7.45 (s, 1H), 6.84 (d, J=7.2 Hz, 2H), 6.46 (d, J=10.9 Hz, 2H). ¹³C NMR (126 MHz, CD₃OD) δ 168.38, 149.79, 148.68, 147.87, 133.79, 130.10, 127.54, 126.97, 125.81, 113.11, 112.95, 108.57, 104.82. HRMS [M+Na]⁺ m/z calcd. for [C₂₀H₉BrF₂O₅Na]⁺ 468.9494, found 468.9486.

### 4-bromo-Oregon Green 488

¹H NMR (500 MHz, DMSO-D6) δ 10.79 (s, 2H), 8.14 (d, J=1.4 Hz, 1H), 7.95 (dd, J=8.2, 1.7 Hz, 1H), 7.26 (d, J=8.2 Hz, 1H), 6.87 (d, J=7.5 Hz, 2H), 6.65 (d, J=11.3 Hz, 2H). ¹³C NMR (126 MHz, DMSO-D6) δ 167.40, 151.08, 149.39, 148.28, 148.17, 147.84, 147.47, 138.90, 128.92, 128.31, 126.49, 123.91, 114.75, 114.58, 108.24, 108.19, 105.25. HRMS [M+Na]⁺ m/z calcd. for [C₂₀H₉BrF₂O₅Na]⁺ 468.9494, found 468.9488.

### Example 14

### General procedure for the synthesis of (E)-3-substituted-6-alkenyl-1,2,4,5-tetrazines 2b-2x

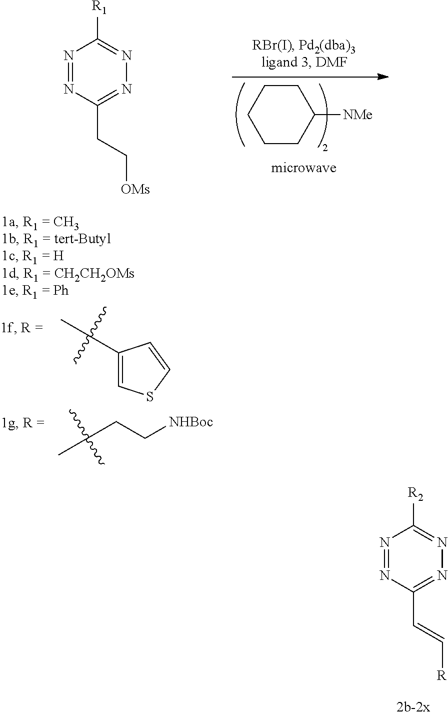

To a 10 mL microwave reaction tube equipped with a stir bar, catalysts (0.03-0.05 eq) and ligand 3 (0.12-0.2 eq), 1a-1g (1 eq), RBr (RI) (1.1-1.5 eq) and N,N-dicyclohexylmethylamine (3.0-6.0 eq) were dissolved in anhydrous DMF (1.5 mL). The reaction was protected with N₂ gas and then heated by microwave irradiation (50° C., 30-60 min). The reaction solution was cooled to room temperature and EtOAc (20 mL) was added before washing with water (20 mL×3). The organic layer was dried over Na₂SO₄ and evaporated. The residue was purified by preparative-TLC to afford purified compounds 2b-2x.

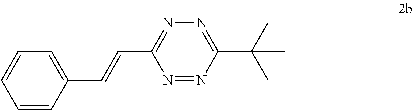 [SMILES: CC(C)(C)c1nnc(/C=C/c2ccccc2)nn1]

Cmpd 2b:

1.5 eq bromobenzene, 3.0 eq base, Pd₂(dba)₃(3%), ligand 3 (12%), microwave irradiation at 50° C. for 30 min. 6 mg 1b affords 5.37 mg 2b as a pink liquid, in 97% yield. ¹H NMR (500 MHz, CDCl₃) δ 1.60 (s, 9H), 7.41-7.49 (m, 4H), 7.70 (t, J=5 Hz, 2H), 8.39 (d, J=15 Hz, 1H). ¹³C NMR (125 MHz, CDCl₃) δ 29.1, 37.8, 120.5, 127.9, 128.9, 135.0, 130.0, 140.7, 164.1, 174.5. HRMS [M+H]⁺ m/z calcd. for [C₁₄H₁₇N₄]⁺ 241.1448, found 241.1445.

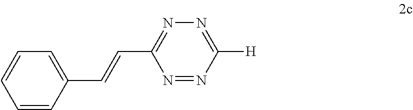 [SMILES: C(=C\c1nncnn1)/c1ccccc1]

Cmpd 2c:

1.5 eq bromobenzene, 3.0 eq base, Pd₂(dba)₃ (5%), ligand 3 (20%), m irradiation at 50° C. for 30 min. 5.7 mg 1c affords 3.75 mg 2c as a pink solid, in 83% yield. ¹H NMR (500 MHz, CDCl₃) δ 7.45-7.50 (m, 4H), 7.71 (dd, J=5, 2 Hz, 2H), 8.39 (d, J=15 Hz, 1H), 10.1 (s, 1H. ¹³C NMR (125 MHz, CDCl₃) δ 120.5, 128.4, 129.2, 130.7, 134.9, 142.7, 157.0, 167.3; HRMS [M+H]⁺ m/z calcd. for [C₁₀H₉N₄]⁺ 185.0822, found 185.0825.

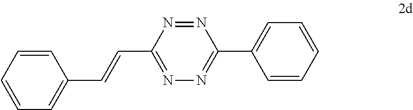 [SMILES: C(=C\c1nnc(-c2ccccc2)nn1)/c1ccccc1]

Cmpd 2d:

1.5 eq bromobenzene, 3.0 eq base, Pd₂(dba)₃ (3%), ligand 3 (12%), microwave irradiation at 50° C. for 35 min. 5.6 mg 1e affords 5.2 mg 2d as a pink solid, in 99% yield. ¹H NMR (500 MHz, CDCl₃) δ 8.65-8.60 (m, 2H), 8.38 (d, J=16.3 Hz, 1H), 7.72 (dd, J=8.2, 1.3 Hz, 2H), 7.64-7.58 (m, 3H), 7.54 (d, J=16.3 Hz, 1H), 7.45 (ddd, J=8.5, 6.4, 3.5 Hz, 3H). ¹³C NMR (126 MHz, CDCl₃) δ 164.84, 163.20, 141.33, 135.29, 132.69, 132.04, 130.46, 129.42, 129.42, 129.20, 129.20, 128.30, 128.30, 128.04, 128.04, 120.75; HRMS [M+H]⁺ m/z calcd. for [C₁₆H₁₃N₄]⁺ 261.1135, found 261.1162.

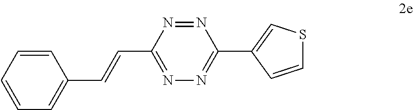 [SMILES: C(=C\c1nnc(-c2ccsc2)nn1)/c1ccccc1]

Cmpd 2e:

1.5 eq bromobenzene, 3.0 eq base, Pd₂(dba)₃ (3%), ligand 3 (12%), microwave irradiation at 50° C. for 35 min. 5.7 mg 1f affords 4.3 mg 2e as a pink solid, in 82% yield. ¹H NMR (500 MHz, CDCl₃) δ 8.61 (dd, J=3.0, 1.2 Hz, 1H), 8.33 (d, J=16.3 Hz, 1H), 8.07 (dd, J=5.1, 1.2 Hz, 1H), 7.73-7.68 (m, 2H), 7.53 (dd, J=5.2, 3.2 Hz, 1H), 7.50 (d, J=16.3 Hz, 1H), 7.48-7.41 (m, 3H). ¹³C NMR (126 MHz, CDCl₃) δ 164.37, 160.87, 140.98, 135.32, 135.16, 130.40, 130.05, 129.19, 129.19, 128.26, 128.26, 127.63, 126.65, 120.84. HRMS [M+H]⁺ m/z calcd. for [C₁₄H₁₁N₄S]⁺ 267.0699, found 267.0700.

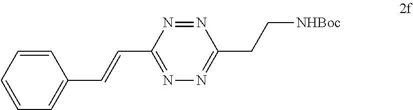 [SMILES: CC(C)(C)OC(=O)NCCc1nnc(/C=C/c2ccccc2)nn1]

Cmpd 2f:

1.5 eq bromobenzene, 3.0 eq base, Pd₂(dba)₃ (3%), ligand 3 (12%), microwave irradiation at 50° C. for 45 min. 6.9 mg 1g affords 5.9 mg 2f as a pink solid, in 91% yield. ¹H NMR (500 MHz, CDCl₃) δ 8.33 (d, J=16.3 Hz, 1H), 7.69 (dd, J=7.9, 1.4 Hz, 2H), 7.50-7.42 (m, 4H), 5.05 (s, 1H), 3.81-3.71 (m, 2H), 3.52 (t, J=6.1 Hz, 2H), 1.39 (s, 9H). ¹³C NMR (126 MHz, CDCl₃) δ 167.31, 165.17, 155.92, 141.51, 135.18, 130.48, 129.20, 128.27, 120.59, 79.72, 38.45, 35.63, 28.46. HRMS [M+Na]⁺ m/z calcd. for [C₁₇H₂₁N₅O₂Na]⁺ 350.1587, found 350.1588.

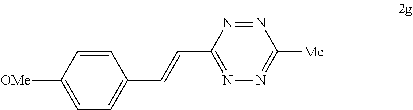 [SMILES: COc1ccc(/C=C/c2nnc(C)nn2)cc1]

Cmpd 2g:

1.2 eq 4-Bromoanisole, 3.0 eq base, Pd₂(dba)₃ (3%), ligand 3 (12%), microwave irradiation at 50° C. for 35 min. 4.4 mg 1a affords 4.2 mg 2g as a pink solid, in 93% yield. ¹H NMR (500 MHz, CDCl₃) δ 8.26 (d, J=16.2 Hz, 1H), 7.63 (d, J=8.6 Hz, 2H), 7.31 (d, J=16.2 Hz, 1H), 6.96 (d, J=8.8 Hz, 2H), 3.86 (s, 3H), 3.03 (s, 3H). ¹³C NMR (126 MHz, CDCl₃) δ 166.13, 165.11, 161.49, 140.75, 129.87, 129.87, 128.04, 118.19, 114.61, 114.61, 55.58, 21.29. HRMS [M+H]⁺ m/z calcd. for [C₁₂H₁₃N₄O]⁺ 229.1084, found 229.1080.

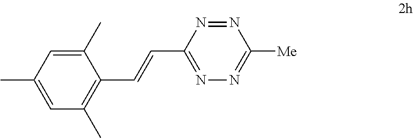 [SMILES: Cc1cc(C)c(/C=C/c2nnc(C)nn2)c(C)c1]

Cmpd 2h:

1.2 eq 2-Bromomesitylene, 3.0 eq base, Pd₂(dba)₃ (3%), ligand 3 (12%), microwave irradiation at 50° C. for 40 min. 4.4 mg 1a affords 4.8 mg 2h as a pink solid, in 97% yield. ¹H NMR (500 MHz, CDCl₃) δ 8.47 (d, J=16.6 Hz, 1H), 7.08 (d, J=16.6 Hz, 1H), 6.95 (s, 2H), 3.06 (s, 3H), 2.44 (s, 6H), 2.05 (s, 3H). ¹³C NMR (126 MHz, CDCl₃) δ 166.63, 164.67, 139.90, 138.63, 137.21, 137.21, 131.71, 129.49, 125.49, 21.47, 21.31, 21.24, 21.21. HRMS [M+H]⁺ m/z calcd. for [C₁₄H₁₇N₄]⁺ 241.1448, found 241.1449.

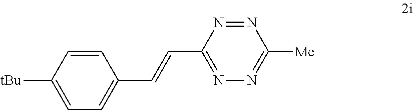 [SMILES: Cc1nnc(/C=C/c2ccc(C(C)(C)C)cc2)nn1]

Cmpd 2i:

1.5 eq 1-Bromo-4-tert-butylbenzene, 3.0 eq base, Pd₂(dba)₃ (3%), ligand 3 (12%), microwave irradiation at 50° C. for 30 min. 4.4 mg 1a affords 4.8 mg 2i as a pink solid, in 96% yield. ¹H NMR (500 MHz, CDCl₃) δ 8.30 (d, J=16.2 Hz, 1H), 7.63 (d, J=8.3 Hz, 2H), 7.47 (d, J=8.4 Hz, 2H), 7.42 (d, J=16.3 Hz, 1H), 3.05 (s, 3H), 1.35 (s, 9H). ¹³C NMR (126 MHz, CDCl₃) δ 166.32, 165.02, 153.97, 141.03, 132.50, 128.06, 128.06, 126.16, 126.16, 119.76, 35.08, 31.32, 21.32. HRMS [M+H]⁺ m/z calcd. for [C₁₅H₁₉N₄]⁺ 255.1604, found 255.1606.

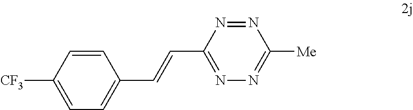 [SMILES: Cc1nnc(/C=C/c2ccc(C(F)(F)F)cc2)nn1]

Cmpd 2j:

1.2 eq 4-Bromobenzotrifluoride, 3.0 eq base, Pd₂(dba)₃ (3%), ligand 3 (12%), microwave irradiation at 50° C. for 40 min. 4.4 mg 1a affords 4.1 mg 2j as a pink solid, in 86% yield. ¹H NMR (500 MHz, CDCl₃) δ 8.33 (d, J=16.3 Hz, 1H), 7.79 (d, J=8.1 Hz, 2H), 7.71 (d, J=8.3 Hz, 2H), 7.54 (d, J=16.3 Hz, 1H), 3.08 (s, 3H). ¹³C NMR (126 MHz, CDCl₃) δ 166.81, 164.47, 143.45, 139.16, 130.62, 129.07, 128.50, 128.25, 128.25, 126.10 (q, J=3.9 Hz), 123.15, 21.36. HRMS [M+H]⁺ m/z calcd. for [C₁₂H₉F₃N₄]⁺ 267.0852, found 267.0858.

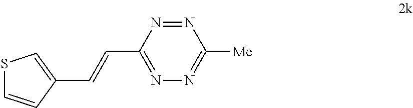 [SMILES: Cc1nnc(/C=C/c2ccsc2)nn1]

Cmpd 2k:

1.4 eq 3-Bromothiophene, 3.0 eq base, Pd₂(dba)₃ (3%), ligand 3 (12%), microwave irradiation at 50° C. for 40 min. 4.4 mg 1a affords 3.5 mg 2k as a pink solid, in 86% yield. ¹H NMR (500 MHz, CDCl₃) δ 8.30 (d, J=16.2 Hz, 1H), 7.62-7.59 (m, 1H), 7.49 (dd, J=5.1, 1.0 Hz, 1H), 7.41 (ddd, J=5.1, 2.9, 0.5 Hz, 1H), 7.28 (d, J=15.0 Hz, 1H), 3.04 (s, 3H). ¹³C NMR (126 MHz, CDCl₃) δ 166.37, 165.07, 138.49, 134.70, 128.17, 127.32, 125.20, 120.40, 21.31. HRMS [M+H]⁺ m/z calcd. for [C₉H₉N₄S]⁺ 205.0542, found 205.0544.

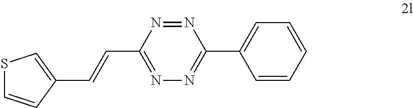 [SMILES: C(=C\c1nnc(-c2ccccc2)nn1)/c1ccsc1]

Cmpd 21:

1.4 eq 3-Bromothiophene, 3.0 eq base, Pd₂(dba)₃ (3%), ligand 3 (12%), microwave irradiation at 50° C. for 40 min. 5.6 mg 1e affords 4.4 mg 21 as a pink solid, in 82% yield. H NMR (500 MHz, CDCl₃) δ 8.61 (dd, J=7.8, 1.7 Hz, 2H), 8.36 (d, J=16.1 Hz, 1H), 7.66-7.57 (m, 4H), 7.53-7.50 (m, 1H), 7.43 (dd, J=5.1, 2.9 Hz, 1H), 7.34 (d, J=16.1 Hz, 1H). ¹³C NMR (126 MHz, CDCl₃) δ 165.03, 163.14, 138.61, 134.88, 132.63, 132.08, 129.40, 129.40, 128.38, 127.99, 127.99, 127.37, 125.25, 120.51. HRMS [M+H]⁺ m/z calcd. for [C₁₄H₁₁N₄S]⁺ 267.0699, found 267.0698.

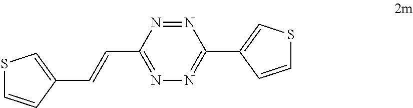 [SMILES: C(=C\c1nnc(-c2ccsc2)nn1)/c1ccsc1]

Cmpd 2m:

1.4 eq 3-Bromothiophene, 3.0 eq base, Pd₂(dba)₃ (3%), ligand 3 (12%), microwave irradiation at 50° C. for 40 min. 5.7 mg 1f affords 2.8 mg 2m as a pink solid, in 58% yield. ¹H NMR (500 MHz, CDCl₃) δ 8.60 (dd, J=3.1, 1.2 Hz, 1H), 8.35-8.29 (m, 1H), 8.07 (dd, J=5.1, 1.2 Hz, 1H), 7.62 (dd, J=1.7, 1.2 Hz, 1H), 7.53 (dd, J=5.1, 3.0 Hz, 1H), 7.51 (dd, J=5.1, 1.0 Hz, 1H), 7.44-7.40 (m, 1H), 7.31 (d, J=16.1 Hz, 1H). ¹³C NMR (126 MHz, CDCl₃) δ 164.56, 160.83, 138.63, 135.19, 134.55, 129.91, 128.26, 127.60, 127.35, 126.63, 125.24, 120.61. HRMS [M+H]⁺ m/z calcd. for [C₁₂H9N₄S_{2]}⁺ 273.0263, found 273.0264.

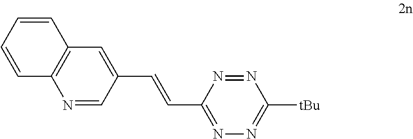 [SMILES: CC(C)(C)c1nnc(/C=C/c2cnc3ccccc3c2)nn1]

Cmpd 2n:

1.1 eq 3-Bromoquinoline, 3.0 eq base, Pd₂(dba)₃ (3%), ligand 3 (12%), microwave irradiation at 50° C. for 40 min. 5.7 mg 1c affords 2.3 mg 2n as a pink solid, in 79% yield. ¹H NMR (500 MHz, CDCl₃) δ 9.26 (d, J=2.1 Hz, 1H), 8.46 (d, J=16.4 Hz, 1H), 8.40 (d, J=1.9 Hz, 1H), 8.15 (d, J=8.4 Hz, 1H), 7.91 (d, J=8.1 Hz, 1H), 7.78 (ddd, J=8.4, 6.9, 1.4 Hz, 1H), 7.71 (d, J=16.4 Hz, 1H), 7.62 (ddd, J=8.1, 6.9, 1.1 Hz, 1H), 1.61 (s, 9H). ¹³C NMR (126 MHz, CDCl₃) δ 165.19, 164.00, 137.30, 135.55, 131.72, 130.87, 129.42, 129.37, 128.50, 128.27, 127.88, 127.72, 122.84, 38.10, 29.28. HRMS [M+H]+m/z calcd. for [C₁₇H₁₈N₅]⁺292.1557, found 292.1560.

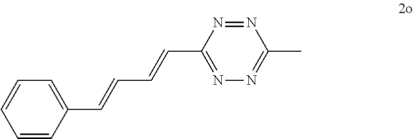 [SMILES: Cc1nnc(/C=C/C=C/c2ccccc2)nn1]

Cmpd 2o:

1.1 eq β-bromostyrene, 3.0 eq base, Pd₂(dba)₃ (5%), ligand 3 (20%), microwave irradiation at 55° C. for 35 min. 6.0 mg 1a affords 2.91 mg 2s as a red solid, in 48% yield. ¹H NMR (500 MHz, CDCl₃) δ 3.03 (s, 3H), 6.98-7.01 (m, 2H), 7.10 (dd, J=15, 10 Hz, 1H), 7.25-7.34 (m, 3H), 7.53 (t, J=5 Hz, 2H), 8.07 (dd, J=15, 10 Hz, 1H). ¹³C NMR (125 MHz, CDCl₃) 21.3, 123.9, 127.3, 127.4, 129.0, 129.2, 136.2, 140.2, 141.3, 164.9, 166.0; HRMS [M+H]⁺ m/z calcd. for [C₁₃H₁₃N₄]+225.1135, found 225.1137.

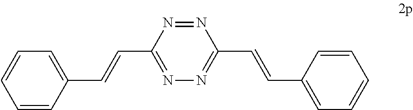 [SMILES: C(=C\c1nnc(/C=C/c2ccccc2)nn1)/c1ccccc1]

Cmpd 2p:

3.0 eq bromobenzene, 6.0 eq base, Pd₂(dba)₃ (6%), ligand 3 (24%), microwave at 50° C. for 30 min. 4.3 mg 1d affords 3.4 mg 2p as a orange solid, in 89% yield. ¹H NMR (500 MHz, CDCl₃) δ 7.42-7.51 (m, 8H), 7.71 (d, J=5 Hz, 4H), 8.33 (d, J=20 Hz, 2H). ¹³C NMR (125 MHz, CDCl₃) δ 120.9, 128.2, 129.2, 130.4, 135.3, 141.0, 163.9; HRMS [M+H]-m/z calcd. for [C₁₈H₁₅N₄]⁺ 287.1291, found 287.1289.

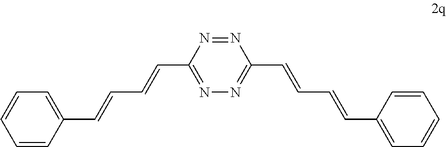 [SMILES: C(/C=C/c1nnc(/C=C/C=C/c2ccccc2)nn1)=C\c1ccccc1]

Cmpd 2q:

2.2 eq β-bromostyrene, 6.0 eq base, Pd₂(dba)₃ (10%), ligand 3 (40%), microwave at 55° C. for 35 min. 5.0 mg 1d afford 3.0 mg 2q as an orange solid, in 58% yield. ¹H NMR (500 MHz, CDCl₃) δ 6.99-7.03 (dd, J=15, 5 Hz, 4H), 7.10-7.15 (dd, J=20, 10 Hz, 2H), 7.32-7.34 (m, 2H), 7.37-7.40 (m, 4H), 7.53 (t, J=5 Hz, 4H), 8.07 (dd, J=17.5, 10 Hz, 2H). ¹³C NMR (125 MHz, CDCl₃) δ 124.4, 127.4, 127.7, 129.0, 129.2, 136.3, 140.2, 141.0, 163.6; HRMS [M+H]⁺ m/z calcd. for [C₂₂H₁₉N₄]⁺ 339.1604, found 339.1603.

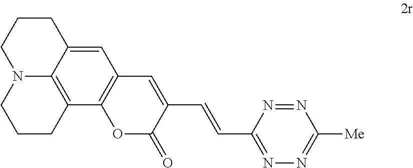 [SMILES: Cc1nnc(/C=C/c2cc3cc4c5c(c3oc2=O)CCCN5CCC4)nn1]

Cmpd 2r:

1 eq 3-iodocoumarin (Xian et. al, Mol. BioSyst., 2009, 5, 918-920), 3.0 eq base, Pd₂(dba)₃ (5%), ligand 3 (20%), microwave irradiation at 50° C. for 40 min. 4.4 mg 1a affords 6.0 mg 2r as a pink solid, in 83% yield. ¹H NMR (500 MHz, CDCl₃) δ 8.15 (d, J=16.0 Hz, 1H), 7.98 (d, J=16.0 Hz, 1H), 7.72 (s, 1H), 6.93 (s, 1H), 3.32 (dd, J=11.7, 6.0 Hz, 4H), 3.02 (s, 3H), 2.91 (t, J=6.5 Hz, 2H), 2.78 (t, J=6.3 Hz, 2H), 1.98 (dd, J=9.9, 4.5 Hz, 4H). ¹³C NMR (126 MHz, CDCl₃) δ 165.84, 165.52, 160.86, 151.71, 147.41, 144.55, 136.52, 126.09, 121.09, 119.29, 114.33, 108.82, 106.25, 50.33, 49.94, 29.84, 21.41, 21.27, 20.46, 20.31; HRMS [M+H]⁺ m/z calcd. for [C₂₀H₂₀N₅O₂]⁺ 362.1617, found 362.1611.

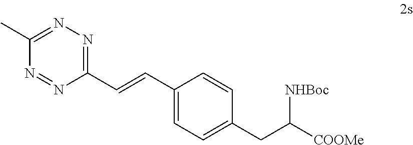 [SMILES: COC(=O)C(Cc1ccc(/C=C/c2nnc(C)nn2)cc1)NC(=O)OC(C)(C)C]

Cmpd 2s:

1.1 eq 4-bromo-N-boc-DL-phenylalanine methyl ester, 3.0 eq base, Pd₂(dba)₃ (5%), ligand 3 (20%), microwave irradiation at 50° C. for 60 min. 4.4 mg 1a affords 6.3 mg 2s as a pink solid, in 76% yield. ¹H NMR (500 MHz, CDCl₃) δ 1.41 (s, 9H), 3.05 (s, 3H), 3.07-3.17 (m, 2H), 3.73 (s, 3H), 4.62 (t, J=10 Hz, 1H), 5.04 (d, J=10 Hz, 1H), 7.21 (d, J=5 Hz, 2H), 7.44 (d, J=20 Hz, 1H), 7.61 (d, J=5 Hz, 2H), 8.27 (d, J=20 Hz, 1H). ¹³C NMR (125 MHz, CDCl₃) δ 21.4, 28.5, 38.6, 52.6, 54.5, 80.3, 120.6, 128.5, 130.3, 134.1, 138.8, 140.8, 155.8, 165.0, 166.5, 172.4; HRMS [M+H]⁺ m/z calcd. for [C₂₀H₂₅N₅O₄Na]⁺ 422.1799, found 422.1801.

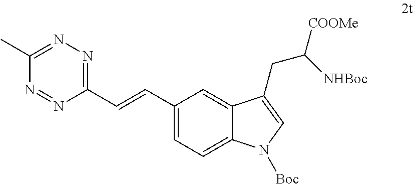 [SMILES: COC(=O)C(Cc1cn(C(=O)OC(C)(C)C)c2ccc(/C=C/c3nnc(C)nn3)cc12)NC(=O)OC(C)(C)C]

Cmpd 2t:

1.1 eq 5-bromo-N₁,N₂-boc-DL-tryptophan methyl ester, 3.0 eq base, Pd₂(dba)₃ (5%), ligand 3 (20%), microwave irradiation at 60° C. for 30 min. 4.4 mg 1a affords 5.9 mg 2t as a pink solid, in 55% yield. ¹H NMR (500 MHz, CDCl₃) δ 1.42 (s, 9H), 1.67 (s, 9H), 3.04 (s, 3H), 3.21-3.30 (m, 2H), 3.71 (s, 3H), 4.68 (d, J=5 Hz, 1H), 5.18 (d, J=10 Hz, 1H), 7.43 (m, 3H), 7.67 (d, J=10 Hz, 1H), 7.78 (s, 1H), 8.16 (s, 1H), 8.41 (d, J=15 Hz, 1H). ¹³C NMR (125 MHz, CDCl₃) δ 21.4, 28.1, 28.4, 28.5, 52.7, 53.9, 80.4, 84.5, 115.7, 116.1, 119.4, 119.7, 124.4, 125.5, 130.0, 131.3, 136.7, 141.7, 149.5, 155.3, 165.1, 166.3, 172.4; HRMS [M+Na]⁺ m/z calcd. for [C₂₇H₃₄N₆O₆Na]⁺ 561.2432, found 561.2433.

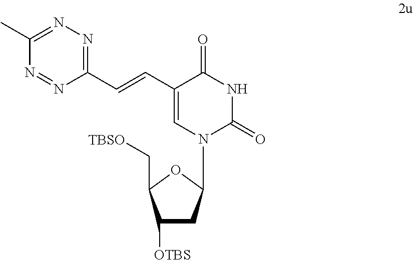 [SMILES: Cc1nnc(/C=C/c2cn([C@H]3C[C@@H](O[Si](C)(C)C(C)(C)C)[C@@H](CO[Si](C)(C)C(C)(C)C)O3)c(=O)[nH]c2=O)nn1]

Cmpd 2u:

1.1 eq 2′-deoxy-3′5′-bis-o-TBS-5-iodo-uridine (Richert et. al, Chem. Comm., 2011, 47(38), 10824-10826), 3.0 eq base, Pd₂(dba)₃ (5%), ligand 3 (20%), microwave irradiation at 50° C. for 60 min. 4.4 mg 1a affords 5.3 mg 2q as a pink solid, in 47% yield. ¹H NMR (500 MHz, CDCl₃) δ 0.09 (m, 12H), 0.90 (s, 9H), 0.92 (s, 9H), 2.04-2.07 (m, 1H), 2.39-2.41 (m, 1H), 3.02 (s, 3H), 3.79-3.81 (m, 1H), 3.92-3.94 (m, 1H), 4.23 (m, 1H), 4.42 (m, 1H), 6.33 (dd, J=10, 5 Hz, 1H), 7.89 (dd, J=20, 5 Hz, 1H), 8.09 (m, 1H), 8.75 (s, 1H). ¹³C NMR (125 MHz, CDCl₃) δ −5.5, −5.4, −5.0, −4.7, 17.9, 18.4, 21.1, 25.59, 25.62, 25.74, 25.84, 42.07, 63.0, 72.4, 86.2, 88.5, 110.4, 122.1, 132.5, 141.9, 148.8, 161.0, 164.8, 166.1; HRMS [M+Na]-m/z calcd. for [C₂₆H₄₄N₆O₅Si₂Na]⁺ 599.2804, found 599.2805.

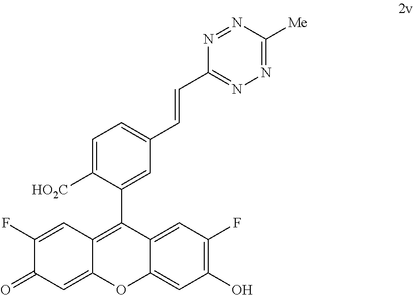 [SMILES: Cc1nnc(/C=C/c2ccc(C(=O)O)c(-c3c4cc(F)c(=O)cc-4oc4cc(O)c(F)cc34)c2)nn1]

Cmpd 2v:

1.1 eq 4-bromo-Oregon Green 488, 4 eq base, Pd₂(dba)₃ (10%), ligand 3 (40%), microwave at 50° C. for 40 min. 4.4 mg 1a afford 5.6 mg 2v as a dark orange solid, in 58% yield. ¹H NMR (500 MHz, DMSO-D6) δ 10.79 (s, 1H), 8.48 (s, 1H), 8.43 (d, J=16.3 Hz, 1H), 8.33 (dd, J=8.2, 1.4 Hz, 1H), 7.88 (d, J=16.4 Hz, 1H), 7.38 (d, J=8.0 Hz, 1H), 6.89 (d, J=7.5 Hz, 2H), 6.61 (d, J=11.2 Hz, 2H), 2.97 (s, 3H). ¹³C NMR (126 MHz, DMSO-D6) δ 167.96, 166.53, 164.16, 152.66, 147.79, 147.39, 147.02, 138.08, 137.60, 137.43, 136.40, 135.09, 124.95, 124.49, 123.55, 118.18, 116.94, 114.15, 113.98, 105.14, 104.85, 20.98; HRMS [M+H]-m/z calcd. for [C₂₅H₁₄F₂N₄O₅]⁺ 489.1005, found 489.1006.

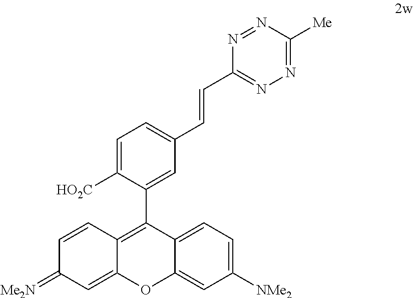

Cmpd 2w:

1.1 eq 9-(5-bromo-2-carboxyphenyl)-3,6-bis(dimethylamino) rhodamine (Peng et. al, Org. Lett., 2013, 15, 492-495), 4 eq base, Pd₂(dba)₃ (10%), ligand 3 (40%), microwave at 50° C. for 40 min. 4.4 mg 1a afford 5.4 mg 2w as a dark pink solid, in 53% yield. ¹H NMR (500 MHz, CDCl₃) δ 8.25 (d, J=16.3 Hz, 1H), 8.05 (d, J=8.0 Hz, 1H), 7.85 (dd, J=8.0, 1.3 Hz, 1H), 7.51-7.44 (m, 2H), 6.67 (s, 1H), 6.65 (s, 1H), 6.51 (d, J=2.6 Hz, 2H), 6.42 (d, J=2.6 Hz, 1H), 6.40 (d, J=2.6 Hz, 1H), 3.08 (s, 3H), 2.99 (s, 12H). ¹³C NMR (126 MHz, CDCl₃) δ 175.11, 169.32, 163.91, 154.43, 153.04, 152.24, 141.57, 138.95, 129.40, 128.90, 128.61, 125.58, 124.33, 123.11, 108.83, 106.46, 98.69, 40.40, 21.48; HRMS [M+H]⁺ m/z calcd. for [C₂₉H₂₈N₆O₃]⁺ 508.2223, found 508.2178.

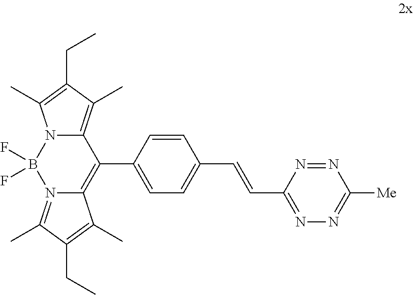

Cmpd 2x:

1 eq 2,6-diethyl-4,4-difluoro-1,3,5,7-tetramethyl-8-(4-iodophenyl)-4-bora-3a,4a-diaza-sinadcene (Akkaya et. al, J. Am. Chem. Soc., 2006, 128, 14474-14475), 3 eq base, Pd₂(dba)₃ (5%), ligand 3 (20%), microwave at 50° C. for 35 min. 4.4 mg 1a afford 8.2 mg 2× as a dark orange solid, in 83% yield. ¹H NMR (500 MHz, CDCl₃) δ 8.38 (d, J=16.3 Hz, 1H), 7.82 (d, J=8.1 Hz, 2H), 7.56 (d, J=16.3 Hz, 1H), 7.39 (d, J=8.2 Hz, 2H), 3.09 (s, 3H), 2.55 (s, 6H), 2.31 (q, J=7.5 Hz, 4H), 1.34 (s, 7H), 0.99 (t, J=7.6 Hz, 6H). ¹³C NMR (126 MHz, CDCl₃) δ 167.03, 165.13, 154.60, 140.48, 139.56, 138.67, 138.30, 136.03, 133.50, 131.02, 129.71, 129.12, 122.05, 21.76, 17.58, 15.13, 13.06, 12.41; HRMS [M+Na]+m/z calcd. for [C₂₈H₃₁BF₂N₆Na]⁺ 523.2569, found 523.2569.

### Example 15

### Synthesis of dialkyl-s-tetrazine 11 by hydrogenation of (E)-alkenyl-1,2,4,5-tetrazine 2a

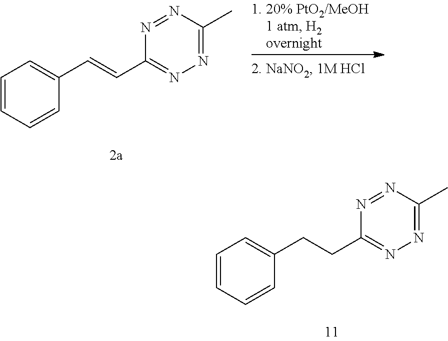

PtO₂ (0.46 mg, 0.002 mmol) was added to a stirring solution of compound 2a (4.0 mg, 0.02 mmol) in methanol (2.0 mL) at room temperature. The reaction solution was hydrogenated under 1 atm hydrogen gas. After 3 hours, TLC indicated that half of the starting material remained and the reaction was proceeding slowly. Another 10% PtO₂ was added and stirring was continued overnight, at which point TLC indicated that the reaction had completed. The reaction was worked up by the addition of NaNO₂/1M HCl. EtOAc (20 mL) was added to the solution and washed with brine (20 mL). The organic layer was dried over Na₂SO₄ and evaporated. The residue was isolated by a TLC plate (Hexanes:Et₂O=3:1) to afford 3.2 mg 11 as a pink solid, in 81.6% yield. ¹H NMR (500 MHz, CDCl₃) δ 3.03 (s, 3H), 3.28 (t, J=7.5 Hz, 2H), 3.62 (t, J=7.5 Hz, 2H), 7.19-7.30 (m, 5H). ¹³C NMR (125 MHz, CDCl₃) δ 21.2, 34.1, 36.4, 126.6, 128.5, 128.7, 140.0, 167.6, 169.3; HRMS [M+H]⁺ m/z calcd. For [C₁₁H₁₃N₄]⁺ 201.1135, found 201.1128.

### Example 16

### Synthesis of the Unnatural Deoxyribose 12

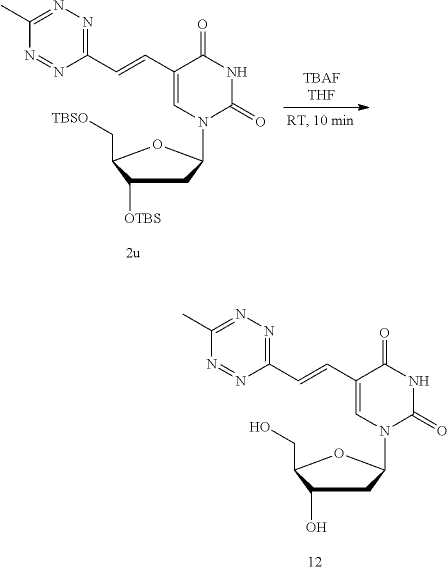 [SMILES: Cc1nnc(/C=C/c2cn([C@H]3C[C@@H](O[Si](C)(C)C(C)(C)C)[C@@H](CO[Si](C)(C)C(C)(C)C)O3)c(=O)[nH]c2=O)nn1 ; Cc1nnc(/C=C/c2cn([C@H]3C[C@@H](O)[C@@H](CO)O3)c(=O)[nH]c2=O)nn1]

TBAF (14 μL, 1.0 M in THF) was added to a stirred solution of compound 2u (8.0 mg, 0.014 mmol) in dry THF (2.0 mL) at room temperature. The reaction solution was stirred at room temperature for 10 min, at which point TLC indicated that the starting material was consumed. The solvent was evaporated and the residue was purified by a TLC plate (EtOAc) to afford 3.7 mg of compound 12 as a pink solid, in 76% yield. ¹H NMR (500 MHz, CDCl₃) δ 2.25-2.35 (m, 1H), 2.93 (s, 3H), 3.76 (dd, J=15, 5 Hz, 1H), 3.86 (dd, J=15, 5 Hz, 1H), 3.94 (dd, J=10, 5 Hz, 1H), 4.06 (m, 1H), 6.27 (t, J=5 Hz, 1H), 7.94 (dd, J=25, 15 Hz, 1H), 8.58 (s, 1H). ¹³C NMR (125 MHz, CDCl₃) δ 19.5, 40.6, 61.1, 70.4, 87.8, 87.9, 110.0, 120.4, 133.1, 143.1, 149.8, 162.5, 165.0, 166.2; HRMS [M+Na]⁺ m/z calcd. for [C₁₄H1₆N₆O₅Na]⁺ 371.1074, found 371.1075.

### Example 17

### Synthesis of Unnatural Amino Acids 13 and 14

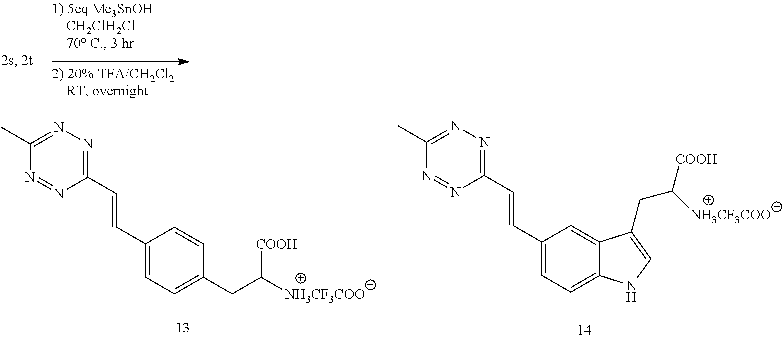

In a 50 mL flask, 2s or 2t (1.0 eq) was dissolved in 1,2-dichloroethane, followed by Me₃SnOH (5.0 eq). The solution was heated to 70° C. and stirred for 3 hours. The reaction solution was evaporated and purified by a TLC plate (CH₂Cl₂: MeOH=20:1) to afford the carboxylic acid intermediate. The intermediate was dissolved in 20% CF₃COOH in CH₂Cl₂ and stirred at room temperature overnight. The reaction solution was evaporated and purified by a TLC plate (CH₂Cl₂:MeOH:CF₃COOH=5:1:0.1, the silica was washed with CH₂Cl₂:MeOH:CF₃COOH=3:1:0.1) to afford the trifluoroacetic acid salt of the unnatural amino acid as a solid.

Cmpd 13:

5.0 mg 2s afforded 3.0 mg 6 as pink solid, in 85% yield. ¹H NMR (500 MHz, CDCl₃) δ 2.98 (s, 3H), 3.21 (m, 2H), 4.29 (bs, 1H), 7.40 (d, J=5 Hz, 2H), 7.53 (d, J=15 Hz, 1H), 7.76 (d, J=5 Hz, 2H), 8.27 (d, J=15 Hz, 1H). ¹³C NMR (125 MHz, CDCl₃) δ 19.6, 35.9, 120.8, 128.4, 129.9, 134.8, 136.7, 139.6, 164.7, 166.4; HRMS [M+H]⁺ m/z calcd. for [C₁₄H₁₆N₅O₂]⁺ 286.1299, found 286.1298.

Cmpd 14:

6.5 mg 2t afforded 5.1 mg 7 as orange solid, in 95% yield. ¹H NMR (500 MHz, CD₃OD) δ 2.95 (s, 3H), 3.38-3.42 (dd, J=15, 10 Hz, 1H), 3.50 (m, 1H), 4.31 (bs, 1H), 7.26 (s, 1H), 7.43 (m, 2H), 7.62 (d, J=5 Hz, 1H), 7.96 (s, 1H), 8.39 (d, J=15 Hz, 1H), 10.93 (bs, 1H). ¹³C NMR (125 MHz, CD₃OD) δ 21.9, 27.4, 109.1, 113.4, 118.2, 121.1, 122.4, 127.0, 128.2, 128.7, 139.5, 143.8, 166.4, 167.2; HRMS [M−H]f m/z calcd. for [C₁₆H₁₅N₆O₂]⁻ 323.1262, found 323.1259.

### Example 18

### Stability Studies of a Series of Tetrazines 2a, 2b, and 2c

Cmpds 2a-2c at 1 mM were incubated in 50% DMF, 50% phosphate-buffered saline (PBS) 1× standard buffer pH 7.4 (Sigma Life Science) at 22° C. room temperature. A NANODROP™ 2000c (Thermo Scientific) UV-Vis absorption scan was used to do the measurements of samples in quartz cuvettes. Disappearance of the characteristic tetrazine absorption peak intensity at around 520 nm was measured over time and baseline-adjusted. Absorbance peak maxima varied slightly between the samples, and the peak intensity values were averaged at 524-527 nm for 2a, 535-539 nm for 2b, and 519-525 nm for 2c. Baselines were estimated by extrapolating a straight line by using the absorbance intensities immediately preceding and following the tetrazine peak.

### Example 19

### Tetrazine Kinetic Measurements

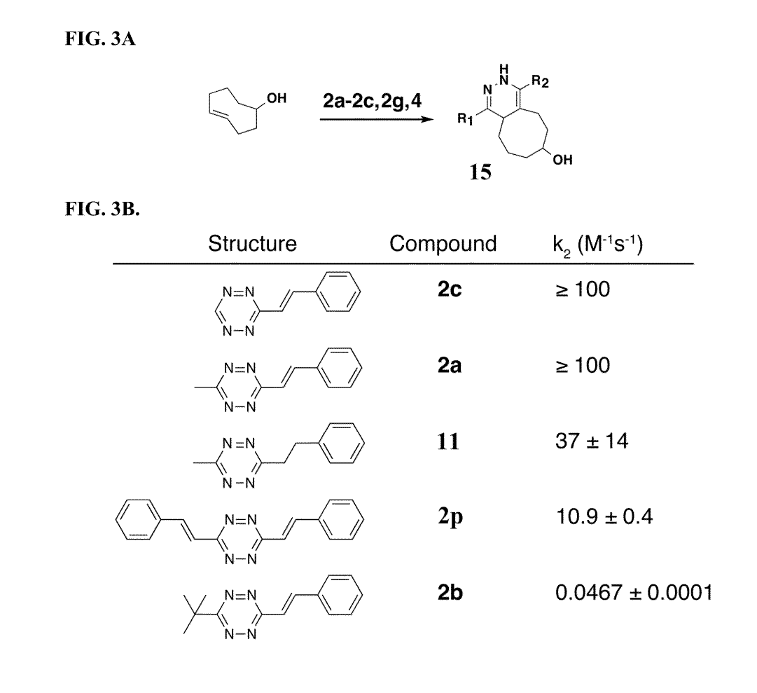

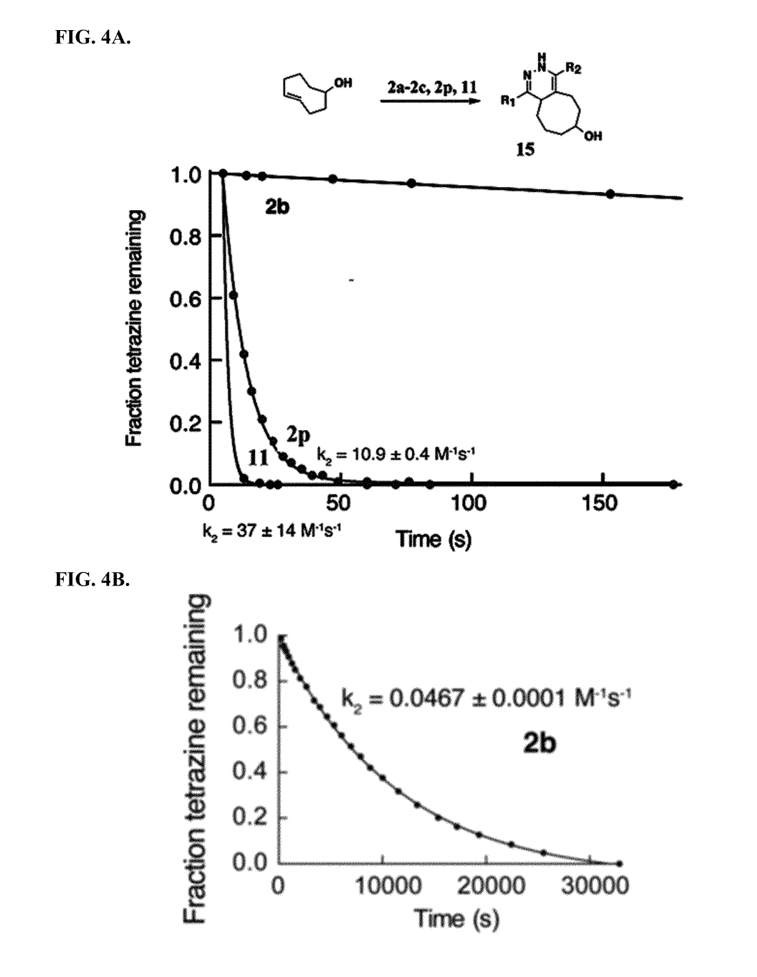

A NANODROP™ 2000c (Thermo Scientific) equipped with a cuvette reader and a stirrer was used in the kinetic measurements, and the disappearance of the tetrazine peak absorption around 520 nm was measured over time. Tetrazines at 1 mM were reacted with an excess 10 mM (E)-cyclooct-4-enol with the measurements commencing immediately upon (E)-cyclooct-4-enol addition (FIGS. 3A, 4A-4B). Solution conditions were maintained at 1:1 DMF:PBS pH 7.4 buffer (Sigma Life Science), and all the measurements were done at 22° C. room temperature. Tetrazine peak intensity at each time point was adjusted for background by extrapolation, as in the previous section. Tetrazine peak disappearance is shown as points in FIGS. 4A-4B, with the connecting lines resulting from the nonlinear fits made with Prism 6.0c. Least squares exponential decay fits were done by using the equation:

y=(y₀−Plateau)×e^{Kx}+Plateau

In order to determine the pseudo first-order reaction rate constants. Conversion to the second order rate constants was done by employing the equation:

$${k}_{2} = \frac{{k}_{obs}}{{[ TCO ]}_{0}} .$$

### Example 20

### Characterization of the Reaction Between Tetrazine 2a and (E)-Cyclooct-4-enol (TCO)

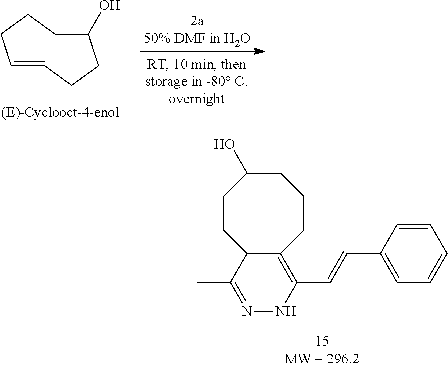 [SMILES: OC1CC/C=C/CCC1 ; CC1=NNC(/C=C/c2ccccc2)=C2CCCC(O)CCC12]

Tetrazine 2a (250 mM in DMF, 5 μL) and (E)-Cyclooct-4-enol (TCO) (250 mM in DMF, 6 μL) were combined in 125 μL of H₂O and 114 μL DMF at a final concentration of 5 mM for tetrazine 2a and 6 mM for TCO. The reaction solution was agitated for 10 minutes at room temperature and stored at −80° C. overnight and then analyzed by LC-MS.

### Example 21

### Characterization of the Reaction Product 5 Between Tetrazine 2v and Cyclopropene 4

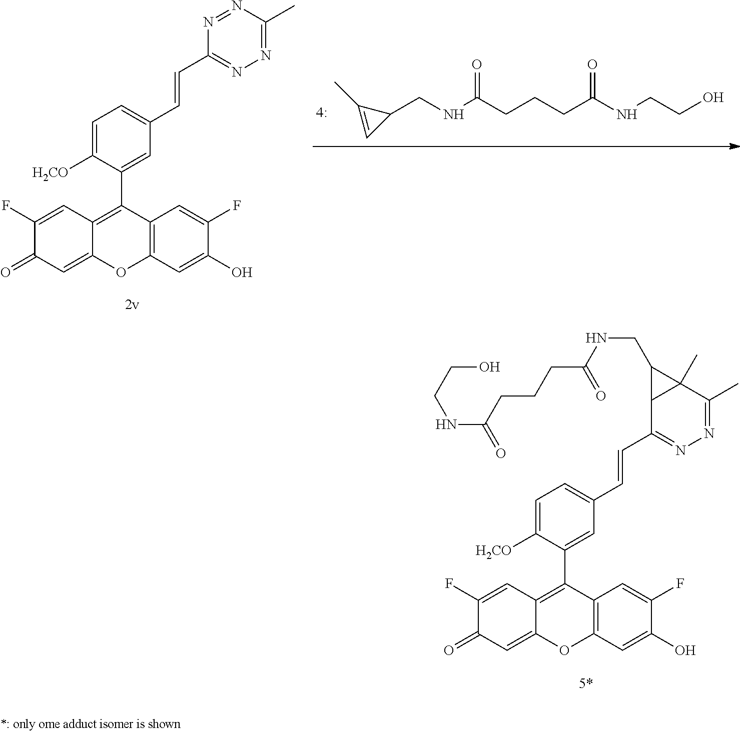

Tetrazine 2v (250 mM in DMF, 5 μL) and cyclopropene 4 (250 mM in DMF, 6 μL) were combined in 50 μL of H₂O and 189 μL DMF at a final concentration of 5 mM for tetrazine 2v and 6 mM for 4. The reaction solution was placed on a shaker for 30 minutes at room temperature then analyzed by LC-MS and HRMS.

### Example 22

### Characterization of the Reaction Between Tetrazine 2v and (E)-Cyclooct-4-Enol (TCO) Product 6

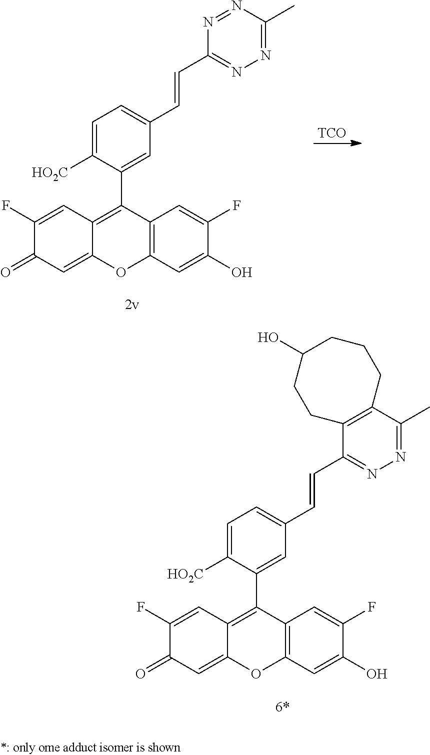 [SMILES: Cc1nnc(/C=C/c2ccc(C(=O)O)c(-c3c4cc(F)c(=O)cc-4oc4cc(O)c(F)cc34)c2)nn1 ; "TCO" ; Cc1nnc(/C=C/c2ccc(C(=O)O)c(-c3c4cc(F)c(=O)cc-4oc4cc(O)c(F)cc34)c2)c2c1CCCC(O)CC2]

Tetrazine 2v (250 mM in DMF, 5 μL) and TCO (250 mM in DMF, 6 μL) were combined in 50 μL of H₂O and 189 μL DMF at a final concentration of 5 mM for tetrazine 2v and 6 mM for TCO. The reaction solution was placed on a shaker for 30 minutes then analyzed by LC-MS and HRMS.

### Example 23

### Characterization of the Reaction Product 7 Between Tetrazine 2w and Cyclopropene 4

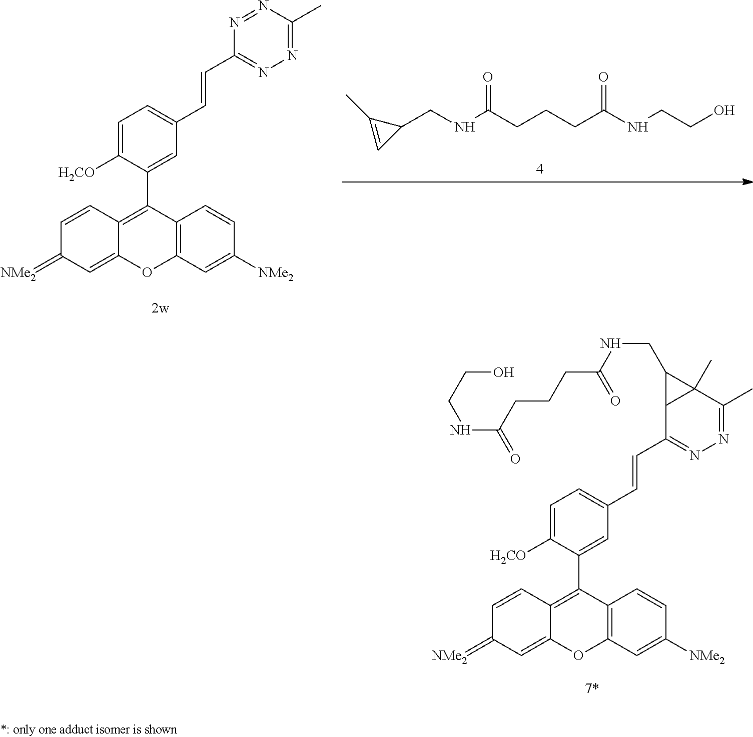

Tetrazine 2w (250 mM in DMF, 5 μL) and cyclopropene 4 (250 mM in DMF, 6 μL) were combined in 50 μL of H₂O and 189 μL DMF at a final concentration of 5 mM for tetrazine 2w and 6 mM for 4. The reaction solution was on shaker for 30 minutes at room temperature then analyzed by LC-MS and HRMS.

### Example 24

### Characterization of the Reaction Between Tetrazine 2w and (E)-Cyclooct-4-Enol (TCO) and DDQ Oxidation Product 8

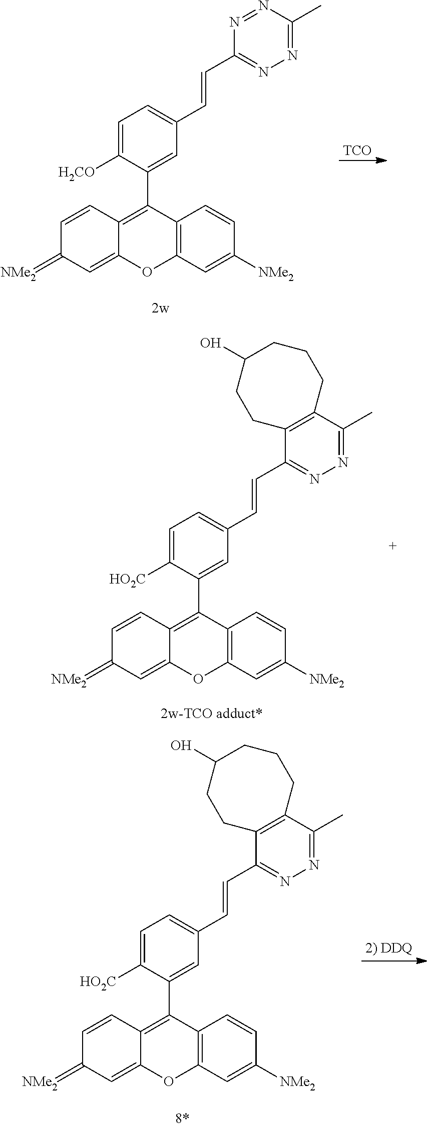

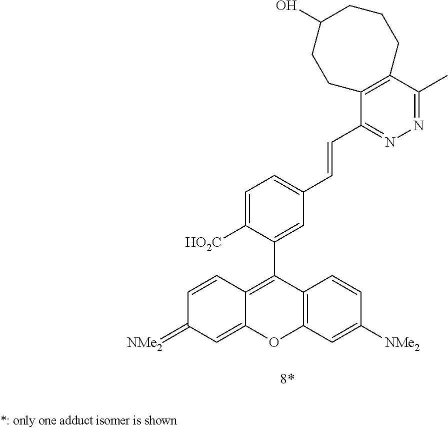

Tetrazine 2w (250 mM in DMF, 5 μL) and (E)-Cyclooct-4-enol (TCO) (250 mM in DMF, 6 μL) were combined in 239 μL DMF at a final concentration of 5 mM for tetrazine 2w and 6 mM for TCO. The reaction solution was on shaker for 10 minutes at room temperature then 1.25 μmol 2,3-Dichloro-5,6-dicyano-1,4-benzoquinone (DDQ) was added, after 1 minute 10 μL of water was added to quench the reaction. Both two steps were analyzed by LC-MS and HRMS.

### Example 25

### Characterization of the Reaction Product 9 Between Tetrazine 2x and Cyclopropene 4

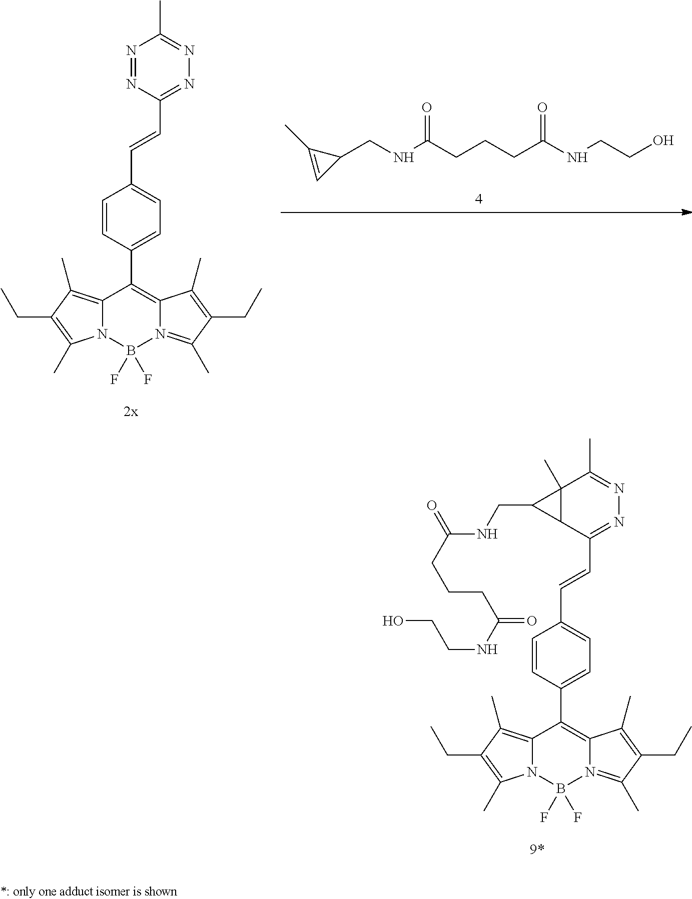

Tetrazine 2x (250 mM in DMF, 5 μL) and cyclopropene 4 (250 mM in DMF, 6 μL) were combined in 50 μL of H₂O and 189 μL DMF at a final concentration of 5 mM for tetrazine 2x and 6 mM for 4. The reaction solution was placed on a shaker for 30 minutes at room temperature then analyzed by LC-MS and HRMS.

### Example 26

### Characterization of the Reaction Between Tetrazine 2x and (E)-Cyclooct-4-Enol (TCO) and DDQ Oxidation Product 10

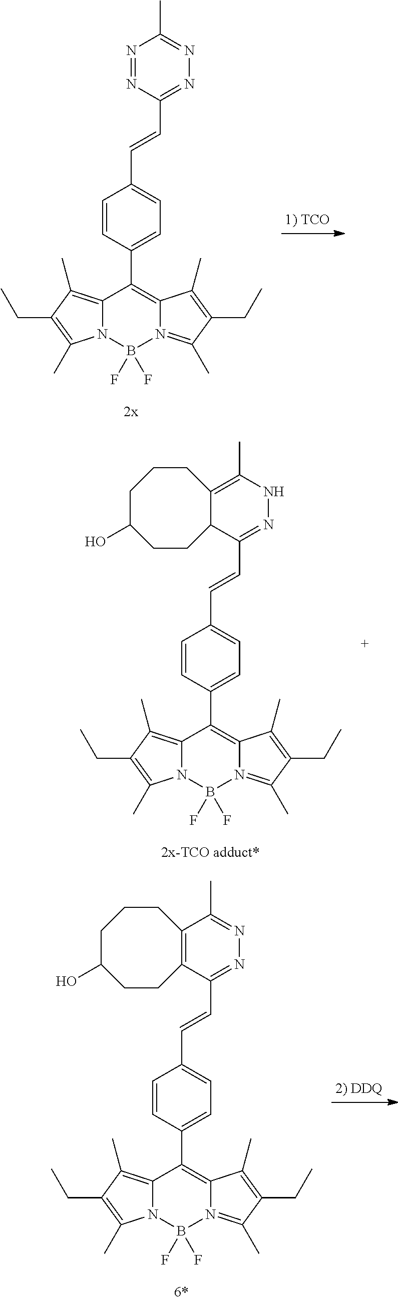

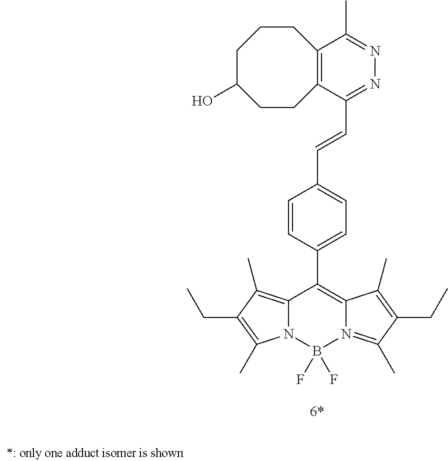

Tetrazine 2x (250 mM in DMF, 5 L) and (E)-Cyclooct-4-enol (TCO) (250 mM in DMF, 6 μL) were combined in 239 L DMF at a final concentration of 5 mM for tetrazine 2x and 6 mM for TCO. The reaction solution was on shaker for 10 minutes at room temperature then 1.25 μmol 2,3-Dichloro-5,6-dicyano-1,4-benzoquinone (DDQ) was added, after 1 minute, 10 μL of water was added to quench the reaction. Both steps were analyzed by LC-MS and HRMS.

### Example 27

### Fluorescence Emission Spectra of Turn-on Reaction

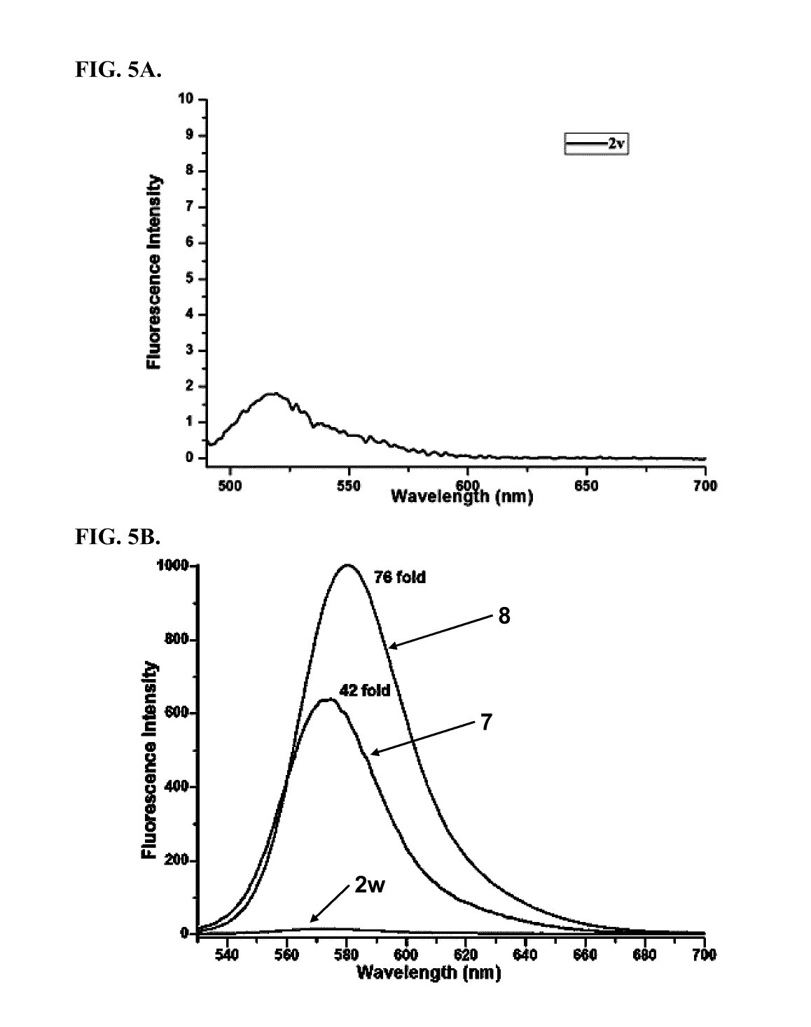

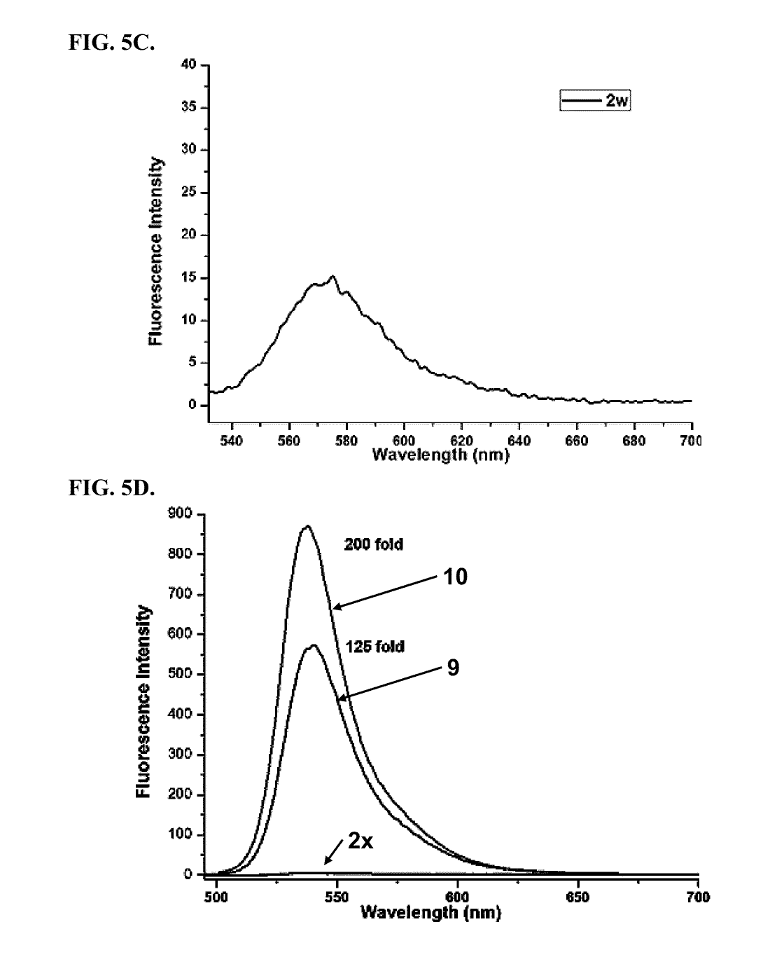

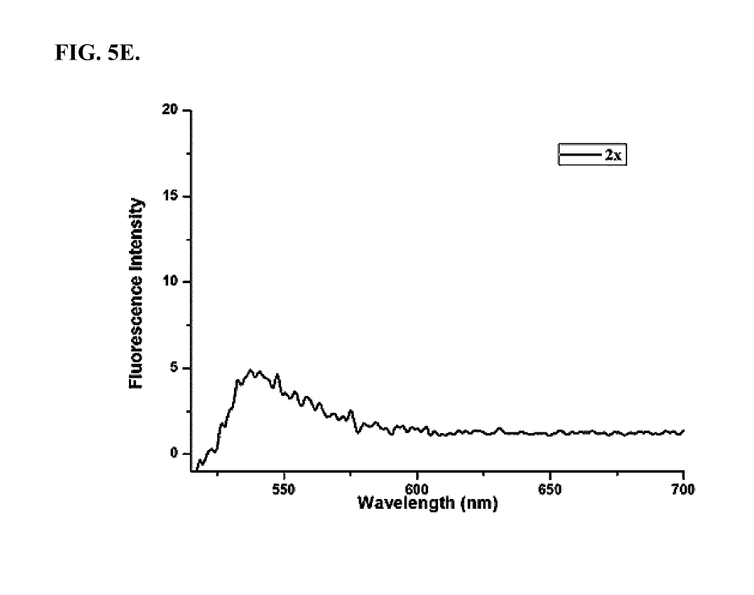

Fluorescence emission of 2v, 5, 6 are depicted in FIG. 1A. All three compounds were dissolved in 2 μM phosphate-buffered saline (PBS) at pH 7.4. FIG. 5A depicts fluorescence emission of 2v in 2 μM phosphate-buffered saline (PBS) at pH 7.4. FIG. 5B depicts fluorescence emission of 2w, 7, 8. All three compounds were dissolved in 2 μM EtOH solution. Compound 2w and 8 were tested after HPLC purification and 7 was tested directly after reaction. FIG. 5C depicts fluorescence emission of 2w in 2 μM EtOH solution. FIG. 5D depicts fluorescence emission of 2x, 9, 10. All three compounds were dissolved in 2 μM EtOH solution. Compounds 2x and 10 were tested after HPLC purification and compound 9 was tested directly after reaction. FIG. 5E depicts fluorescence emission of 2x in 2 μM EtOH solution.

### Embodiments

An embodiment of the disclosed method is set forth in embodiment P1 following.

### Embodiment P1

A method for synthesizing a 3-substituted-6-alkenyl-1,2,4,5-tetrazine having the formula:

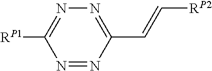

wherein: R^{P1} is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl; R^{P2} is substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl; X is halogen; and PPG is a protecting group. The method includes (i) contacting a first compound with structure of formula

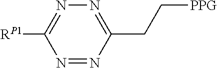

with a second compound of formula R^{P2}—X in a solvent system, said solvent system comprising 1,2,3,4,5-Pentaphenyl-1′-(di-tert-butylphosphino)ferrocene, Pd₂(dba)₃, and a base; and (ii) irradiating said solvent system with microwave radiation; thereby synthesizing said 3-substituted-6-alkenyl-1,2,4,5-tetrazine.

### IV. References

[1]a) G. Clavier, et al., Chem. Rev. 2010, 110, 3299-3314; b) N. K. Devaraj & R. Weissleder, Acc. Chem. Res. 2011, 44, 816-827; c) G. Budin, et al., Angew. Chem. Int. Ed. 2011, 50, 9378-9381.
[2]a) D. E. Chavez, et al., Angew. Chem. Int. Ed. 2013, 52, 6876-6879; b) J. Yang, et al., Angew. Chem. Int. Ed. 2012, 51, 7476-7479; c) A. Hamasaki, et al., J. Am. Chem. Soc. 2005, 127, 10767-10770; d) W. Kaim, Coordin. Chem. Rev. 2002, 230, 127-139; e) Z. Li, et al., J. Am. Chem. Soc. 2010, 132, 13160-13161; f) N. Saracoglu, Tetrahedron 2007, 63, 4199-4236.
[3]a) K. S. Yang, et a 1., Angew. Chem. Int. Ed. 2012, 51, 6598-6603; b) T. Plass, et al., Angew. Chem. Int. Ed. 2012, 51, 4166-4170; c) K. Lang, et al., Nat. Chem. 2012, 4, 298-304.
[4]N. K. Devaraj, et al., Angew. Chem. Int. Ed. 2010, 49, 2869-2872.
[5]J. C. T. Carlson, et al., Angew. Chem. Int. Ed. 2013, 52, 6917-6920.
[6]a) I. D. L. Albert, et al., J. Am. Chem. Soc. 1997, 119, 6575-6582; b) P. Audebert, et al., Electrochem. Comm. 2004, 6, 144-147; c) P. Audebert, et al., New J. Chem. 2004, 28, 387-392; d) W. B. Ma, et al., J. Mol. Struc-Theochem. 2006, 774, 13-18.
[7]J. Yang, et al., Angew. Chem. Int. Ed. 2012, 51, 5222-5225.
[8]G. D. Byrkit & G. A. Michalek, Ind. Eng. Chem. 1950, 42, 1862-1875.
[9]a) N. Leconte, et al., Synlett 2007, 204-210; b) Z. Novak & A. Kotschy, Org. Lett. 2003, 5, 3495-3497.
[10]A. C. Knall & C. Slugovc, Chem. Soc. Rev. 2013, 42, 5131-5142.
[11]S. Pican, et al., Synlett. 2009, 731-734.
[12]a) C. F. B. Praquin, et al., Org. Proc. Res. Dev. 2011, 15, 1124-1129; b) G. K. S. Prakash, et al., J. Am. Chem. Soc. 2011, 133, 2140; c) A. S. Saiyed & A. V. Bedekar, Tetrahedron Letters 2010, 51, 6227-6231.
[13]V. P. Mehtaa & E. V. Van der Eycken, Chem. Soc. Rev. 2011, 40, 4925-4936.
[14]a) J. P. Stambuli, et al., J. Am. Chem. Soc. 2001, 123, 2677-2678; b) M. R. Netherton & G. C. Fu, Org. Lett. 2001, 3, 4295-4298; c) Q. Shelby, et al., J. Am. Chem. Soc. 2000, 122, 10718-10719; d) N. Kataoka, et al., J Org. Chem. 2002, 67, 5553-5566.
[15]T. Lifka & H. Meier, J. Prak. Chem.-Chem. Ztg. 1995, 337, 641-646.
[16]a) J. Seckute, et al., Nucleic Acids Research 2013, 41, e148; b) K. Lang, L. et al., J. Am. Chem. Soc. 2012, 134, 10317-10320; c) J. Schoch, et al., J. Am. Chem. Soc. 2010, 132, 8846-8847.
[17]M. R. Karver, et al., Bioconjugate Chem. 2011, 22, 2263-2270.
[18]C. M. Cole, et al., Chembiochem 2013, 14, 205-208.
[19]C. Dumas-Verdes, et al., Eur JOrg Chem. 2010, 2525-2535.
[20]G. Ulrich, et al., Angew. Chem. Int. Ed. 2005, 44, 3694-3698.
[21]A. Loudet & K. Burgess, Chem Rev 2007, 107, 4891-4932.
[22]a) A. Burghart, et al., Chem. Comm. 2000, 2203-2204; b) G. S. Jiao, et al., J. Am. Chem. Soc. 2003, 125, 14668-14669.
[23]J. Yang, et al., Chem. A Eur. J. 2014, 20, 3365-3375.
[24]N. K. Devaraj, et al., P. Natl. Acad. Sci. USA. 2012, 109, 4762-4767.

What is claimed is:

1. A method of synthesizing a compound of Formula I: 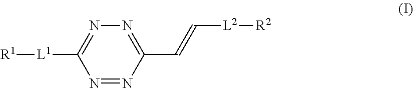
the method comprising reacting a compound of Formula II: 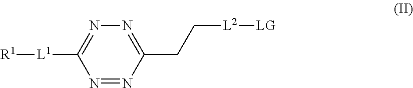
with a compound of Formula III:
R²—X (III),
wherein the compounds of Formula II and III are reacted in the presence of a Pd catalyst and a ligand under basic conditions, and further wherein:
L¹ and L² are independently a bond or a covalent linker;
X is halogen;
R¹ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a biomolecule, a dye or fluorophore;
R² is substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl or a biomolecule, a dye or fluorophore; and
LG is a leaving group.

2. The method of claim 1, wherein LG is an electron withdrawing group.

3. The method of claim 1, wherein:
LG is —N₂⁺, —OR^{3A}₂⁺, —OSO₂R^{3A}F, a perfluoroalkylsulfonate, a tosylate, a mesylate, a halogen; —OH₂⁺, —ONO₂, —OPO(OH)₂, —ONO₂, —S(R^{3B}R^{3C})⁺, —N(R^{3A}R^{3B}R^{C})⁺, —OCOR^{3A}, substituted or unsubstituted aryloxy or substituted or unsubstituted heteroaryloxy; and
R^{3A}, R^{3B} and R^{3C} are independently hydrogen, halogen, —CF₃, —CCl₃, —CBr₃, —CI₃, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)—OH, —NHOH, —OCF₃, —OCCl₃, —OCBr₃, —OCI₃, —OCHF₂, —OCHC1₂, —OCHBr₂, —OCHI₂, substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl; R^{3B} and R^{3C} substituents bonded to the same nitrogen atom may optionally be joined to form a substituted or unsubstituted heterocycloalkyl or substituted or unsubstituted heteroaryl.

4. The method of claim 1, wherein the method further comprises a solvent.

5. The method of claim 4, wherein the solvent is an organic solvent.

6. The method of claim 1, wherein the method comprises an organic base.

7. The method of claim 1, wherein the method is conducted at a temperature of from about 25° C. to 200° C.

8. The method of claim 1, wherein the method is conducted using electromagnetic radiation.

9. The method of claim 1, wherein the method is microwave-assisted.

10. The method of claim 9, wherein the method is conducted at a temperature of from about 40° C. to 80° C.

11. The method of claim 1, wherein the method further comprises a co-catalyst.

12. The method of claim 1, wherein the method is a cascade reaction or one-pot procedure.

13. The method of claim 1, wherein the ligand is a phosphinoferrocene ligand.

14. The method of claim 13, wherein the ligand is 1,2,3,4,5-pentaphenyl-1′-(di-tert-butylphosphino)ferrocene.

15. The method of claim 1, wherein the catalyst is Pd₂(dba)₃.

16. The method of claim 6, wherein the base is trimethylamine or dicyclohexylmethylamine.

17. The method of claim 1, wherein the compound of Formula I is a 3-substituted-6-alkenyl-1,2,4,5-tetrazine.

18. The method of claim 1, wherein X is iodine.

19. The method of claim 1, wherein LG is −OMs.

20. The method of claim 1, wherein L¹ and L² are independently substituted or unsubstituted C₁-C₆ alkylene.

21. The method of claim 1, further comprising synthesizing a compound of Formula IV: 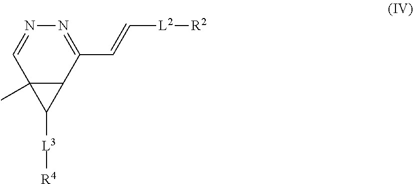
by reacting the compound of Formula I with a compound of Formula V: 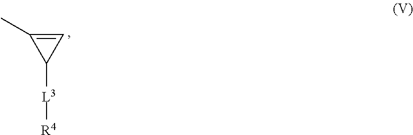
wherein:
L³ is a bond or covalent linker; and
R⁴ is a biomolecule, a dye or fluorophore.

22. The method of claim 21, wherein L³ is substituted or unsubstituted C₁-C₆ alkylene.

23. The method of claim 1, wherein the dye is a xanthene dye or a boron-dipyrromethene dye.

24. A compound of Formula I: 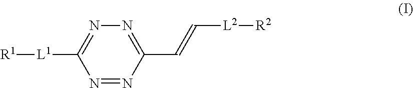
wherein:
L¹ and L² are independently a bond or linker;
R¹ is hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, a biomolecule, a dye or fluorophore; and
R² is substituted or unsubstituted alkyl, substituted or unsubstituted heteroalkyl, substituted or unsubstituted cycloalkyl, substituted or unsubstituted heterocycloalkyl, substituted or unsubstituted aryl, or substituted or unsubstituted heteroaryl or a biomolecule, a dye or fluorophore.

25. The compound of claim 24, wherein L¹ and L² are independently a bond.

26. The compound of claim 25, wherein R¹ is substituted or unsubstituted alkyl, substituted or unsubstituted aryl or substituted or unsubstituted heteroaryl.

27. The compound of claim 25, wherein R² is substituted or unsubstituted alkyl, substituted or unsubstituted heterocycloalkyl, substituted or unsubstituted aryl or substituted or unsubstituted heteroaryl.

28. The compound of claim 25, wherein the compound is:

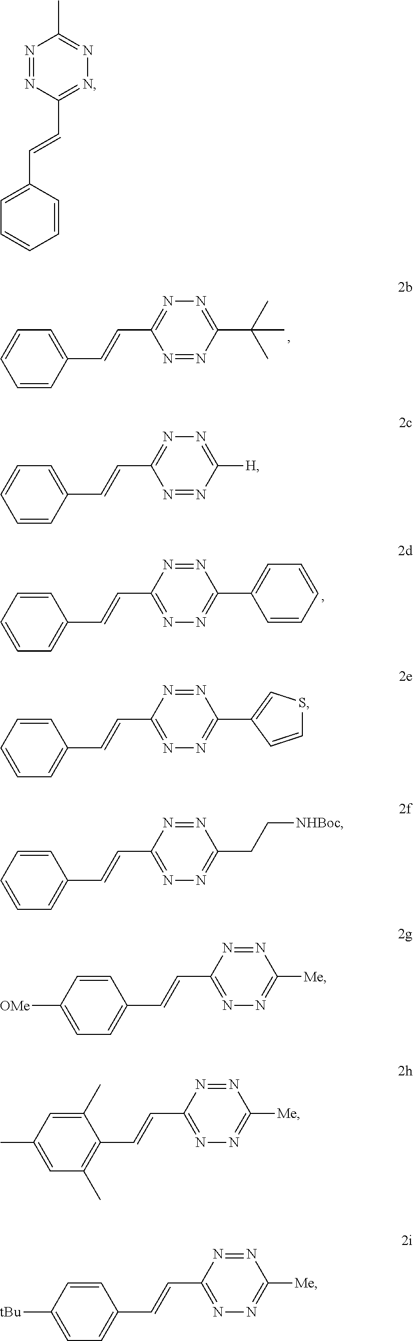

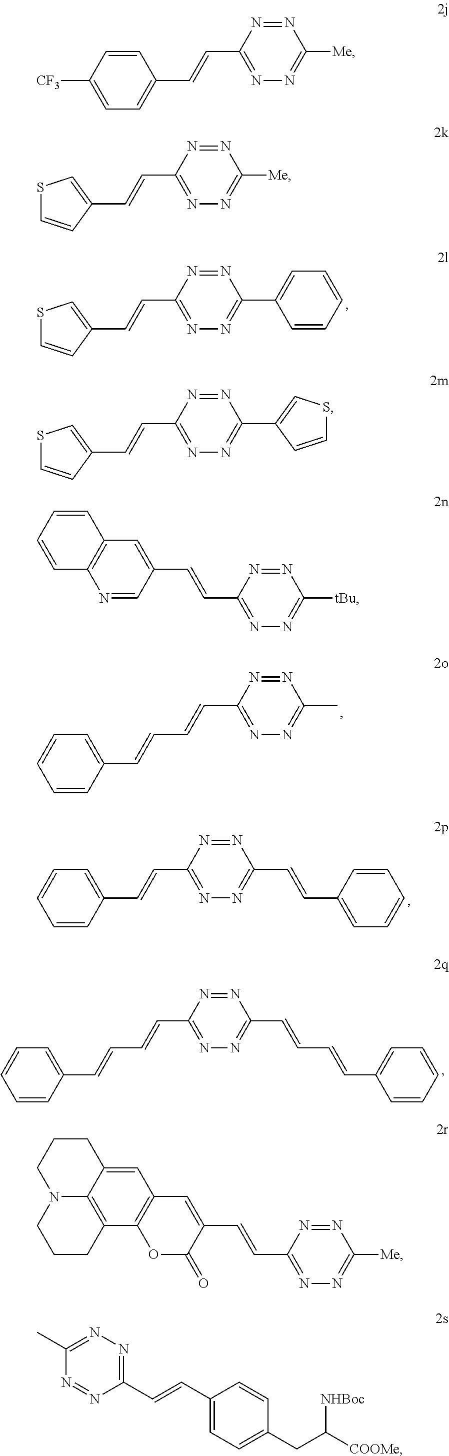

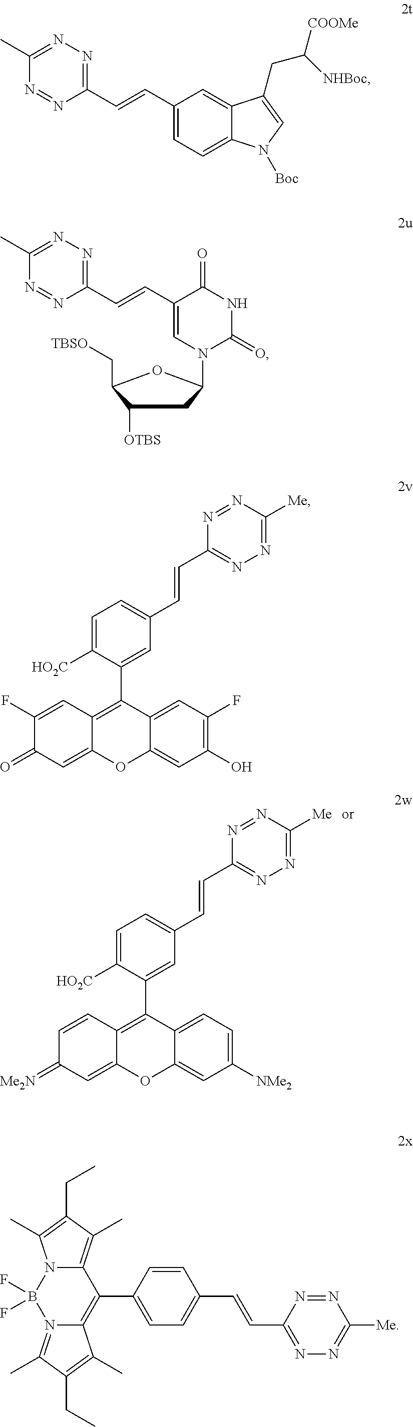

29. A compound of Formula IV: 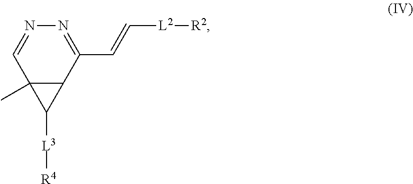
wherein:
L² and L³ are independently a bond or linker;
R² is substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl or a biomolecule, a dye or fluorophore; and
R⁴ is a biomolecule, a dye or fluorophore.

30. A method of detecting a biomolecule of interest, comprising:
(i) contacting the compound of Formula I of claim 23, wherein R² is a fluorophore, with a compound of Formula V: 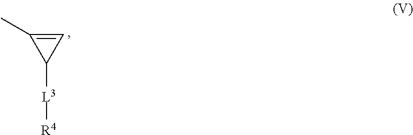
wherein:
L³ is a covalent bond or linker; and R⁴ is the biomolecule; and
(ii) detecting the level of fluorescence, wherein an increase in fluorescence compared to a control is indicative of the presence of the biomolecule.

31. The method of claim 30, wherein the biomolecule is in a cell.

32. The method of claim 31, wherein the cell is a live cell.

33. The method of claim 31, wherein the cell is a human cell.

34. The method of claim 30, wherein the biomolecule is a lipid, nucleic acid, protein or carbohydrate.

35. The method of claim 30, wherein the biomolecule is a phospholipid or antibody.
